(19) United States
(12) Patent Application Publication
(10) Pub. No.: US 2026/0218316 A1
(43) Pub. Date: Jul. 30, 2026

(54) DIAGNOSTIC AND THERAPEUTIC METHODS FOR TREATING LUNG CANCER

(71) Applicant: Genentech, Inc., South San Francisco, CA (US)

(72) Inventors: Hartmut Karl-Wilhelm KOEPPEN, Berkeley, CA (US); Akshay Tilak KRISHNAMURTY, South San Francisco, CA (US); Soren MULLER, San Francisco, CA (US); Barzin Y. NABET, Bala Cynwyd, PA (US); Minu Kumari SRIVASTAVA, Fremont, CA (US)

(73) Assignee: Genentech, Inc., South San Francisco, CA (US)

(21) Appl. No.: 19/647,246

(22) Filed: Apr. 14, 2026

Related U.S. Application Data
(63) Continuation of application No. PCT/US2024/051343, filed on Oct. 15, 2024.
(60) Provisional application No. 63/590,753, filed on Oct. 16, 2023.

(51) Int. Cl. C12Q 1/6886; A61K 39/00; A61P 35/00; C07K 16/28; G01N 33/575

(52) U.S. Cl. CPC C12Q 1/6886; A61P 35/00; C07K 16/2827; G01N 33/5758; A61K 2039/505; C07K 2317/24; C12Q 2600/106; C12Q 2600/158; G01N 2474/20

(57) ABSTRACT

The invention provides diagnostic methods for identifying individuals having a lung cancer (e.g., NSCLC) suitable for treatment a treatment regimen that includes a PD-1 axis binding antagonist (e.g., atezolizumab) after adjuvant therapy (e.g., after a platinum-based adjuvant therapy) and therapeutic methods for treating lung cancer (e.g., NSCLC) in an individual identified as one who may benefit from treatment with a treatment regimen that includes a PD-1 axis binding antagonist (e.g., atezolizumab) to the individual following an adjuvant therapy (e.g., after a platinum-based adjuvant therapy). Also provided are compositions (e.g., a PD-1 axis binding antagonist (e.g., atezolizumab), pharmaceutical compositions thereof, assays thereof, kits thereof, and articles of manufacture thereof) for use in identifying patients suitable for treatment and for treating lung cancer (e.g., NSCLC) in an identified individual.

### CROSS REFERENCE TO RELATED APPLICATIONS

This application is a continuation of P.C.T Application No. PCT/US2024/051343, filed Oct. 15, 2024, which claims priority to U.S. Application No. 63/590,753 filed Oct. 16, 2023, which are incorporated herein by reference in their entirety.

### SEQUENCE LISTING

The instant application contains a Sequence Listing which has been submitted electronically in XML format and is hereby incorporated by reference in its entirety. Said XML copy, created on Oct. 15, 2024, is named “50474-342WO2_Sequence_Listing_10_15_24.xml” and is 10,409 bytes in size.

### FIELD OF THE INVENTION

This invention relates to methods and compositions for use in treating lung cancer (e.g., non-small cell lung cancer (NSCLC)) in an individual, for example, by administering to the individual a treatment regimen that includes a PD-1 axis binding antagonist (e.g., atezolizumab) following an adjuvant therapy.

### BACKGROUND OF THE INVENTION

Treatment with anti-PD-1 and anti-PD-L1 inhibitors has transformed the treatment of non-oncogenic non-small cell lung cancer (NSCLC), (Mohamed S. et al, Cancers 2023; 15 (9): 2476; Planchard D et al., Annals of Oncology 2018; 29 (Supplement_4): iv192-iv237; Hanna N H, Schneider B J, Temin S et al. Journal of Clinical Oncology 2020; 38 (14): 1608-1632) but despite these advances, clinical responses are generally only observed in a subset of treated individuals (Massafra M, Passalacqua M I, Gebbia V et al., Biologics: Targets and Therapy 2021; Volume 15:399-417). Biomarkers such as PD-L1, tumor mutation burden, tumor-infiltrating lymphocytes, and tumor-specific genotypes have provided insight into NSCLC disease prognosis and predictive response to immune checkpoint inhibitors (Bodor J N et al., Cancer 2020; 126 (2): 260-270).

RNA seq is a powerful tool to identify gene signatures underlying tumor microenvironment, cell biology, immune biology or predictive biomarkers that associate with the treatment response or novel targets for the treatment of NSCLC (Xue Q et al., Front. Immunol. 2023; 14:1148061). Understanding of the predictive response to immunotherapy may identify individuals most likely to respond to immunotherapy and help guide treatment decisions.

### SUMMARY OF THE INVENTION

The invention provides, inter alia, methods of treating lung cancer (e.g., NSCLC), as well as related compositions for use, uses, kits, and articles of manufacture. In one aspect, the invention features a method of adjuvant treatment following resection and platinum-based chemotherapy for an adult individual with stage IB to IIIA NSCLC (e.g., stage II to IIIA NSCLC), the method comprising administering to the individual a treatment regimen comprising an effective amount of atezolizumab, wherein the treatment regimen extends the individual's disease-free survival (DFS) and/or overall survival (OS) as compared to best supportive care, and wherein a tumor tissue sample obtained from the individual has been determined to have an expression level of an TGFβ CAF gene signature that is at or above a reference TGFβ CAF gene signature expression level and/or a tumor tissue sample from the individual has been determined to have an expression level of LRRC15 that is at or above a reference LRRC15 expression level.

In one aspect, the invention provides a method for treating a non-small cell lung cancer (NSCLC) in an individual, the method comprising: (a) determining the expression level of a transforming growth factor-beta (TGFβ) cancer-associated fibroblast (CAF) gene signature in a tumor tissue sample obtained from the individual, wherein an expression level of the TGFβ CAF gene signature has been determined to be at or above a reference TGFβ CAF gene signature expression level, thereby indicating that the individual is likely to have an increased clinical benefit from an adjuvant treatment comprising the anti-PD-L1 antibody compared to treatment with best supportive care (BSC); and (b) administering an effective amount of the anti-PD-L1 antibody to the individual, wherein the anti-PD-L1 antibody comprises the following hypervariable regions (HVRs): (i) an HVR-H1 sequence of GFTFSDSWIH (SEQ ID NO: 3); (ii) an HVR-H2 sequence of AWISPYGGSTYYADSVKG (SEQ ID NO: 4); (iii) an HVR-H3 sequence of RHWPGGFDY (SEQ ID NO: 5); (iv) an HVR-L1 sequence of RASQDVSTAVA (SEQ ID NO: 6); (v) an HVR-L2 sequence of SASFLYS (SEQ ID NO: 7); and (vi) an HVR-L3 sequence of QQYLYHPAT (SEQ ID NO: 8).

In one aspect, the invention provides a method of treating an individual having a NSCLC, the method comprising administering to the individual an effective amount of an anti-PD-L1 antibody, wherein a tumor tissue sample from the individual has been determined to have an expression level of a TGFβ CAF gene signature that is at or above a reference TGFβ CAF gene signature expression level, thereby indicating that the individual is likely to have an increased clinical benefit from an adjuvant treatment comprising the anti-PD-L1 antibody compared to treatment with BSC, and wherein the anti-PD-L1 antibody comprises the following HVRs: (i) an HVR-H1 sequence of GFTFSDSWIH (SEQ ID NO: 3); (ii) an HVR-H2 sequence of AWISPYGGSTYYADSVKG (SEQ ID NO: 4); (iii) an HVR-H3 sequence of RHWPGGFDY (SEQ ID NO: 5); (iv) an HVR-L1 sequence of RASQDVSTAVA (SEQ ID NO: 6); (v) an HVR-L2 sequence of SASFLYS (SEQ ID NO: 7); and (vi) an HVR-L3 sequence of QQYLYHPAT (SEQ ID NO: 8).

In one aspect, the invention provides a method of identifying an individual having a NSCLC who is likely to have an increased clinical benefit from an adjuvant treatment comprising an anti-PD-L1 antibody compared to treatment with BSC, the method comprising determining the expression level of a TGFβ CAF gene signature in a tumor tissue sample obtained from the individual, wherein an expression level of the TGFβ CAF gene signature has been determined to be at or above a reference TGFβ CAF gene signature expression level, thereby indicating that the individual is likely to have an increased clinical benefit from the adjuvant treatment comprising the anti-PD-L1 antibody compared to treatment with BSC, wherein the anti-PD-L1 antibody comprises the following HVRs: (i) an HVR-H1 sequence of GFTFSDSWIH (SEQ ID NO: 3); (ii) an HVR-H2 sequence of AWISPYGGSTYYADSVKG (SEQ ID NO: 4); (iii) an HVR-H3 sequence of RHWPGGFDY (SEQ ID NO: 5); (iv) an HVR-L1 sequence of RASQDVSTAVA (SEQ ID NO: 6); (v) an HVR-L2 sequence of SASFLYS (SEQ ID NO: 7); and (vi) an HVR-L3 sequence of QQYLYHPAT (SEQ ID NO: 8).

In one aspect, the invention provides a method for selecting a therapy for an individual having a NSCLC, the method comprising determining the expression level of a TGFβ CAF gene signature in a tumor tissue sample obtained from the individual, wherein an expression level of the TGFβ CAF gene signature has been determined to be at or above a reference TGFβ CAF gene signature expression level, thereby indicating that the individual is likely to have an increased clinical benefit from an adjuvant treatment comprising the anti-PD-L1 antibody compared to treatment with BSC, wherein the anti-PD-L1 antibody comprises the following HVRs: (i) an HVR-H1 sequence of GFTFSDSWIH (SEQ ID NO: 3); (ii) an HVR-H2 sequence of AWISPYGGSTYYADSVKG (SEQ ID NO: 4); (iii) an HVR-H3 sequence of RHWPGGFDY (SEQ ID NO: 5); (iv) an HVR-L1 sequence of RASQDVSTAVA (SEQ ID NO: 6); (v) an HVR-L2 sequence of SASFLYS (SEQ ID NO: 7); and (vi) an HVR-L3 sequence of QQYLYHPAT (SEQ ID NO: 8).

In some embodiments, the method further comprises administering to the individual an effective amount of the anti-PD-L1 antibody.

In one aspect, the invention provides a kit for identifying an individual having a NSCLC who is likely to have an increased clinical benefit from an adjuvant treatment comprising an anti-PD-L1 antibody compared to treatment with BSC, the kit comprising reagents for determining the expression level of a TGFβ CAF gene signature in a tumor tissue sample obtained from the individual, wherein an expression level of a TGFβ CAF gene signature that is at or above a reference TGFβ CAF gene signature expression level identifies that the individual as one who is likely to have an increased clinical benefit from the adjuvant treatment comprising the anti-PD-L1 antibody compared to treatment with BSC, wherein the anti-PD-L1 antibody comprises the following HVRs: (i) an HVR-H1 sequence of GFTFSDSWIH (SEQ ID NO: 3); (ii) an HVR-H2 sequence of AWISPYGGSTYYADSVKG (SEQ ID NO: 4); (iii) an HVR-H3 sequence of RHWPGGFDY (SEQ ID NO: 5); (iv) an HVR-L1 sequence of RASQDVSTAVA (SEQ ID NO: 6); (v) an HVR-L2 sequence of SASFLYS (SEQ ID NO: 7); and (vi) an HVR-L3 sequence of QQYLYHPAT (SEQ ID NO: 8).

In one aspect, the invention provides an assay for identifying an individual having a NSCLC who is a candidate for an adjuvant treatment comprising an anti-PD-L1 antibody, the assay comprising determining the expression level of a TGFβ CAF gene signature in a tumor tissue sample obtained from the individual, wherein an expression level of the TGFβ CAF gene signature has been determined to be at or above a reference TGFβ CAF gene signature expression level, thereby indicating that the individual is likely to have an increased clinical benefit from the adjuvant treatment comprising the anti-PD-L1 antibody compared to treatment with BSC, wherein the anti-PD-L1 antibody comprises the following HVRs: (i) an HVR-H1 sequence of GFTFSDSWIH (SEQ ID NO: 3); (ii) an HVR-H2 sequence of AWISPYGGSTYYADSVKG (SEQ ID NO: 4); (iii) an HVR-H3 sequence of RHWPGGFDY (SEQ ID NO: 5); (iv) an HVR-L1 sequence of RASQDVSTAVA (SEQ ID NO: 6); (v) an HVR-L2 sequence of SASFLYS (SEQ ID NO: 7); and (vi) an HVR-L3 sequence of QQYLYHPAT (SEQ ID NO: 8).

In one aspect, the invention provides a method for treating a NSCLC in an individual, the method comprising: (a) determining the expression level of LRRC15 in a tumor tissue sample obtained from the individual, wherein an expression level of LRRC15 has been determined to be at or above a reference LRRC15 expression level, thereby indicating that the individual is likely to have an increased clinical benefit from an adjuvant treatment comprising the anti-PD-L1 antibody compared to treatment with BSC; and (b) administering an effective amount of the anti-PD-L1 antibody to the individual, wherein the anti-PD-L1 antibody comprises the following hypervariable regions (HVRs): (i) an HVR-H1 sequence of GFTFSDSWIH (SEQ ID NO: 3); (ii) an HVR-H2 sequence of AWISPYGGSTYYADSVKG (SEQ ID NO: 4); (iii) an HVR-H3 sequence of RHWPGGFDY (SEQ ID NO: 5); (iv) an HVR-L1 sequence of RASQDVSTAVA (SEQ ID NO: 6); (v) an HVR-L2 sequence of SASFLYS (SEQ ID NO: 7); and (vi) an HVR-L3 sequence of QQYLYHPAT (SEQ ID NO: 8).

In one aspect, the invention provides a method of treating an individual having a NSCLC, the method comprising administering to the individual an effective amount of an anti-PD-L1 antibody, wherein a tumor tissue sample from the individual has been determined to have an expression level of LRRC15 that is at or above a reference LRRC15 expression level, thereby indicating that the individual is likely to have an increased clinical benefit from an adjuvant treatment comprising the anti-PD-L1 antibody compared to treatment with BSC, and wherein the anti-PD-L1 antibody comprises the following HVRs: (i) an HVR-H1 sequence of GFTFSDSWIH (SEQ ID NO: 3); (ii) an HVR-H2 sequence of AWISPYGGSTYYADSVKG (SEQ ID NO: 4); (iii) an HVR-H3 sequence of RHWPGGFDY (SEQ ID NO: 5); (iv) an HVR-L1 sequence of RASQDVSTAVA (SEQ ID NO: 6); (v) an HVR-L2 sequence of SASFLYS (SEQ ID NO: 7); and (vi) an HVR-L3 sequence of QQYLYHPAT (SEQ ID NO: 8).

In one aspect, the invention provides a method of identifying an individual having a NSCLC who is likely to have an increased clinical benefit from an adjuvant treatment comprising an anti-PD-L1 antibody compared to treatment with BSC, the method comprising determining the expression level of LRRC15 in a tumor tissue sample obtained from the individual, wherein an expression level of LRRC15 has been determined to be at or above a reference LRRC15 expression level, thereby indicating that the individual is likely to have an increased clinical benefit from the adjuvant treatment comprising the anti-PD-L1 antibody compared to treatment with BSC, wherein the anti-PD-L1 antibody comprises the following HVRs: (i) an HVR-H1 sequence of GFTFSDSWIH (SEQ ID NO: 3); (ii) an HVR-H2 sequence of AWISPYGGSTYYADSVKG (SEQ ID NO: 4); (iii) an HVR-H3 sequence of RHWPGGFDY (SEQ ID NO: 5); (iv) an HVR-L1 sequence of RASQDVSTAVA (SEQ ID NO: 6); (v) an HVR-L2 sequence of SASFLYS (SEQ ID NO: 7); and (vi) an HVR-L3 sequence of QQYLYHPAT (SEQ ID NO: 8).

In one aspect, the invention provides a method for selecting a therapy for an individual having a NSCLC, the method comprising determining the expression level of LRRC15 in a tumor tissue sample obtained from the individual, wherein an expression level of LRRC15 has been determined to be at or above a reference LRRC15 expression level, thereby indicating that the individual is likely to have an increased clinical benefit from an adjuvant treatment comprising the anti-PD-L1 antibody compared to treatment with BSC, wherein the anti-PD-L1 antibody comprises the following HVRs: (i) an HVR-H1 sequence of GFTFSDSWIH (SEQ ID NO: 3); (ii) an HVR-H2 sequence of AWISPYGGSTYYADSVKG (SEQ ID NO: 4); (iii) an HVR-H3 sequence of RHWPGGFDY (SEQ ID NO: 5); (iv) an HVR-L1 sequence of RASQDVSTAVA (SEQ ID NO: 6); (v) an HVR-L2 sequence of SASFLYS (SEQ ID NO: 7); and (vi) an HVR-L3 sequence of QQYLYHPAT (SEQ ID NO: 8).

In some embodiments, the method further comprises administering to the individual an effective amount of the anti-PD-L1 antibody.

In one aspect, the invention provides a kit for identifying an individual having a NSCLC who is likely to have an increased clinical benefit from an adjuvant treatment comprising an anti-PD-L1 antibody compared to treatment with BSC, the kit comprising reagents for determining the expression level of LRRC15 in a tumor tissue sample obtained from the individual, wherein an expression level of LRRC15 that is at or above a reference LRRC15 expression level identifies that the individual as one who is likely to have an increased clinical benefit from the adjuvant treatment comprising the anti-PD-L1 antibody compared to treatment with BSC, wherein the anti-PD-L1 antibody comprises the following HVRs: (i) an HVR-H1 sequence of GFTFSDSWIH (SEQ ID NO: 3); (ii) an HVR-H2 sequence of AWISPYGGSTYYADSVKG (SEQ ID NO: 4); (iii) an HVR-H3 sequence of RHWPGGFDY (SEQ ID NO: 5); (iv) an HVR-L1 sequence of RASQDVSTAVA (SEQ ID NO: 6); (v) an HVR-L2 sequence of SASFLYS (SEQ ID NO: 7); and (vi) an HVR-L3 sequence of QQYLYHPAT (SEQ ID NO: 8).

In one aspect, the invention provides an assay for identifying an individual having a NSCLC who is a candidate for an adjuvant treatment comprising an anti-PD-L1 antibody, the assay comprising determining the expression level of LRRC15 in a tumor tissue sample obtained from the individual, wherein an expression level of LRRC15 has been determined to be at or above a reference LRRC15 expression level, thereby indicating that the individual is likely to have an increased clinical benefit from the adjuvant treatment comprising the anti-PD-L1 antibody compared to treatment with BSC, wherein the anti-PD-L1 antibody comprises the following HVRs: (i) an HVR-H1 sequence of GFTFSDSWIH (SEQ ID NO: 3); (ii) an HVR-H2 sequence of AWISPYGGSTYYADSVKG (SEQ ID NO: 4); (iii) an HVR-H3 sequence of RHWPGGFDY (SEQ ID NO: 5); (iv) an HVR-L1 sequence of RASQDVSTAVA (SEQ ID NO: 6); (v) an HVR-L2 sequence of SASFLYS (SEQ ID NO: 7); and (vi) an HVR-L3 sequence of QQYLYHPAT (SEQ ID NO: 8).

In some embodiments, the anti-PD-L1 antibody comprises:
(a) a VH comprising the amino acid sequence: EVOLVESGGGLVQPGGSLRLSCAASGFTFSDSWIHWVRQAPGKGLEWVAWISPYGGSTYYADSVKGRF TISADTSKNTAYLQMNSLRAEDTAVYYCARRHWPGGFDYWGQGTLVTVSS (SEQ ID NO: 9), and
(b) a VL comprising the amino acid sequence:

(SEQ ID NO: 10)
DIQMTQSPSSLSASVGDRVTITCRASQDVSTAVAWYQQKPGKAPKLLIYSA
SFLYSGVPSRFSGSGSGTDFTLTISSLQPEDFATYYCQQYLYHPATFGQGT
KVEIKR.

In some embodiments, the anti-PD-L1 antibody comprises:
(a) a heavy chain comprising amino acid sequence: EVOLVESGGGLVQPGGSLRLSCAASGFTFSDSWIHWVRQAPGKGLEWVAWISPYGGSTYYADSVKGRF TISADTSKNTAYLQMNSLRAEDTAVYYCARRHWPGGFDYWGQGTLVTVSSASTKGPSVFPLAPSSKSTS GGTAALGCLVKDYFPEPVTVSWNSGALTSGVHTFPAVLQSSGLYSLSSVVTVPSSSLGTQTYICNVNHKP SNTKVDKKVEPKSCDKTHTCPPCPAPELLGGPSVFLFPPKPKDTLMISRTPEVTCVVVDVSHEDPEVKFN WYVDGVEVHNAKTKPREEQYASTYRVVSVLTVLHQDWLNGKEYKCKVSNKALPAPIEKTISKAKGQPRE PQVYTLPPSREEMTKNQVSLTCLVKGFYPSDIAVEWESNGQPENNYKTTPPVLDSDGSFFLYSKLTVDKS RWQQGNVFSCSVMHEALHNHYTQKSLSLSPG (SEQ ID NO: 1), and
(b) a light chain comprising the amino acid sequence:

(SEQ ID NO: 2)
DIQMTQSPSSLSASVGDRVTITCRASQDVSTAVAWYQQKPGKAPKLLIYSA
SFLYSGVPSRFSGSGSGTDFTLTISSLQPEDFATYYCQQYLYHPATFGQGT
KVEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSP
VTKSFNRGEC.

In some embodiments, the anti-PD-L1 antibody is atezolizumab.

In some embodiments, the adjuvant treatment comprises intravenous or subcutaneous administration of the anti-PD-L1 antibody to the individual.

In some embodiments, the adjuvant treatment comprises administration of the anti-PD-L1 antibody as a monotherapy. In some embodiments, the adjuvant treatment comprises intravenous administration of the anti-PD-L1 antibody to the individual at a dose of 1200 mg every 3 weeks. In some embodiments, the adjuvant treatment comprises subcutaneous administration of the anti-PD-L1 antibody to the individual at a dose of 1875 mg every 3 weeks. In some embodiments, the adjuvant treatment comprises administration of the anti-PD-L1 antibody for up to 1 year.

In some embodiments, the individual has previously received a platinum-based adjuvant therapy.

In some embodiments, the platinum-based adjuvant therapy comprises cisplatin. In some embodiments, the platinum-based adjuvant therapy further comprises one or more additional chemotherapeutic agents. In some embodiments, the one or more additional chemotherapeutic agents comprises a vinca alkaloid, a taxane, an anti-metabolite, or a combination thereof. In some embodiments, the vinca alkaloid is vinorelbine. In some embodiments, the taxane is docetaxel. In some embodiments, the anti-metabolite is gemcitabine or pemetrexed. In some embodiments, the anti-metabolite is gemcitabine. In some embodiments, the anti-metabolite is pemetrexed.

In some embodiments, the platinum-based adjuvant therapy comprises up to four cycles. In some embodiments, the platinum-based adjuvant therapy comprises up to four 21-day cycles. In some embodiments, the platinum-based adjuvant therapy comprises cisplatin, wherein cisplatin was administered at a dose of about 75 mg/m² intravenously on Day 1 of each 21-day cycle. In some embodiments, the platinum-based adjuvant therapy comprises cisplatin and vinorelbine, wherein cisplatin was administered at a dose of about 75 mg/m² intravenously on Day 1 of each 21 day-cycle, and vinorelbine was administered at a dose of about 30 mg/m² by intravenous push on Days 1 and 8 of each 21-day cycle. In some embodiments, the platinum-based adjuvant therapy comprises cisplatin and docetaxel, wherein cisplatin was administered at a dose of about 75 mg/m² intravenously on Day 1 of each 21 day-cycle, and docetaxel was administered at a dose of about 75 mg/m² intravenously on Day 1 of each 21-day cycle. In some embodiments, the platinum-based adjuvant therapy comprises cisplatin and gemcitabine, wherein cisplatin was administered at a dose of about 75 mg/m² intravenously on Day 1 of each 21 day-cycle, and gemcitabine was administered at a dose of about 1250 mg/m² intravenously on Days 1 and 8 of each 21-day cycle. In some embodiments, the platinum-based adjuvant therapy comprises cisplatin and pemetrexed, wherein cisplatin was administered at a dose of about 75 mg/m² intravenously on Day 1 of each 21 day-cycle, and pemetrexed was administered at a dose of about 500 mg/m² intravenously on Day 1 of each 21-day cycle.

In some embodiments, the NSCLC is stage IB, IIA, IIB, or IIIA. In some embodiments, the NSCLC staging is per the Union Internationale Contre le Cancer/American Joint Committee on Cancer (UICC/AJCC) staging system, 7th edition. In some embodiments, the NSCLC is not an EGFR/ALK+NSCLC. In some embodiments, the NSCLC is squamous or non-squamous NSCLC. In some embodiments, the NSCLC is squamous NSCLC. In some embodiments, the NSCLC is non-squamous NSCLC.

In some embodiments, the individual had a complete resection of the NSCLC prior to the platinum-based adjuvant therapy. In some embodiments, the complete resection comprises lobectomy, sleeve lobectomy, bilobectomy, or pneumonectomy. In some embodiments, the individual had complete resection of the NSCLC about 4 to about 12 weeks prior to the platinum-based adjuvant therapy. In some embodiments, the individual has not had treatment with systemic chemotherapy prior to the platinum-based adjuvant therapy.

In some embodiments, the increased clinical benefit is an increase in disease-free survival (DFS).

In some embodiments, the increased clinical benefit is an increase in overall survival (OS).

In some embodiments, the tumor tissue sample is obtained from the individual prior to administration of the anti-PD-L1 antibody. In some embodiments, the tumor tissue sample is obtained from the individual prior to administration of a platinum-based adjuvant therapy.

In some embodiments, the reference TGFβ CAF gene signature expression level is the median expression level of the TGFβ CAF gene signature in a reference population.

In some embodiments, the expression level of the TGFβ CAF gene signature is the median or median of the expression levels of a set of genes comprising the TGFβ CAF gene signature.

In some embodiments, the set of genes comprising the TGFβ CAF gene signature comprises the following genes: LRRC15, FAP, COL11A1, COL10A1, FNDC1, GREM1, ITGA11, WISP1, PLPP4, PODNL1, TWIST1, COL8A2, SUGCT, NOX4, ZNF469, TENM3, P4HA3, ADAMTS12, and CPZ. In some embodiments, the set of genes comprising the TGFβ CAF gene signature consists of LRRC15, FAP, COL11A1, COL10A1, FNDC1, GREM1, ITGA11, WISP1, PLPP4, PODNL1, TWIST1, COL8A2, SUGCT, NOX4, ZNF469, TENM3, P4HA3, ADAMTS12, and CPZ.

In some embodiments, the reference expression level of LRRC15 is the median expression level of LRRC15 in a reference population.

In some embodiments, the reference population comprises a set of individuals having the NSCLC.

In some embodiments, the expression level is an RNA expression level. In some embodiments, the RNA expression level is determined using RNA sequencing. In some embodiments, the RNA sequencing is performed using an Illumina sequencing-by-synthesis platform. In some embodiments, the Illumina sequencing-by-synthesis platform is a NOVASEQ® 6000. In some embodiments, the RNA sequencing is performed on a library generated using a RIBO-ZERO® Magnetic Gold Kit.

In some embodiments, the expression level of LRRC15 is a protein expression level. In some embodiments, the protein expression level is determined using immunohistochemistry (IHC). In some embodiments, the IHC is performed using a Ventana staining platform.

In one aspect, the invention provides a pharmaceutical composition comprising an anti-PD-L1 antibody for use in an adjuvant treatment of an individual having an NSCLC, wherein the adjuvant treatment comprises administration of an effective amount of the anti-PD-L1 antibody, wherein a tumor tissue sample obtained from the individual has been determined to have an expression level of an TGFβ CAF gene signature that is at or above a reference TGFβ CAF gene signature expression level, thereby indicating that the individual is likely to have an increased clinical benefit from treatment with the anti-PD-L1 antibody compared to treatment with BSC, and wherein the anti-PD-L1 antibody comprises the following HVRs: (i) an HVR-H1 sequence of GFTFSDSWIH (SEQ ID NO: 3); (ii) an HVR-H2 sequence of AWISPYGGSTYYADSVKG (SEQ ID NO: 4); (iii) an HVR-H3 sequence of RHWPGGFDY (SEQ ID NO: 5); (iv) an HVR-L1 sequence of RASQDVSTAVA (SEQ ID NO: 6); (v) an HVR-L2 sequence of SASFLYS (SEQ ID NO: 7); and (vi) an HVR-L3 sequence of QQYLYHPAT (SEQ ID NO: 8).

In one aspect, the invention provides use of atezolizumab in the manufacture of a medicament for adjuvant treatment of an individual having an NSCLC, wherein the adjuvant treatment comprises administration of an effective amount of the anti-PD-L1 antibody, wherein a tumor tissue sample obtained from the individual has been determined to have an expression level of an TGFβ CAF gene signature that is at or above a reference TGFβ CAF gene signature expression level, thereby indicating that the individual is likely to have an increased clinical benefit from treatment with the anti-PD-L1 antibody compared to treatment with BSC, and wherein the anti-PD-L1 antibody comprises the following HVRs: (i) an HVR-H1 sequence of GFTFSDSWIH (SEQ ID NO: 3); (ii) an HVR-H2 sequence of AWISPYGGSTYYADSVKG (SEQ ID NO: 4); (iii) an HVR-H3 sequence of RHWPGGFDY (SEQ ID NO: 5); (iv) an HVR-L1 sequence of RASQDVSTAVA (SEQ ID NO: 6); (v) an HVR-L2 sequence of SASFLYS (SEQ ID NO: 7); and (vi) an HVR-L3 sequence of QQYLYHPAT (SEQ ID NO: 8).

In one aspect, the invention provides an article of manufacture comprising an anti-PD-L1 antibody and instructions to administer the anti-PD-L1 antibody for adjuvant treatment of an individual having an NSCLC, wherein the adjuvant treatment comprises administration of an effective amount of the anti-PD-L1 antibody, wherein a tumor tissue sample obtained from the individual has been determined to have an expression level of an TGFβ CAF gene signature that is at or above a reference TGFβ CAF gene signature expression level, thereby indicating that the individual is likely to have an increased clinical benefit from the adjuvant treatment comprising the anti-PD-L1 antibody compared to treatment with BSC, and wherein the anti-PD-L1 antibody comprises the following HVRs: (i) an HVR-H1 sequence of GFTFSDSWIH (SEQ ID NO: 3); (ii) an HVR-H2 sequence of AWISPYGGSTYYADSVKG (SEQ ID NO: 4); (iii) an HVR-H3 sequence of RHWPGGFDY (SEQ ID NO: 5); (iv) an HVR-L1 sequence of RASQDVSTAVA (SEQ ID NO: 6); (v) an HVR-L2 sequence of SASFLYS (SEQ ID NO: 7); and (vi) an HVR-L3 sequence of QQYLYHPAT (SEQ ID NO: 8).

In one aspect, the invention provides a method of treating an individual comprising administering to the individual an adjuvant treatment comprising an effective amount of atezolizumab following complete resection and no progression after platinum-based chemotherapy for an individual with stage IB to IIIA NSCLC, wherein a tumor tissue sample obtained from the individual has been determined to have an expression level of an TGFβ CAF gene signature that is at or above a reference TGFβ CAF gene signature expression level, thereby indicating that the individual is likely to have an increased DFS and/or OS after the adjuvant treatment comprising the atezolizumab compared to treatment with BSC, wherein the expression level of the TGFβ CAF gene signature is the mean RNA expression level of a set of genes comprising LRRC15, FAP, COL11A1, COL10A1, FNDC1, GREM1, ITGA11, WISP1, PLPP4, PODNL1, TWIST1, COL8A2, SUGCT, NOX4, ZNF469, TENM3, P4HA3, ADAMTS12, and CPZ, and wherein the reference TGFβ CAF gene signature expression level is the median expression level of the TGFβ CAF gene signature in a reference population of individuals having stage IB to IIIA NSCLC.

In one aspect, the invention provides a method of treating an individual comprising administering to the individual an adjuvant treatment comprising an effective amount of atezolizumab following complete resection and no progression after platinum-based chemotherapy for an individual with stage IB to IIIA NSCLC, wherein the tumor tissue sample from the individual has been determined to have an RNA expression level of LRRC15 that is at or above a median RNA expression level of LRRC15 in a reference population of individuals having stage IB to IIIA NSCLC, thereby indicating that the individual is likely to have an increased DFS and/or OS after adjuvant treatment comprising the atezolizumab compared to treatment with BSC.

### BRIEF DESCRIPTION OF THE DRAWINGS

FIGS. 1A-1C are graphs showing disease-free survival (DFS; months) for the RNA-seq biomarker evaluable population (BEP) for individuals with completely resected stage IB-IIIA NSCLC who received adjuvant atezolizumab vs best supportive care (BSC) after platinum-based chemotherapy. Kaplan-Meier disease-free survival curve for the RNA-seq BEP (FIG. 1A), as well as the PD-L1 negative (FIG. 1B) and PD-L1 positive (FIG. 1C) subgroups of the RNA-seq BEP.

FIG. 2 is a graph showing cell signatures sorted by variable importance for a predictive association of DFS benefit with atezolizumab vs BSC.

FIG. 3 is a graph showing a subset of the cell signatures with highest importance for a predictive association of DFS benefit with atezolizumab vs BSC. EMT: Epithelial-Mesenchymal Transition-Based Gene Signature.

FIGS. 4A-4B are graphs showing DFS (months) for TGFβ-CAF (TGFb CAF) subgroups for individuals with completely resected stage IB-IIIA NSCLC who received adjuvant atezolizumab vs BSC after platinum-based chemotherapy. Kaplan-Meier DFS curves for the TGFβ CAF-low (FIG. 4A) and TGFβ CAF-high (FIG. 4B) subgroups.

FIGS. 5A-5D are graphs showing DFS (months) for TGFβ populations by histological subtype for individuals with completely resected stage IB-IIIA NSCLC who received adjuvant atezolizumab (atezo) vs BSC after platinum-based chemotherapy and tables quantifying median DFS (mDFS) and hazard ratio (HR) between atezolizumab vs BSC. Kaplan-Meier DFS curve for the TGFβ CAF-low population with squamous (FIG. 5A) or non-squamous (FIG. 5B) histology. Kaplan-Meier DFS curve for the TGFβ CAF-high population with squamous (FIG. 5C) or non-squamous (FIG. 5D) histology.

FIGS. 6A-6D are graphs showing DFS (months) for TGFβ CAF populations by PD-L1 expression status for individuals with completely resected stage IB-IIIA NSCLC who received adjuvant atezolizumab vs BSC after platinum-based chemotherapy and tables quantifying median DFS (mDFS) and hazard ratio (HR) between atezolizumab vs BSC. Kaplan-Meier DFS curve for the TGFβ CAF-low population with PD-L1-negative (FIG. 6A) or PD-L1-positive (FIG. 6B) tumors. Kaplan-Meier DFS curve for the TGFβ CAF-high population with PD-L1-negative (FIG. 6C) or PD-L1-positive (FIG. 6D) tumors. Tumors were defined as PD-L1 negative (PD-L1 TC<1%) or PD-L1 positive (PD-L1 TC ≥1%) by the SP263 immunohistochemistry assay (i.e., Ventana SP263 PD-L1 assay). TC, tumor cell.

FIG. 7 is a graph showing Epithelial-Mesenchymal Transition (EMT) signature scores in TGFβ CAF-low and TGFβ CAF-high populations.

FIG. 8 is a flow chart showing the process for RNA-seq and gene signature development. DFS, disease-free survival; LDH, lactate dehydrogenase; NLR, neutrophil to lymphocyte ratio.

FIG. 9 is a diagram depicting an overview of the IMpower010 (NCT02486718) clinical study.

FIGS. 10A-10D are Kaplan-Meier curves showing DFS (months) of the total population of Stage II-IIIA patients (FIG. 10A) or in BEP patients (FIG. 10B) or overall survival (OS) (months) in the total population of Stage II-IIIA patients (FIG. 10C) or in BEP patients (FIG. 10D) that were treated with atezolizumab or BSC.

FIGS. 11A-11B are forest plots showing the hazard ratios and median (months) of DFS (FIG. 11A) or OS (FIG. 11B) for treatment with atezolizumab or BSC for the total BEP population and each subgroup of patients with the listed RNA-sequencing and histology scoring features. nonSCC, non-squamous cell carcinoma; SCC, squamous cell carcinoma.

FIG. 12 is a heatmap of Z-scored genes corresponding to cell-intrinsic and tumor microenvironment features, in which each column represents a unique patient sample, and the samples are grouped and hierarchically clustered within each PD-L1 TC expression subset.

FIGS. 13A-13C are plots showing differential gene expression. Shown are volcano plots depicting differentially expressed genes in PD-L1-negative and PD-L1 TC ≥50% samples (FIG. 13A) or in PD-L1 TC 1-49% and PD-L1 TC ≥50% samples (FIG. 13B). Genes corresponding to CD274 (or PD-L1) and CD8A, CXCL9, CXCL10, GZMA, GZMB, IFNG, PRF1, and TBX21, which comprise the T-effector gene signature, are highlighted. Also shown is a box- and -whisker plot showing differential gene expression of the T-effector signature in PD-L1-negative, PD-L1 TC 1-49%, and PD-L1 TC ≥50% samples (FIG. 13C).

FIG. 14 is a Kaplan-Meier curve showing DFS (months) of patients treated with atezolizumab or BSC in the high T-effector gene expression subgroup.

FIGS. 15A-15B are graphs depicting differentially expressed gene signatures in long (>36 months) and short (<12 months) DFS in the atezolizumab arm (FIG. 15A) or the BSC arm (FIG. 15B).

FIGS. 16A-16D show expression data from RNA-seq analyses of CAFs and cancer-associated pericytes (CAPs). FIG. 16A is a schematic depicting the identification of genes specific for CAFs and CAPs. FIG. 16B is a UMAP projection that represents cells obtained from 30 patients, in which the UMAP representation is encircled by a ring that depicts the relative proportion of cells from each patient within the indicated cell type, and the relative proportion of the indicated cell types obtained from a core biopsy, a resection, or a bronchoscopy, in which the relative expression level of the indicated gene in the represented cell types obtained from the patients is also shown. FIG. 16C is series of graphs showing a UMAP projection that represents gene clusters corresponding to CAF and CAP cell types, a heatmap showing the relative expression of the indicated genes of the corresponding cell type, and graphs that depict the distribution of relative expression of the indicated gene in each cell type. FIG. 16D is a series of UMAP projections for each type of CAF or CAP with a graph below each projection that shows the distribution of the CAF/CAP gene signature scores across the CAF or CAP cell types and other cell types.

FIGS. 17A-17B are data showing the relationship of gene signatures and clinical benefit from treatment with atezolizumab or BSC. FIG. 17A is a forest plot showing the relative variable importance of expression of the indicated biomarkers or gene signatures and the clinical benefit from each treatment, and FIG. 17B is a graph showing the association of P-value for increased expression of various gene signatures and DFS in each treatment arm, in which the TGFβ CAF gene signature is highlighted.

FIGS. 18A-18D are Kaplan-Meier curves of outcomes of patients treated with atezolizumab or BSC according to TGFβ CAF subgroups. TGFβ CAF-high population DFS (months) (FIG. 18A) or OS (months) (FIG. 18B) are shown, and TGFβ CAF-low population DFS (months) (FIG. 18C) or OS (months) (FIG. 18D) are shown.

FIGS. 19A-19B are Kaplan-Meier curves showing DFS (months) (FIG. 19A) or OS (months (FIG. 19B) of patients for patients treated with BSC in the TGFβ CAF-high and TGFβ CAF-low subgroups.

FIG. 20 is a Kaplan-Meier curve of data from the Phase III OAK clinical study (NCT02008227), showing OS (months) of patients with NSCLC treated with atezolizumab or docetaxel in TGFβ CAF-high and TGFβ CAF-low subgroups.

FIG. 21 is a Kaplan-Meier curve of data from the Phase III IMvigor010 study (NCT02450331), showing OS (months) of patients with early-stage resectable urothelial cancer treated with adjuvant atezolizumab versus observation in TGFβ CAF-high and TGFβ CAF-low subgroups.

FIG. 22 is a graph depicting the linear correlation between each indicated biomarker.

FIGS. 23A-23B are box- and -whisker plots showing the EMT signature score (FIG. 23A) or the TBFβ response signature score (FIG. 23B) in the TGFβ CAF-high and TGFβ CAF-low subgroups.

FIG. 24 is a series of nested bar graphs showing the proportion of patients that had low or high expression of the TGFβ CAF gene signature and the indicated PD-L1 TC expression, high or low T-effector cell gene signature expression, and circulating tumor DNA (ctDNA) following tumor resection (post-surgery).

FIG. 25 is a bar graph showing the proportion of patients in the TGFβ CAF expression subgroups in each treatment arm that had distant lung cancer recurrence.

FIG. 26 is a forest plot showing the DFS hazard ratio and median DFS (months) for atezolizumab and BSC treatments in each indicated population and further delineated by TGFβ CAF expression subgroups.

FIGS. 27A-27C are Kaplan-Meier curves showing DFS (months) of patients treated with atezolizumab or BSC in the TGFβ CAF-high and PD-L1-negative subgroups (FIG. 27A), the TGFβ CAF-high and PD-L1 TC 1-49% subgroups (FIG. 27B), or the TGFβ CAF-high and PD-L1 TC ≥50% subgroups (FIG. 27C).

FIGS. 28A-28C are Kaplan-Meier curves showing OS (months) of patients treated with atezolizumab or BSC in the TGFβ CAF-high and PD-L1-negative subgroups (FIG. 28A), the TGFβ CAF-high and PD-L1 TC 1-49% subgroups (FIG. 28B), or the TGFβ CAF-high and PD-L1 TC ≥50% subgroups (FIG. 28C).

FIG. 29 is a forest plot showing the OS hazard ratios and median OS (months) for treatment with atezolizumab or BSC in each indicated population and further delineated by TGFβ CAF expression subgroups.

FIGS. 30A-30F are Kaplan-Meier curves of outcomes for patients in different subgroups treated with atezolizumab or BSC. DFS (months) in the TGFβ CAF-high and non-squamous population (FIG. 30A) and the TGFβ CAF-high and squamous population (FIG. 30B) is shown, and OS (months) in the TGFβ CAF-high and non-squamous population (FIG. 30C) and the TGFβ CAF-high and squamous population (FIG. 30D) are shown. Also shown are the DFS (months) (FIG. 30E) and OS (months (FIG. 30F) of the TGFβ CAF-low and PD-L1 TC ≥50% population.

FIGS. 31A-31B are Kaplan-Meier curves showing DFS (months) of patients treated with atezolizumab or BSC in the LRRC15-high subgroup (FIG. 31A) or the LRRC15-low subgroup (FIG. 31B).

FIGS. 32A-32D are related to LRRC15 protein expression data as determined by an immunohistochemistry and immunofluorescence (IHC/IF) assay. FIG. 32A is a series of representative micrographs in samples from LRRC15-high and LRRC15-low subgroups, in which the left micrograph of each subgroup is immunohistochemical detection of LRRC15, CD8, and CK expression, and the right micrograph of each subgroup is immunofluorescence detection of CK expression (scale bar is 50 μm). Shown are graphs depicting the association between LRRC15 signal in the tumor or stroma area determined by IHC/IF and the quantified LRRC15 RNA expression determined by RNA-seq (FIG. 32B) or the quantified CD8 cells determined by IHC/IF (FIG. 32C). Also shown is the proportion of patients in the LRRC15-high and LRRC15-low subgroups as measured by IHC/IF categorized by PD-L1 TC expression levels (FIG. 32D).

FIG. 33 is a Kaplan-Meier curve showing DFS (months) in patients treated with atezolizumab or BSC in the LRRC15-high subgroup as determined by IHC/IF.

FIG. 34 is a graph comparing the relative proportion of patients in the PD-L1 TC subgroups determined by the SP263 IHC assay, as compared to the LRRC15-high or CD8-high subgroups determined by IHC/IF.

FIGS. 35A-35B are Kaplan-Meier curves showing DFS (months) (FIG. 35A) or OS (months) (FIG. 35B) in patients treated with atezolizumab or BSC in the LRRC15-high or CD8-high subgroups.

### DETAILED DESCRIPTION OF THE INVENTION

### I. Definitions

The term “about” as used herein refers to the usual error range for the respective value readily known to the skilled person in this technical field. Reference to “about” a value or parameter herein includes (and describes) embodiments that are directed to that value or parameter per se. In some instances, reference to “about” a value or parameter herein includes (and describes) embodiments that are directed to that value or parameter±10%.

The term “PD-1 axis binding antagonist” refers to a molecule that inhibits the interaction of a PD-1 axis binding partner with either one or more of its binding partners, so as to remove T-cell dysfunction resulting from signaling on the PD-1 signaling axis, with a result being to restore or enhance T-cell function (e.g., proliferation, cytokine production, and/or target cell killing). As used herein, a PD-1 axis binding antagonist includes a PD-L1 binding antagonist, a PD-1 binding antagonist, and a PD-L2 binding antagonist. In some instances, the PD-1 axis binding antagonist includes a PD-L1 binding antagonist or a PD-1 binding antagonist. In a preferred aspect, the PD-1 axis binding antagonist is a PD-L1 binding antagonist.

The term “PD-L1 binding antagonist” refers to a molecule that decreases, blocks, inhibits, abrogates, or interferes with signal transduction resulting from the interaction of PD-L1 with either one or more of its binding partners, such as PD-1 and/or B7-1. In some instances, a PD-L1 binding antagonist is a molecule that inhibits the binding of PD-L1 to its binding partners. In a specific aspect, the PD-L1 binding antagonist inhibits binding of PD-L1 to PD-1 and/or B7-1. In some instances, the PD-L1 binding antagonists include anti-PD-L1 antibodies, antigen-binding fragments thereof, immunoadhesins, fusion proteins, oligopeptides and other molecules that decrease, block, inhibit, abrogate or interfere with signal transduction resulting from the interaction of PD-L1 with one or more of its binding partners, such as PD-1 and/or B7-1. In one instance, a PD-L1 binding antagonist reduces the negative co-stimulatory signal mediated by or through cell surface proteins expressed on T lymphocytes mediated signaling through PD-L1 so as to render a dysfunctional T-cell less dysfunctional (e.g., enhancing effector responses to antigen recognition). In some instances, the PD-L1 binding antagonist binds to PD-L1. In some instances, a PD-L1 binding antagonist is an anti-PD-L1 antibody (e.g., an anti-PD-L1 antagonist antibody). Exemplary anti-PD-L1 antagonist antibodies include atezolizumab, MDX-1105, MEDI4736 (durvalumab), MSB0010718C (avelumab), SHR-1316, CS1001, envafolimab, TQB2450, ZKAB001, LP-002, CX-072, IMC-001, KL-A167, APL-502, cosibelimab, lodapolimab, FAZ053, TG-1501, BGB-A333, BCD-135, AK-106, LDP, GR1405, HLX20, MSB2311, RC98, PDL-GEX, KD036, KY1003, YBL-007, and HS-636. In some aspects, the anti-PD-L1 antibody is atezolizumab, MDX-1105, MEDI4736 (durvalumab), or MSB0010718C (avelumab). In one specific aspect, the PD-L1 binding antagonist is MDX-1105. In another specific aspect, the PD-L1 binding antagonist is MEDI4736 (durvalumab). In another specific aspect, the PD-L1 binding antagonist is MSB0010718C (avelumab). In other aspects, the PD-L1 binding antagonist may be a small molecule, e.g., GS-4224, INCB086550, MAX-10181, INCB090244, CA-170, or ABSK041, which in some instances may be administered orally. Other exemplary PD-L1 binding antagonists include AVA-004, MT-6035, VXM10, LYN192, GB7003, and JS-003. In a preferred aspect, the PD-L1 binding antagonist is atezolizumab.

The term “PD-1 binding antagonist” refers to a molecule that decreases, blocks, inhibits, abrogates or interferes with signal transduction resulting from the interaction of PD-1 with one or more of its binding partners, such as PD-L1 and/or PD-L2. PD-1 (programmed death 1) is also referred to in the art as “programmed cell death 1,” “PDCD1,” “CD279,” and “SLEB2.” An exemplary human PD-1 is shown in UniProtKB/Swiss-Prot Accession No. Q15116. In some instances, the PD-1 binding antagonist is a molecule that inhibits the binding of PD-1 to one or more of its binding partners. In a specific aspect, the PD-1 binding antagonist inhibits the binding of PD-1 to PD-L1 and/or PD-L2. For example, PD-1 binding antagonists include anti-PD-1 antibodies, antigen-binding fragments thereof, immunoadhesins, fusion proteins, oligopeptides, and other molecules that decrease, block, inhibit, abrogate or interfere with signal transduction resulting from the interaction of PD-1 with PD-L1 and/or PD-L2. In one instance, a PD-1 binding antagonist reduces the negative co-stimulatory signal mediated by or through cell surface proteins expressed on T lymphocytes mediated signaling through PD-1 so as render a dysfunctional T-cell less dysfunctional (e.g., enhancing effector responses to antigen recognition). In some instances, the PD-1 binding antagonist binds to PD-1. In some instances, the PD-1 binding antagonist is an anti-PD-1 antibody (e.g., an anti-PD-1 antagonist antibody). Exemplary anti-PD-1 antagonist antibodies include nivolumab, pembrolizumab, MEDI-0680, PDR001 (spartalizumab), REGN2810 (cemiplimab), BGB-108, prolgolimab, camrelizumab, sintilimab, tislelizumab, toripalimab, dostarlimab, retifanlimab, sasanlimab, penpulimab, CS1003, HLX10, SCT-110A, zimberelimab, balstilimab, genolimzumab, BI 754091, cetrelimab, YBL-006, BAT1306, HX008, budigalimab, AMG 404, CX-188, JTX-4014, 609A, Sym021, LZM009, F520, SG001, AM0001, ENUM 244C8, ENUM 388D4, STI-1110, AK-103, and hAb21. In a specific aspect, a PD-1 binding antagonist is MDX-1106 (nivolumab). In another specific aspect, a PD-1 binding antagonist is MK-3475 (pembrolizumab). In another specific aspect, a PD-1 binding antagonist is a PD-L2 Fc fusion protein, e.g., AMP-224. In another specific aspect, a PD-1 binding antagonist is MED1-0680. In another specific aspect, a PD-1 binding antagonist is PDR001 (spartalizumab). In another specific aspect, a PD-1 binding antagonist is REGN2810 (cemiplimab). In another specific aspect, a PD-1 binding antagonist is BGB-108. In another specific aspect, a PD-1 binding antagonist is prolgolimab. In another specific aspect, a PD-1 binding antagonist is camrelizumab. In another specific aspect, a PD-1 binding antagonist is sintilimab. In another specific aspect, a PD-1 binding antagonist is tislelizumab. In another specific aspect, a PD-1 binding antagonist is toripalimab. Other additonal exemplary PD-1 binding antagonists include BION-004, CB201, AUNP-012, ADG104, and LBL-006.

The term “PD-L2 binding antagonist” refers to a molecule that decreases, blocks, inhibits, abrogates or interferes with signal transduction resulting from the interaction of PD-L2 with either one or more of its binding partners, such as PD-1. PD-L2 (programmed death ligand 2) is also referred to in the art as “programmed cell death 1 ligand 2,” “PDCD1LG2,” “CD273,” “B7-DC,” “Btdc,” and “PDL2.” An exemplary human PD-L2 is shown in UniProtKB/Swiss-Prot Accession No. Q9BQ51. In some instances, a PD-L2 binding antagonist is a molecule that inhibits the binding of PD-L2 to one or more of its binding partners. In a specific aspect, the PD-L2 binding antagonist inhibits binding of PD-L2 to PD-1. Exemplary PD-L2 antagonists include anti-PD-L2 antibodies, antigen binding fragments thereof, immunoadhesins, fusion proteins, oligopeptides and other molecules that decrease, block, inhibit, abrogate or interfere with signal transduction resulting from the interaction of PD-L2 with either one or more of its binding partners, such as PD-1. In one aspect, a PD-L2 binding antagonist reduces the negative co-stimulatory signal mediated by or through cell surface proteins expressed on T lymphocytes mediated signaling through PD-L2 so as render a dysfunctional T-cell less dysfunctional (e.g., enhancing effector responses to antigen recognition). In some aspects, the PD-L2 binding antagonist binds to PD-L2. In some aspects, a PD-L2 binding antagonist is an immunoadhesin. In other aspects, a PD-L2 binding antagonist is an anti-PD-L2 antagonist antibody.

The terms “programmed death ligand 1” and “PD-L1” refer herein to native sequence human PD-L1 polypeptide. Native sequence PD-L1 polypeptides are provided under Uniprot Accession No. Q9NZQ7. For example, the native sequence PD-L1 may have the amino acid sequence as set forth in Uniprot Accession No. Q9NZQ7-1 (isoform 1). In another example, the native sequence PD-L1 may have the amino acid sequence as set forth in Uniprot Accession No. Q9NZQ7-2 (isoform 2). In yet another example, the native sequence PD-L1 may have the amino acid sequence as set forth in Uniprot Accession No. Q9NZQ7-3 (isoform 3). PD-L1 is also referred to in the art as “programmed cell death 1 ligand 1,” “PDCD1LG1,” “CD274,” “B7-H,” and “PDL1.”

The Kabat numbering system is generally used when referring to a residue in the variable domain (approximately residues 1-107 of the light chain and residues 1-113 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG1 EU antibody. For the purposes herein, “atezolizumab” is an Fc-engineered, humanized, non-glycosylated IgG1 kappa immunoglobulin that binds PD-L1 and comprises the heavy chain sequence of SEQ ID NO: 1 and the light chain sequence of SEQ ID NO: 2. Atezolizumab comprises a single amino acid substitution (asparagine to alanine) at position 297 on the heavy chain (N297A) using EU numbering of Fc region amino acid residues, which results in a non-glycosylated antibody that has minimal binding to Fc receptors. Atezolizumab is also described in WHO Drug Information (International Nonproprietary Names for Pharmaceutical Substances), Proposed INN: List 112, Vol. 28, No. 4, published Jan. 16, 2015 (see page 485).

The term “cancer” refers to a disease caused by an uncontrolled division of abnormal cells in a part of the body. In one instance, the cancer is a lung cancer, e.g., NSCLC. The cancer may be locally advanced or metastatic. In some instances, the cancer is locally advanced. In other instances, the cancer is metastatic. In some instances, the cancer may be resected (e.g., completely resected). In other instances, the cancer may be unresectable (e.g., unresectable locally advanced or metastatic cancer). In some instances, the NSCLC is stage IB-IIIA NSCLC, optionally wherein the staging is per the Union Internationale Contre le Cancer/American Joint Committee on Cancer (UICC/AJCC) staging system, 7th edition. In some instances, the NSCLC is stage II-IIIA NSCLC. In some instances, the NSCLC is squamous NSCLC. In other instances, the NSCLC is non-squamous NSCLC.

As used herein, “treating” comprises effective cancer treatment with an effective amount of a therapeutic agent (e.g., a PD-1 axis binding antagonist (e.g., atezolizumab) or combination of therapeutic agents (e.g., a PD-1 axis antagonist and one or more additional therapeutic agents). Treating herein includes, inter alia, adjuvant therapy, neoadjuvant therapy, non-metastatic cancer therapy (e.g., locally advanced cancer therapy), and metastatic cancer therapy. The treatment may be first-line treatment (e.g., the individual may be previously untreated or not have received prior systemic therapy), or second line or later treatment. In some instances, the treating is following an adjuvant therapy (e.g., a platinum-based adjuvant therapy).

Herein, an “effective amount” refers to the amount of a therapeutic agent (e.g., a PD-1 axis binding antagonist (e.g., atezolizumab) or a combination of therapeutic agents (e.g., a PD-1 axis antagonist and one or more additional therapeutic agents)), that achieves a therapeutic result. In some instances, the effective amount of a therapeutic agent or a combination of therapeutic agents is the amount of the agent or of the combination of agents that achieves a clinical benefit, i.e., a clinical endpoint of improved survival (e.g., disease-free survival (DFS)). Improvement (e.g., in terms of survival (e.g., DFS) may be relative to a suitable reference treatment, for example, treatment that does not include the PD-1 axis binding antagonist and/or best supportive care (BSC).

As used herein, “disease-free survival” and “DFS” refer to the length of time after a primary treatment (e.g., surgical resection) that the individual survives without recurrence of the lung cancer (e.g., NSCLC). For example, DFS may be defined as the time from randomization to the date of occurrence of any of the following, whichever occurs first: (i) first recurrence of NSCLC, as determined by the investigator after an integrated assessment of radiographic data, biopsy results (if available), and clinical status; (ii) occurrence of new primary NSCLC, as assessed by the investigator; or (iii) death from any cause.

As used herein, “best supportive care” and “BSC” can refer to care given to improve the quality of life of an individual having a cancer, such as NSCLC. BSC may include palliative care (e.g., monitoring and management of disease-related symptoms) without administration of an anti-cancer therapy (e.g., a PD-1 axis binding antagonist) to the individual. For example, in some instances, best supportive care may include periodic chest X-rays and/or CT scans. In some instances, BSC may include assessments made every three weeks during the first year of treatment, including formal clinic visits and/or clinical contacts (either via telephone call or formal outpatient clinic visit) for symptom and adverse event assessment.

As used herein, the term “chemotherapeutic agent” refers to a compound useful in the treatment of cancer, such as NSCLC. Examples of chemotherapeutic agents include EGFR inhibitors (including small molecule inhibitors (e.g., erlotinib (TARCEVA®, Genentech/OSI Pharm.); PD 183805 (CI 1033, 2-propenamide, N-[4-[(3-chloro-4-fluorophenyl)amino]-7-[3-(4-morpholinyl) propoxy]-6-quinazolinyl]-, dihydrochloride, Pfizer Inc.); ZD1839, gefitinib (IRESSA®) 4-(3′-Chloro-4′-fluoroanilino)-7-methoxy-6-(3-morpholinopropoxy) quinazoline, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(dimethylamino)-2-butenamide) (Wyeth); AG1478 (Pfizer); AG1571 (SU 5271; Pfizer); and dual EGFR/HER2 tyrosine kinase inhibitors such as lapatinib (TYKERBR, GSK572016 or N-[3-chloro-4-[(3 fluorophenyl) methoxy]phenyl]-6[5[[[2methylsulfonyl)ethyl]amino]methyl]-2-furanyl]-4-quinazolinamine)); a tyrosine kinase inhibitor (e.g., an EGFR inhibitor; a small molecule HER2 tyrosine kinase inhibitor such as TAK165 (Takeda); CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; PKI-166 (Novartis); pan-HER inhibitors such as canertinib (CI-1033; Pharmacia); Raf-1 inhibitors such as antisense agent ISIS-5132 (ISIS Pharmaceuticals) which inhibit Raf-1 signaling; non-HER-targeted tyrosine kinase inhibitors such as imatinib mesylate (GLEEVEC®, Glaxo SmithKline); multi-targeted tyrosine kinase inhibitors such as sunitinib (SUTENT®, Pfizer); VEGF receptor tyrosine kinase inhibitors such as vatalanib (PTK787/ZK222584, Novartis/Schering AG); MAPK extracellular regulated kinase I inhibitor CI-1040 (Pharmacia); quinazolines, such as PD 153035,4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d]pyrimidines; curcumin (diferuloyl methane, 4,5-bis(4-fluoroanilino) phthalimide); tyrphostines containing nitrothiophene moieties; PD-0183805 (Warner-Lamber); antisense molecules (e.g., those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); Affinitac (ISIS 3521; Isis/Lilly); PKI 166 (Novartis); GW2016 (Glaxo SmithKline); CI-1033 (Pfizer); EKB-569 (Wyeth); Semaxinib (Pfizer); ZD6474 (AstraZeneca); PTK-787 (Novartis/Schering AG); INC-1C11 (Imclone); and rapamycin (sirolimus, RAPAMUNE®)); proteasome inhibitors such as bortezomib (VELCADE®, Millennium Pharm.); disulfiram; epigallocatechin gallate; salinosporamide A; carfilzomib; 17-AAG (geldanamycin); radicicol; lactate dehydrogenase A (LDH-A); fulvestrant (FASLODEX®, AstraZeneca); letrozole (FEMARA®, Novartis), finasunate (VATALANIBR, Novartis); oxaliplatin (ELOXATIN®, Sanofi); 5-FU (5-fluorouracil); leucovorin; lonafamib (SCH 66336); sorafenib (NEXAVAR®, Bayer Labs); AG1478, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including topotecan and irinotecan); bryostatin; callystatin; CC-1065 (including its adozelesin, carzelesin and bizelesin synthetic analogs); cryptophycins (particularly cryptophycin 1 and cryptophycin 8); adrenocorticosteroids (including prednisone and prednisolone); cyproterone acetate; 5α-reductases including finasteride and dutasteride); vorinostat, romidepsin, panobinostat, valproic acid, mocetinostat dolastatin; aldesleukin, talc duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimustine; antibiotics such as the enediyne antibiotics (e.g., calicheamicin, especially calicheamicin γ1 and calicheamicin ω1); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, cactinomycin, carabicin, caminomycin, carzinophilin, chromomycinis, dactinomycin, detorubicin, 6-diazo-5-oxo-L-norleucine, morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidamnol; nitraerine; pentostatin; phenamet; pirarubicin; losoxantrone; podophyllinic acid; 2-ethylhydrazide; procarbazine; PSK® polysaccharide complex (JHS Natural Products); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; chloranmbucil; GEMZAR® (gemcitabine); 6-thioguanine; mercaptopurine; methotrexate; etoposide (VP-16); ifosfamide; mitoxantrone; novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids, prodrugs, and derivatives of any of the above.

Chemotherapeutic agents also include (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, iodoxyfene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4 (5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide and goserelin; buserelin, tripterelin, medroxyprogesterone acetate, diethylstilbestrol, premarin, fluoxymesterone, all transretionic acid, fenretinide, as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-alpha, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; (ix) growth inhibitory agents including vinca alkaloids (e.g., vincristine, vinblastine, or NAVELBINE® (vinorelbine)), taxanes (e.g., paclitaxel, nab-paclitaxel, and docetaxel), topoisomerase II inhibitors (e.g., doxorubicin, epirubicin, daunorubicin, etoposide, and bleomycin), and DNA alkylating agents (e.g., tamoxigen, prednisone, dacarbazine, mechlorethamine, cisplatin, methotrexate, 5-fluorouracil, and ara-C); and (x) pharmaceutically acceptable salts, acids, prodrugs, and derivatives of any of the above.

Chemotherapeutic agents also include “platinum-based” chemotherapeutic agents, which comprise an organic compound which contains platinum as an integral part of the molecule. Typically, platinum-based chemotherapeutic agents are coordination complexes of platinum. Platinum-based chemotherapeutic agents are sometimes called “platins” in the art. Examples of platinum-based chemotherapeutic agents include, but are not limited to, cisplatin, carboplatin, oxaliplatin, nedaplatin, triplatin tetranitrate, phenanthriplatin, picoplatin, lipoplatin, and satraplatin. Platinum-based chemotherapeutic agents (e.g., cisplatin or carboplatin) may be administered in combination with one or more additional chemotherapeutic agents, e.g., a vinca alkaloid (e.g., vinorelbine), a taxane (e.g., docetaxel), an anti-metabolite (e.g., gemcitabine or pemetrexed), or a combination thereof.

A “platinum-based adjuvant therapy,” as used herein, refers to an adjuvant chemotherapy regimen that includes a platinum-based chemotherapeutic agent. For example, a platinum-based chemotherapy may include a platinum-based chemotherapeutic agent (e.g., cisplatin or carboplatin) in combination with one or more additional chemotherapeutic agents, e.g., a vinca alkaloid (e.g., vinorelbine), a taxane (e.g., docetaxel), an anti-metabolite (e.g., gemcitabine or pemetrexed), or a combination thereof.

The term “cytotoxic agent” as used herein refers to any agent that is detrimental to cells (e.g., causes cell death, inhibits proliferation, or otherwise hinders a cellular function). Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents; enzymes and fragments thereof such as nucleolytic enzymes; and toxins such as small molecule toxins or enzymatically active toxins of bacterial, fungal, plant or animal origin, including fragments and/or variants thereof. Exemplary cytotoxic agents can be selected from anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, inhibitors of LDH-A, inhibitors of fatty acid biosynthesis, cell cycle signaling inhibitors, HDAC inhibitors, proteasome inhibitors, and inhibitors of cancer metabolism. In one instance, the cytotoxic agent is a platinum-based chemotherapeutic agent (e.g., carboplatin or cisplatin). In one instance, the cytotoxic agent is an antagonist of EGFR, e.g., N-(3-ethynylphenyl)-6,7-bis(2-methoxyethoxy) quinazolin-4-amine (e.g., erlotinib). In one instance the cytotoxic agent is a RAF inhibitor, e.g., a BRAF and/or CRAF inhibitor. In one instance the RAF inhibitor is vemurafenib. In one instance, the cytotoxic agent is a PI3K inhibitor.

A “taxane” as used herein is an agent (e.g., a diterpene) which may bind to tubulin, promoting microtubule assembly and stabilization and/or prevent microtubule depolymerization. Exemplary taxanes include, but are not limited to, paclitaxel (i.e., TAXOL®, CAS #33069-62-4), docetaxel (i.e., TAXOTERE®, CAS #114977-28-5), larotaxel, cabazitaxel, milataxel, tesetaxel, and/or orataxel. Taxanes included herein also include taxoid 10-deacetylbaccatin Ill and/or derivatives thereof. In some embodiments, the taxane is an albumin-coated nanoparticle (e.g., nano-albumin bound (nab)-paclitaxel, i.e., ABRAXANE® and/or nab-docetaxel, ABI-008). In some embodiments, the taxane is nab-paclitaxel (ABRAXANE®). In some embodiments, the taxane is formulated in CREMAPHOR® (e.g., TAXOL®) and/or in TWEEN® such as polysorbate 80 (e.g., TAXOTERE®). In some embodiments, the taxane is liposome-encapsulated taxane. In some embodiments, the taxane is a prodrug form and/or conjugated form of taxane (e.g., DHA covalently conjugated to paclitaxel, paclitaxel poliglumex, and/or linoleyl carbonate-paclitaxel). In some embodiments, the paclitaxel is formulated with substantially no surfactant (e.g., in the absence of CREMAPHOR® and/or TWEEN®, such as TOCOSOL® paclitaxel).

The term “vinca alkaloid” refers to a group of anti-mitotic and anti-microtubule alkaloid agents. Vinca alkaloids were originally derived from the periwinkle plant Catharanthus roseus (Vinca rosea) and other Vinca plants. Vinca alkaloids may be naturally occurring (e.g., vinblastine and vincristine) or semi-synthetic (e.g., vinorelbine). Exemplary vinca alkaloids include vinorelbine, vindesine, vinblastine, and vincristine.

The term “individual” refers to a human individual. For example, the individual may be an adult.

The term “antibody” herein specifically covers monoclonal antibodies (including full-length monoclonal antibodies), polyclonal antibodies, multispecific antibodies (e.g., bispecific antibodies), and antibody fragments so long as they exhibit the desired biological activity. In one instance, the antibody is a full-length monoclonal antibody.

The term IgG “isotype” or “subclass” as used herein is meant any of the subclasses of immunoglobulins defined by the chemical and antigenic characteristics of their constant regions.

Depending on the amino acid sequences of the constant domains of their heavy chains, antibodies (immunoglobulins) can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, γ, ε, γ, and μ, respectively. The subunit structures and three-dimensional configurations of different classes of immunoglobulins are well known and described generally in, for example, Abbas et al. Cellular and Mol. Immunology, 4th ed. (W.B. Saunders, Co., 2000). An antibody may be part of a larger fusion molecule, formed by covalent or non-covalent association of the antibody with one or more other proteins or peptides.

The terms “full-length antibody,” “intact antibody,” and “whole antibody” are used herein interchangeably to refer to an antibody in its substantially intact form, not antibody fragments as defined below. The terms refer to an antibody comprising an Fc region.

The term “Fc region” herein is used to define a C-terminal region of an immunoglobulin heavy chain that contains at least a portion of the constant region. The term includes native sequence Fc regions and variant Fc regions. In one aspect, a human IgG heavy chain Fc region extends from Cys226, or from Pro230, to the carboxyl-terminus of the heavy chain. However, antibodies produced by host cells may undergo post-translational cleavage of one or more, particularly one or two, amino acids from the C-terminus of the heavy chain. Therefore, an antibody produced by a host cell by expression of a specific nucleic acid molecule encoding a full-length heavy chain may include the full-length heavy chain, or it may include a cleaved variant of the full-length heavy chain. This may be the case where the final two C-terminal amino acids of the heavy chain are glycine (G446) and lysine (K447). Therefore, the C-terminal lysine (Lys447), or the C-terminal glycine (Gly446) and lysine (Lys447), of the Fc region may or may not be present. Amino acid sequences of heavy chains including an Fc region are denoted herein without the C-terminal lysine (Lys447) if not indicated otherwise. In one aspect, a heavy chain including an Fc region as specified herein, comprised in an antibody disclosed herein, comprises an additional C-terminal glycine-lysine dipeptide (G446 and K447). In one aspect, a heavy chain including an Fc region as specified herein, comprised in an antibody disclosed herein, comprises an additional C-terminal glycine residue (G446). In one aspect, a heavy chain including an Fc region as specified herein, comprised in an antibody disclosed herein, comprises an additional C-terminal lysine residue (K447). In one embodiment, the Fc region contains a single amino acid substitution N297A of the heavy chain. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

A “naked antibody” refers to an antibody that is not conjugated to a heterologous moiety (e.g., a cytotoxic moiety) or radiolabel. The naked antibody may be present in a pharmaceutical composition.

“Antibody fragments” comprise a portion of an intact antibody, preferably comprising the antigen-binding region thereof. In some instances, the antibody fragment described herein is an antigen-binding fragment. Examples of antibody fragments include Fab, Fab′, F(ab′) 2, and Fv fragments; diabodies; linear antibodies; single-chain antibody molecules (e.g., scFvs); and multispecific antibodies formed from antibody fragments.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies in accordance with the present invention may be made by a variety of techniques, including but not limited to the hybridoma method, recombinant DNA methods, phage-display methods, and methods utilizing transgenic animals containing all or part of the human immunoglobulin loci.

The term “hypervariable region” or “HVR” as used herein refers to each of the regions of an antibody variable domain which are hypervariable in sequence and which determine antigen binding specificity, for example “complementarity determining regions” (“CDRs”).

Generally, antibodies comprise six CDRs: three in the VH (CDR-H1, CDR-H2, CDR-H3), and three in the VL (CDR-L1, CDR-L2, CDR-L3). Exemplary CDRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991)); and
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262:732-745 (1996)). Unless otherwise indicated, the CDRs are determined according to Kabat et al., supra. One of skill in the art will understand that the CDR designations can also be determined according to Chothia, supra, McCallum, supra, or any other scientifically accepted nomenclature system.

“Framework” or “FR” refers to variable domain residues other than complementary determining regions (CDRs). The FR of a variable domain generally consists of four FR domains: FR1, FR2, FR3, and FR4. Accordingly, the CDR and FR sequences generally appear in the following sequence in VH (or VL): FR1-CDR-H1 (CDR-L1)-FR2-CDR-H2 (CDR-L2)-FR3-CDR-H3 (CDR-L3)-FR4.

The term “variable domain residue numbering as in Kabat” or “amino acid position numbering as in Kabat,” and variations thereof, refers to the numbering system used for heavy chain variable domains or light chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy chain variable domain may include a single amino acid insert (residue 52a according to Kabat) after residue 52 of H2 and inserted residues (e.g., residues 82a, 82b, and 82c, etc., according to Kabat) after heavy chain FR residue 82. The Kabat numbering of residues may be determined for a given antibody by alignment at regions of homology of the sequence of the antibody with a “standard” Kabat numbered sequence.

The term “package insert” is used to refer to instructions customarily included in commercial packages of therapeutic products, that contain information about the indications, usage, dosage, administration, combination therapy, contraindications and/or warnings concerning the use of such therapeutic products.

As used herein, “in combination with” refers to administration of one treatment modality in addition to another treatment modality, for example, a treatment regimen that includes administration of a PD-1 axis binding antagonist (e.g., atezolizumab) and one or more additional therapeutic agents. As such, “in combination with” refers to administration of one treatment modality before, during, or after administration of the other treatment modality to the individual.

A drug that is administered “concurrently” with one or more other drugs is administered during the same treatment cycle, on the same day of treatment, as the one or more other drugs, and, optionally, at the same time as the one or more other drugs. For instance, for cancer therapies given every 3 weeks, the concurrently administered drugs are each administered on day 1 of a 3-week cycle.

The term “detection” includes any means of detecting, including direct and indirect detection.

The term “biomarker” as used herein refers to an indicator, e.g., predictive, diagnostic, and/or prognostic, which can be detected in a sample, for example, PD-L1. The biomarker may serve as an indicator of a particular subtype of a disease or disorder (e.g., cancer, e.g., NSCLC) characterized by certain, molecular, pathological, histological, and/or clinical features. In some embodiments, a biomarker is a gene. Biomarkers include, but are not limited to, polynucleotides (e.g., DNA and/or RNA), polynucleotide copy number alterations (e.g., DNA copy numbers), polypeptides, polypeptide and polynucleotide modifications (e.g., post-translational modifications), carbohydrates, and/or glycolipid-based molecular markers.

The “amount” or “level” of a biomarker associated with an increased clinical benefit to an individual is a detectable level in a biological sample. These can be measured by methods known to one skilled in the art and also disclosed herein. The expression level or amount of biomarker assessed can be used to determine the response to the treatment.

The terms “level of expression” or “expression level” in general are used interchangeably and generally refer to the amount of a biomarker in a biological sample. “Expression” generally refers to the process by which information (e.g., gene-encoded and/or epigenetic information) is converted into the structures present and operating in the cell. Therefore, as used herein, “expression” may refer to transcription into a polynucleotide, translation into a polypeptide, or even polynucleotide and/or polypeptide modifications (e.g., posttranslational modification of a polypeptide). Fragments of the transcribed polynucleotide, the translated polypeptide, or polynucleotide and/or polypeptide modifications (e.g., posttranslational modification of a polypeptide) shall also be regarded as expressed whether they originate from a transcript generated by alternative splicing or a degraded transcript, or from a post-translational processing of the polypeptide, e.g., by proteolysis. “Expressed genes” include those that are transcribed into a polynucleotide as mRNA and then translated into a polypeptide, and also those that are transcribed into RNA but not translated into a polypeptide (for example, transfer and ribosomal RNAs).

As used herein, “TGFβ CAF gene signature” refers to a set of genes comprising one or more of (e.g., one of, two of, three of, four of, five of, six of, seven of, eight of, nine of, ten of, 11 of, 12 of, 13 of, 14 of, 15 of, 16 of, 17 of, 18 of, 19 of, or 20 of) the following genes: LRRC15, FAP, COL11A1, COL10A1, FNDC1, GREM1, ITGA11, WISP1, PLPP4, PODNL1, TWIST1, COL8A2, SUGCT, NOX4, ZNF469, TENM3, P4HA3, ADAMTS12, and CPZ. In some instances, the TGFβ CAF gene signature comprises the following genes: LRRC15, FAP, COL11A1, COL10A1, FNDC1, GREM1, ITGA11, WISP1, PLPP4, PODNL1, TWIST1, COL8A2, SUGCT, NOX4, ZNF469, TENM3, P4HA3, ADAMTS12, and CPZ. In some instances, the TGFβ CAF gene signature consists of LRRC15, FAP, COL11A1, COL10A1, FNDC1, GREM1, ITGA11, WISP1, PLPP4, PODNL1, TWIST1, COL8A2, SUGCT, NOX4, ZNF469, TENM3, P4HA3, ADAMTS12, and CPZ. In some instances, the expression level of a TGFβ CAF gene signature refers to the mean expression level of the genes comprising the TGFβ CAF gene signature. In some instances, the expression level of a TGFβ CAF gene signature refers to the mean RNA expression level of the genes comprising the TGFβ CAF gene signature. In some instances, the expression level of a TGFβ CAF gene signature refers to the median expression level of the genes comprising the TGFβ CAF gene signature. In some instances, the expression level of a TGFβ CAF gene signature refers to the median RNA expression level of the genes comprising the TGFβ CAF gene signature.

The term “sample,” as used herein, refers to a composition that is obtained or derived from a subject and/or individual of interest that contains a cellular and/or other molecular entity that is to be characterized and/or identified, for example, based on physical, biochemical, chemical, and/or physiological characteristics. For example, the phrase “disease sample” and variations thereof refers to any sample obtained from a subject of interest that would be expected or is known to contain the cellular and/or molecular entity that is to be characterized. Samples include, but are not limited to, tissue samples, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, blood-derived cells, urine, cerebro-spinal fluid, saliva, sputum, tears, perspiration, mucus, tumor lysates, and tissue culture medium, tissue extracts such as homogenized tissue, tumor tissue, cellular extracts, and combinations thereof. In particular embodiments, a sample is a tumor tissue sample. In some instances, in any of the methods, kits, assays, pharmaceutical compositions, articles of manufacture, or uses described herein, a tumor tissue sample is obtained from an individual prior to administration of the anti-PD-L1 antibody. In some instances, in any of the methods, kits, assays, pharmaceutical compositions, articles of manufacture, or uses described herein, a tumor tissue sample is obtained from an individual prior to administration of a platinum-based adjuvant therapy.

By “tissue sample” or “cell sample” is meant a collection of similar cells obtained from a tissue of a subject or individual. The source of the tissue or cell sample may be solid tissue as from a fresh, frozen and/or preserved organ, tissue sample, biopsy, and/or aspirate; blood or any blood constituents such as plasma; bodily fluids such as cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a disease tissue/organ. For instance, a “tumor sample” is a tissue sample obtained from a tumor (e.g., an NSCLC tumor) or other cancerous tissue. The tissue sample may contain a mixed population of cell types (e.g., tumor cells and non-tumor cells, cancerous cells and non-cancerous cells). The tissue sample may contain compounds which are not naturally intermixed with the tissue in nature such as preservatives, anticoagulants, buffers, fixatives, nutrients, antibiotics, or the like.

A “tumor-infiltrating immune cell,” as used herein, refers to any immune cell present in a tumor or a sample thereof. Tumor-infiltrating immune cells include, but are not limited to, intratumoral immune cells, peritumoral immune cells, other tumor stroma cells (e.g., fibroblasts), or any combination thereof. Such tumor-infiltrating immune cells can be, for example, T lymphocytes (such as CD8+T lymphocytes and/or CD4+T lymphocytes), B lymphocytes, or other bone marrow-lineage cells, including granulocytes (e.g., neutrophils, eosinophils, and basophils), monocytes, macrophages, dendritic cells (e.g., interdigitating dendritic cells), histiocytes, and natural killer cells.

A “tumor cell” as used herein, refers to any tumor cell present in a tumor or a sample thereof. Tumor cells may be distinguished from other cells that may be present in a tumor sample, for example, stromal cells and tumor-infiltrating immune cells, using methods known in the art and/or described herein.

A “reference sample,” “reference cell,” “reference tissue,” “control sample,” “control cell,” or “control tissue,” as used herein, refers to a sample, cell, tissue, standard, or level that is used for comparison purposes. In one embodiment, a reference sample, reference cell, reference tissue, control sample, control cell, or control tissue is obtained from a healthy and/or non-diseased part of the body (e.g., tissue or cells) of the same subject or individual. For example, the reference sample, reference cell, reference tissue, control sample, control cell, or control tissue may be healthy and/or non-diseased cells or tissue adjacent to the diseased cells or tissue (e.g., cells or tissue adjacent to a tumor). In another embodiment, a reference sample is obtained from an untreated tissue and/or cell of the body of the same subject or individual. In yet another embodiment, a reference sample, reference cell, reference tissue, control sample, control cell, or control tissue is obtained from a healthy and/or non-diseased part of the body (e.g., tissues or cells) of an individual who is not the subject or individual. In even another embodiment, a reference sample, reference cell, reference tissue, control sample, control cell, or control tissue is obtained from an untreated tissue and/or cell of the body of an individual who is not the subject or individual.

As used herein, a “PD-L1-positive tumor cell fraction” is the percentage of viable tumor cells showing partial or complete membrane staining (exclusive of cytoplasmic staining) at any intensity relative to all viable tumor cells present in a sample, following staining of the sample in the context of an immunohistochemical (IHC) assay, e.g., an IHC assay staining for PD-L1 using the antibody SP142, SP263, 22C3, or 28-8. Accordingly, a PD-L1-positive tumor cell fraction may be calculated using the PD-L1 IHC SP263 (Ventana) assay, for example, by the formula PD-L1-positive tumor cell fraction=(number of PD-L1-positive tumor cells)/(total number of PD-L1-positive and PD-L1 negative tumor cells), wherein PD-L1 cytoplasmic staining of tumor cells and all non-tumor cells (e.g., tumor-infiltrating immune cells, normal cells, necrotic cells, and debris) are excluded from evaluation and scoring. It will be appreciated that any given diagnostic PD-L1 antibody may correspond with a particular IHC assay protocol and/or scoring terminology that can be used to derive a PD-L1-positive tumor cell fraction. For example, a PD-L1-positive tumor cell fraction can be derived from a tumor cell sample stained with SP263, 22C3, SP142, or 28-8 using OPTIVIEW® detection on Benchmark ULTRA, EnVision Flex on AutostainerLink 48, OPTIVIEW® detection and amplification on Benchmark ULTRA, or EnVision Flex on AutostainerLink 48, respectively. In another example, a PD-L1-positive tumor cell fraction may be calculated using the PD-L1 IHC 22C3 pharmDx assay (Dako) according to the formula above. A skilled artisan will appreciate that the sensitivities can vary between different PD-L1 antibodies used in IHC assays. For example, only about 64% of samples that meet a 1% TC or 25% TC threshold, as defined respectively by staining with 28-8 or 22C3 and SP263, meet the threshold when stained using SP142. Hirsch et al., Journal of Thoracic Oncology 12 (2): 208-222, 2016. As used herein, the terms PD-L1-positive tumor cell fraction and “tumor proportion score” (TPS) are used interchangeably.

As used herein, the “Ventana SP142 IHC assay” is conducted according to the Ventana PD-L1 (SP142) Assay package insert (Tucson, AZ: Ventana Medical Systems, Inc.), which is incorporated herein by reference in its entirety. The SP142 antibody is described, e.g., in U.S. Pat. No. 10,689,445, which is incorporated by reference herein in its entirety.

As used herein, the “Ventana SP263 IHC assay” is conducted according to the Ventana PD-L1 (SP263) Assay package insert (Tucson, AZ: Ventana Medical Systems, Inc.), which is incorporated herein by reference in its entirety. The SP263 antibody is described, e.g., in U.S. Patent Application Publication No. US 2015/0346208, which is incorporated by reference herein in its entirety.

As used herein, the “pharmDx 22C3 IHC assay” is conducted according to the PD-L1 IHC 22C3 pharmDx package insert (Carpinteria, CA: Dako, Agilent Pathology Solutions), which is incorporated herein by reference in its entirety.

For the purposes herein a “section” of a tissue sample is meant a single part or piece of a tissue sample, for example, a thin slice of tissue or cells cut from a tissue sample (e.g., a tumor sample). It is to be understood that multiple sections of tissue samples may be taken and subjected to analysis, provided that it is understood that the same section of tissue sample may be analyzed at both morphological and molecular levels, or analyzed with respect to polypeptides (e.g., by immunohistochemistry) and/or polynucleotides (e.g., by in situ hybridization).

### II. Methods and Compositions for Lung Cancer

### A. Therapeutic Methods and Compositions for Treatment of Lung Cancer

Provided herein are methods of treating a lung cancer (e.g., NSCLC) in an individual by determining the expression level of (a) a transforming growth factor-beta (TGFβ) cancer-associated fibroblast (CAF) gene signature in a tumor tissue sample obtained from the individual, wherein an expression level of the TGFβ CAF gene signature has been determined to be at or above a reference TGFβ CAF gene signature expression level, or (b) LRRC15 in a tumor tissue sample obtained from the individual, wherein an expression level of LRRC15 has been determined to be at or above a reference LRRC15 expression level, thereby indicating that the individual is likely to have an increased clinical benefit from an adjuvant treatment comprising a PD-1 axis binding antagonist (e.g., an anti-PD-L1 antibody, e.g., atezolizumab) compared to treatment with best supportive care (BSC) and administering the PD-1 axis binding antagonist. Also provided are PD-1 axis binding antagonists for use in treatment of lung cancer and uses of PD-1 axis binding antagonist for treatment of lung cancer in individuals identified as likely to have an increased clinical benefit from an adjuvant treatment comprising a PD-1 axis binding antagonist (e.g., an anti-PD-L1 antibody, e.g., atezolizumab) compared to treatment with best supportive care (BSC). In some instances, the individual has previously been treated with an adjuvant therapy (e.g., a platinum-based adjuvant therapy, e.g., a cisplatin-based adjuvant therapy).

### (i) Treatment of Individuals Identified by the Transforming Growth Factor-Beta (TGFβ) Cancer-Associated Fibroblast (CAF) Gene Signature

In some aspects, provided herein is a method for treating a lung cancer (e.g., a non-small cell lung cancer (NSCLC)) in an individual, the method comprising: (a) determining the expression level of a transforming growth factor-beta (TGFβ) cancer-associated fibroblast (CAF) gene signature in a tumor tissue sample obtained from the individual, wherein an expression level of the TGFβ CAF gene signature has been determined to be at or above a reference TGFβ CAF gene signature expression level, thereby indicating that the individual is likely to have an increased clinical benefit from an adjuvant treatment comprising the PD-1 axis binding antagonist (e.g., an anti-PD-L1 antibody, e.g., atezolizumab) compared to treatment with best supportive care (BSC); and (b) administering an effective amount of the PD-1 axis binding antagonist (e.g., an anti-PD-L1 antibody, e.g., atezolizumab) to the individual.

In some aspects, provided herein is a method for treating a non-small cell lung cancer (NSCLC) in an individual, the method comprising: (a) determining the expression level of a TGFβ CAF gene signature in a tumor tissue sample obtained from the individual, wherein an expression level of the TGFβ CAF gene signature has been determined to be at or above a reference TGFβ CAF gene signature expression level, thereby indicating that the individual is likely to have an increased clinical benefit from an adjuvant treatment comprising the anti-PD-L1 antibody compared to treatment with best supportive care (BSC); and (b) administering an effective amount of the anti-PD-L1 antibody to the individual, wherein the anti-PD-L1 antibody comprises the following hypervariable regions (HVRs): (i) an HVR-H1 sequence of GFTFSDSWIH (SEQ ID NO: 3); (ii) an HVR-H2 sequence of AWISPYGGSTYYADSVKG (SEQ ID NO: 4); (iii) an HVR-H3 sequence of RHWPGGFDY (SEQ ID NO: 5); (iv) an HVR-L1 sequence of RASQDVSTAVA (SEQ ID NO: 6); (v) an HVR-L2 sequence of SASFLYS (SEQ ID NO: 7); and (vi) an HVR-L3 sequence of QQYLYHPAT (SEQ ID NO: 8).

In some aspects, provided herein is a method of treating an individual having an NSCLC, the method comprising administering to the individual an effective amount of an anti-PD-L1 antibody, wherein a tumor tissue sample from the individual has been determined to have an expression level of a TGFβ CAF gene signature that is at or above a reference TGFβ CAF gene signature expression level, thereby indicating that the individual is likely to have an increased clinical benefit from an adjuvant treatment comprising the anti-PD-L1 antibody compared to treatment with BSC, and wherein the anti-PD-L1 antibody comprises the following HVRs: (i) an HVR-H1 sequence of GFTFSDSWIH (SEQ ID NO: 3); (ii) an HVR-H2 sequence of AWISPYGGSTYYADSVKG (SEQ ID NO: 4); (iii) an HVR-H3 sequence of RHWPGGFDY (SEQ ID NO: 5); (iv) an HVR-L1 sequence of RASQDVSTAVA (SEQ ID NO: 6); (v) an HVR-L2 sequence of SASFLYS (SEQ ID NO: 7); and (vi) an HVR-L3 sequence of QQYLYHPAT (SEQ ID NO: 8).

In some aspects, provided herein is a pharmaceutical composition comprising a PD-1 axis binding antagonist (e.g., an anti-PD-L1 antibody, e.g., atezolizumab) for use in an adjuvant treatment of an individual having a lung cancer (e.g., a non-small cell lung cancer (NSCLC)), wherein the adjuvant treatment comprises administration of an effective amount of the PD-1 axis binding antagonist, wherein a tumor tissue sample obtained from the individual has been determined to have an expression level of an TGFβ CAF gene signature that is at or above a reference TGFβ CAF gene signature expression level, thereby indicating that the individual is likely to have an increased clinical benefit from treatment with the PD-1 axis binding antagonist compared to treatment with BSC. In some embodiments, the PD-1 axis binding antagonist is an anti-PD-L1 antibody, wherein the anti-PD-L1 antibody comprises the following HVRs: (i) an HVR-H1 sequence of GFTFSDSWIH (SEQ ID NO: 3); (ii) an HVR-H2 sequence of AWISPYGGSTYYADSVKG (SEQ ID NO: 4); (iii) an HVR-H3 sequence of RHWPGGFDY (SEQ ID NO: 5); (iv) an HVR-L1 sequence of RASQDVSTAVA (SEQ ID NO: 6); (v) an HVR-L2 sequence of SASFLYS (SEQ ID NO: 7); and (vi) an HVR-L3 sequence of QQYLYHPAT (SEQ ID NO: 8).

In some aspects, provided herein is a use of a PD-1 axis binding antagonist (e.g., an anti-PD-L1 antibody, e.g., atezolizumab) in the manufacture of a medicament for adjuvant treatment of an individual having a lung cancer (e.g., a non-small cell lung cancer (NSCLC)), wherein the adjuvant treatment comprises administration of an effective amount of the PD-1 axis binding antagonist, wherein a tumor tissue sample obtained from the individual has been determined to have an expression level of an TGFβ CAF gene signature that is at or above a reference TGFβ CAF gene signature expression level, thereby indicating that the individual is likely to have an increased clinical benefit from treatment with the PD-1 axis binding antagonist compared to treatment with BSC. In some embodiments, the PD-1 axis binding antagonist is an anti-PD-L1 antibody, wherein the anti-PD-L1 antibody comprises the following HVRs: (i) an HVR-H1 sequence of GFTFSDSWIH (SEQ ID NO: 3); (ii) an HVR-H2 sequence of AWISPYGGSTYYADSVKG (SEQ ID NO: 4); (iii) an HVR-H3 sequence of RHWPGGFDY (SEQ ID NO: 5); (iv) an HVR-L1 sequence of RASQDVSTAVA (SEQ ID NO: 6); (v) an HVR-L2 sequence of SASFLYS (SEQ ID NO: 7); and (vi) an HVR-L3 sequence of QQYLYHPAT (SEQ ID NO: 8).

In some aspects, provided herein is an article of manufacture comprising a PD-1 axis binding antagonist (e.g., an anti-PD-L1 antibody, e.g., atezolizumab) and instructions to administer the PD-1 axis binding antagonist for adjuvant treatment of an individual having a lung cancer (e.g., a non-small cell lung cancer (NSCLC)), wherein the adjuvant treatment comprises administration of an effective amount of the PD-1 axis binding antagonist, wherein a tumor tissue sample obtained from the individual has been determined to have an expression level of an TGFβ CAF gene signature that is at or above a reference TGFβ CAF gene signature expression level, thereby indicating that the individual is likely to have an increased clinical benefit from the adjuvant treatment comprising the PD-1 axis binding antagonist compared to treatment with BSC. In some embodiments, the PD-1 axis binding antagonist is an anti-PD-L1 antibody, wherein the anti-PD-L1 antibody comprises the following HVRs: (i) an HVR-H1 sequence of GFTFSDSWIH (SEQ ID NO: 3); (ii) an HVR-H2 sequence of AWISPYGGSTYYADSVKG (SEQ ID NO: 4); (iii) an HVR-H3 sequence of RHWPGGFDY (SEQ ID NO: 5); (iv) an HVR-L1 sequence of RASQDVSTAVA (SEQ ID NO: 6); (v) an HVR-L2 sequence of SASFLYS (SEQ ID NO: 7); and (vi) an HVR-L3 sequence of QQYLYHPAT (SEQ ID NO: 8).

### (ii) Treatment of Individuals Identified by LRRC15 Gene Expression Level

In some aspects, provided herein is a method for treating a lung cancer (e.g., a non-small cell lung cancer (NSCLC)) in an individual, the method comprising: (a) determining the expression level of LRRC15 in a tumor tissue sample obtained from the individual, wherein an expression level of LRRC15 has been determined to be at or above a reference LRRC15 expression level, thereby indicating that the individual is likely to have an increased clinical benefit from an adjuvant treatment comprising the PD-1 axis binding antagonist (e.g., an anti-PD-L1 antibody, e.g., atezolizumab) compared to treatment with best supportive care (BSC); and (b) administering an effective amount of the PD-1 axis binding antagonist (e.g., an anti-PD-L1 antibody, e.g., atezolizumab) to the individual.

In some aspects, provided herein is a method for treating a non-small cell lung cancer (NSCLC) in an individual, the method comprising: (a) determining the expression level of LRRC15 in a tumor tissue sample obtained from the individual, wherein an expression level of LRRC15 has been determined to be at or above a reference LRRC15 expression level, thereby indicating that the individual is likely to have an increased clinical benefit from an adjuvant treatment comprising the anti-PD-L1 antibody compared to treatment with best supportive care (BSC); and (b) administering an effective amount of the anti-PD-L1 antibody to the individual, wherein the anti-PD-L1 antibody comprises the following hypervariable regions (HVRs): (i) an HVR-H1 sequence of GFTFSDSWIH (SEQ ID NO: 3); (ii) an HVR-H2 sequence of AWISPYGGSTYYADSVKG (SEQ ID NO: 4); (iii) an HVR-H3 sequence of RHWPGGFDY (SEQ ID NO: 5); (iv) an HVR-L1 sequence of RASQDVSTAVA (SEQ ID NO: 6); (v) an HVR-L2 sequence of SASFLYS (SEQ ID NO: 7); and (vi) an HVR-L3 sequence of QQYLYHPAT (SEQ ID NO: 8).

In some aspects, provided herein is a method of treating an individual having an NSCLC, the method comprising administering to the individual an effective amount of an anti-PD-L1 antibody, wherein a tumor tissue sample from the individual has been determined to have an expression level of LRRC15 that is at or above a reference LRRC15 expression level, thereby indicating that the individual is likely to have an increased clinical benefit from an adjuvant treatment comprising the anti-PD-L1 antibody compared to treatment with BSC, and wherein the anti-PD-L1 antibody comprises the following HVRs: (i) an HVR-H1 sequence of GFTFSDSWIH (SEQ ID NO: 3); (ii) an HVR-H2 sequence of AWISPYGGSTYYADSVKG (SEQ ID NO: 4); (iii) an HVR-H3 sequence of RHWPGGFDY (SEQ ID NO: 5); (iv) an HVR-L1 sequence of RASQDVSTAVA (SEQ ID NO: 6); (v) an HVR-L2 sequence of SASFLYS (SEQ ID NO: 7); and (vi) an HVR-L3 sequence of QQYLYHPAT (SEQ ID NO: 8).

In some aspects, provided herein is a pharmaceutical composition comprising a PD-1 axis binding antagonist (e.g., an anti-PD-L1 antibody, e.g., atezolizumab) for use in an adjuvant treatment of an individual having a lung cancer (e.g., a non-small cell lung cancer (NSCLC)), wherein the adjuvant treatment comprises administration of an effective amount of the PD-1 axis binding antagonist, wherein a tumor tissue sample obtained from the individual has been determined to have an expression level of LRRC15 that is at or above a reference LRRC15 expression level, thereby indicating that the individual is likely to have an increased clinical benefit from treatment with the PD-1 axis binding antagonist compared to treatment with BSC. In some embodiments, the PD-1 axis binding antagonist is an anti-PD-L1 antibody, wherein the anti-PD-L1 antibody comprises the following HVRs: (i) an HVR-H1 sequence of GFTFSDSWIH (SEQ ID NO: 3); (ii) an HVR-H2 sequence of AWISPYGGSTYYADSVKG (SEQ ID NO: 4); (iii) an HVR-H3 sequence of RHWPGGFDY (SEQ ID NO: 5); (iv) an HVR-L1 sequence of RASQDVSTAVA (SEQ ID NO: 6); (v) an HVR-L2 sequence of SASFLYS (SEQ ID NO: 7); and (vi) an HVR-L3 sequence of QQYLYHPAT (SEQ ID NO: 8).

In some aspects, provided herein is a use of a PD-1 axis binding antagonist (e.g., an anti-PD-L1 antibody, e.g., atezolizumab) in the manufacture of a medicament for adjuvant treatment of an individual having a lung cancer (e.g., a non-small cell lung cancer (NSCLC)), wherein the adjuvant treatment comprises administration of an effective amount of the PD-1 axis binding antagonist, wherein a tumor tissue sample obtained from the individual has been determined to have an expression level of LRRC15 that is at or above a reference LRRC15 expression level, thereby indicating that the individual is likely to have an increased clinical benefit from treatment with the PD-1 axis binding antagonist compared to treatment with BSC. In some embodiments, the PD-1 axis binding antagonist is an anti-PD-L1 antibody, wherein the anti-PD-L1 antibody comprises the following HVRs: (i) an HVR-H1 sequence of GFTFSDSWIH (SEQ ID NO: 3); (ii) an HVR-H2 sequence of AWISPYGGSTYYADSVKG (SEQ ID NO: 4); (iii) an HVR-H3 sequence of RHWPGGFDY (SEQ ID NO: 5); (iv) an HVR-L1 sequence of RASQDVSTAVA (SEQ ID NO: 6); (v) an HVR-L2 sequence of SASFLYS (SEQ ID NO: 7); and (vi) an HVR-L3 sequence of QQYLYHPAT (SEQ ID NO: 8).

In some aspects, provided herein is an article of manufacture comprising a PD-1 axis binding antagonist (e.g., an anti-PD-L1 antibody, e.g., atezolizumab) and instructions to administer the PD-1 axis binding antagonist for adjuvant treatment of an individual having a lung cancer (e.g., a non-small cell lung cancer (NSCLC)), wherein the adjuvant treatment comprises administration of an effective amount of the PD-1 axis binding antagonist, wherein a tumor tissue sample obtained from the individual has been determined to have an expression level of LRRC15 that is at or above a reference LRRC15 expression level, thereby indicating that the individual is likely to have an increased clinical benefit from the adjuvant treatment comprising the PD-1 axis binding antagonist compared to treatment with BSC. In some embodiments, the PD-1 axis binding antagonist is an anti-PD-L1 antibody, wherein the anti-PD-L1 antibody comprises the following HVRs: (i) an HVR-H1 sequence of GFTFSDSWIH (SEQ ID NO: 3); (ii) an HVR-H2 sequence of AWISPYGGSTYYADSVKG (SEQ ID NO: 4); (iii) an HVR-H3 sequence of RHWPGGFDY (SEQ ID NO: 5); (iv) an HVR-L1 sequence of RASQDVSTAVA (SEQ ID NO: 6); (v) an HVR-L2 sequence of SASFLYS (SEQ ID NO: 7); and (vi) an HVR-L3 sequence of QQYLYHPAT (SEQ ID NO: 8).

### B. Diagnostic Methods and Compositions for Treatment of Lung Cancer

Provided herein are diagnostic methods for identifying an individual having a lung cancer (e.g., a non-small cell lung cancer (NSCLC)) who is likely to have an increased clinical benefit from an adjuvant treatment comprising a PD-1 axis binding antagonist (e.g., an anti-PD-L1 antibody, e.g., atezolizumab) compared to treatment with best supportive care (BSC) and methods for selecting a therapy for an individual having a lung cancer by determining the expression level of (a) a transforming growth factor-beta (TGFβ) cancer-associated fibroblast (CAF) gene signature in a tumor tissue sample obtained from the individual, wherein an expression level of the TGFβ CAF gene signature has been determined to be at or above a reference TGFβ CAF gene signature expression leve,I or (b) LRRC15 in a tumor tissue sample obtained from the individual, wherein an expression level of LRRC15 has been determined to be at or above a reference LRRC15 expression level, thereby indicating that the individual is likely to have an increased clinical benefit from an adjuvant treatment comprising the PD-1 axis binding antagonist as compared to treatment with BSC. Also provided are articles of manufacture, assays, and kits for performing the diagnostic methods, i.e., of identifying the individual and/or selecting a therapy for the individual. In some instances, the individual has previously been treated with an adjuvant therapy (e.g., a platinum-based adjuvant therapy, e.g., a cisplatin-based adjuvant therapy).

### (i) Diagnostic Methods Using the Transforming Growth Factor-Beta (TGFβ) Cancer-Associated Fibroblast (CAF) Gene Signature

In some aspects, provided herein is a method of identifying an individual having a lung cancer (e.g., a non-small cell lung cancer (NSCLC)) who is likely to have an increased clinical benefit from an adjuvant treatment comprising a PD-1 axis binding antagonist (e.g., an anti-PD-L1 antibody, e.g., atezolizumab) compared to treatment with BSC, the method comprising determining the expression level of a TGFβ CAF gene signature in a tumor tissue sample obtained from the individual, wherein an expression level of the TGFβ CAF gene signature has been determined to be at or above a reference TGFβ CAF gene signature expression level, thereby indicating that the individual is likely to have an increased clinical benefit from the adjuvant treatment comprising the PD-1 axis binding antagonist compared to treatment with BSC. In some embodiments, the PD-1 axis binding antagonist is an anti-PD-L1 antibody, wherein the anti-PD-L1 antibody comprises the anti-PD-L1 antibody comprises the following HVRs: (i) an HVR-H1 sequence of GFTFSDSWIH (SEQ ID NO: 3); (ii) an HVR-H2 sequence of AWISPYGGSTYYADSVKG (SEQ ID NO: 4); (iii) an HVR-H3 sequence of RHWPGGFDY (SEQ ID NO: 5); (iv) an HVR-L1 sequence of RASQDVSTAVA (SEQ ID NO: 6); (v) an HVR-L2 sequence of SASFLYS (SEQ ID NO: 7); and (vi) an HVR-L3 sequence of QQYLYHPAT (SEQ ID NO: 8).

In some aspects, provided herein is a method for selecting a therapy for an individual having a lung cancer (e.g., a non-small cell lung cancer (NSCLC)), the method comprising determining the expression level of a TGFβ CAF gene signature in a tumor tissue sample obtained from the individual, wherein an expression level of the TGFβ CAF gene signature has been determined to be at or above a reference TGFβ CAF gene signature expression level, thereby indicating that the individual is likely to have an increased clinical benefit from an adjuvant treatment comprising a PD-1 axis binding antagonist (e.g., an anti-PD-L1 antibody, e.g., atezolizumab) compared to treatment with BSC. In some embodiments, the PD-1 axis binding antagonist is an anti-PD-L1 antibody, wherein the anti-PD-L1 antibody comprises the following HVRs: (i) an HVR-H1 sequence of GFTFSDSWIH (SEQ ID NO: 3); (ii) an HVR-H2 sequence of AWISPYGGSTYYADSVKG (SEQ ID NO: 4); (iii) an HVR-H3 sequence of RHWPGGFDY (SEQ ID NO: 5); (iv) an HVR-L1 sequence of RASQDVSTAVA (SEQ ID NO: 6); (v) an HVR-L2 sequence of SASFLYS (SEQ ID NO: 7); and (vi) an HVR-L3 sequence of QQYLYHPAT (SEQ ID NO: 8).

In some instances, the method further comprises administering to the individual an effective amount of the anti-PD-L1 antibody.

In some aspects, provided herein is a kit for identifying an individual having a lung cancer (e.g., a non-small cell lung cancer (NSCLC)) who is likely to have an increased clinical benefit from an adjuvant treatment comprising a PD-1 axis binding antagonist (e.g., an anti-PD-L1 antibody, e.g., atezolizumab) compared to treatment with BSC, the kit comprising reagents for determining the expression level of a TGFβ CAF gene signature in a tumor tissue sample obtained from the individual, wherein an expression level of a TGFβ CAF gene signature that is at or above a reference TGFβ CAF gene signature expression level identifies that the individual as one who is likely to have an increased clinical benefit from the adjuvant treatment comprising the PD-1 axis binding antagonist compared to treatment with BSC. In some embodiments, the PD-1 axis binding antagonist is an anti-PD-L1 antibody, wherein the anti-PD-L1 antibody comprises the following HVRs: (i) an HVR-H1 sequence of GFTFSDSWIH (SEQ ID NO: 3); (ii) an HVR-H2 sequence of AWISPYGGSTYYADSVKG (SEQ ID NO: 4); (iii) an HVR-H3 sequence of RHWPGGFDY (SEQ ID NO: 5); (iv) an HVR-L1 sequence of RASQDVSTAVA (SEQ ID NO: 6); (v) an HVR-L2 sequence of SASFLYS (SEQ ID NO: 7); and (vi) an HVR-L3 sequence of QQYLYHPAT (SEQ ID NO: 8).

In some aspects, provided herein is an assay for identifying an individual having a lung cancer (e.g., a non-small cell lung cancer (NSCLC)) who is a candidate for an adjuvant treatment comprising a PD-1 axis binding antagonist (e.g., an anti-PD-L1 antibody, e.g., atezolizumab), the assay comprising determining the expression level of a TGFβ CAF gene signature in a tumor tissue sample obtained from the individual, wherein an expression level of the TGFβ CAF gene signature has been determined to be at or above a reference TGFβ CAF gene signature expression level, thereby indicating that the individual is likely to have an increased clinical benefit from the adjuvant treatment comprising the PD-1 axis binding antagonist compared to treatment with BSC. In some embodiments, the PD-1 axis binding antagonist is an anti-PD-L1 antibody, wherein the anti-PD-L1 antibody comprises the following HVRs: (i) an HVR-H1 sequence of GFTFSDSWIH (SEQ ID NO: 3); (ii) an HVR-H2 sequence of AWISPYGGSTYYADSVKG (SEQ ID NO: 4); (iii) an HVR-H3 sequence of RHWPGGFDY (SEQ ID NO: 5); (iv) an HVR-L1 sequence of RASQDVSTAVA (SEQ ID NO: 6); (v) an HVR-L2 sequence of SASFLYS (SEQ ID NO: 7); and (vi) an HVR-L3 sequence of QQYLYHPAT (SEQ ID NO: 8).

### (ii) Diagnostic Methods Using LRRC15 Gene Expression Level

In some aspects, provided herein is a method of identifying an individual having a lung cancer (e.g., a non-small cell lung cancer (NSCLC)) who is likely to have an increased clinical benefit from an adjuvant treatment comprising an anti-PD-L1 antibody compared to treatment with BSC, the method comprising determining the expression level of LRRC15 in a tumor tissue sample obtained from the individual, wherein an expression level of LRRC15 has been determined to be at or above a reference LRRC15 expression level, thereby indicating that the individual is likely to have an increased clinical benefit from the adjuvant treatment comprising the PD-1 axis binding antagonist compared to treatment with BSC. In some embodiments, the PD-1 axis binding antagonist is an anti-PD-L1 antibody, wherein the anti-PD-L1 antibody comprises the following HVRs: (i) an HVR-H1 sequence of GFTFSDSWIH (SEQ ID NO: 3); (ii) an HVR-H2 sequence of AWISPYGGSTYYADSVKG (SEQ ID NO: 4); (iii) an HVR-H3 sequence of RHWPGGFDY (SEQ ID NO: 5); (iv) an HVR-L1 sequence of RASQDVSTAVA (SEQ ID NO: 6); (v) an HVR-L2 sequence of SASFLYS (SEQ ID NO: 7); and (vi) an HVR-L3 sequence of QQYLYHPAT (SEQ ID NO: 8).

In some aspects, provided herein is a method for selecting a therapy for an individual having a lung cancer (e.g., a non-small cell lung cancer (NSCLC)), the method comprising determining the expression level of LRRC15 in a tumor tissue sample obtained from the individual, wherein an expression level of LRRC15 has been determined to be at or above a reference LRRC15 expression level, thereby indicating that the individual is likely to have an increased clinical benefit from an adjuvant treatment comprising the anti-PD-L1 antibody compared to treatment with BSC, wherein the anti-PD-L1 antibody comprises the following HVRs: (i) an HVR-H1 sequence of GFTFSDSWIH (SEQ ID NO: 3); (ii) an HVR-H2 sequence of AWISPYGGSTYYADSVKG (SEQ ID NO: 4); (iii) an HVR-H3 sequence of RHWPGGFDY (SEQ ID NO: 5); (iv) an HVR-L1 sequence of RASQDVSTAVA (SEQ ID NO: 6); (v) an HVR-L2 sequence of SASFLYS (SEQ ID NO: 7); and (vi) an HVR-L3 sequence of QQYLYHPAT (SEQ ID NO: 8).

In some instances, the method further comprises administering to the individual an effective amount of the anti-PD-L1 antibody.

In some aspects, provided herein is a kit for identifying an individual having a lung cancer (e.g., a non-small cell lung cancer (NSCLC)) who is likely to have an increased clinical benefit from an adjuvant treatment comprising an anti-PD-L1 antibody compared to treatment with BSC, the kit comprising reagents for determining the expression level of LRRC15 in a tumor tissue sample obtained from the individual, wherein an expression level of LRRC15 that is at or above a reference LRRC15 expression level identifies that the individual as one who is likely to have an increased clinical benefit from the adjuvant treatment comprising the PD-1 axis binding antagonist compared to treatment with BSC. In some embodiments, the PD-1 axis binding antagonist is an anti-PD-L1 antibody, wherein the anti-PD-L1 antibody comprises the following HVRs: (i) an HVR-H1 sequence of GFTFSDSWIH (SEQ ID NO: 3); (ii) an HVR-H2 sequence of AWISPYGGSTYYADSVKG (SEQ ID NO: 4); (iii) an HVR-H3 sequence of RHWPGGFDY (SEQ ID NO: 5); (iv) an HVR-L1 sequence of RASQDVSTAVA (SEQ ID NO: 6); (v) an HVR-L2 sequence of SASFLYS (SEQ ID NO: 7); and (vi) an HVR-L3 sequence of QQYLYHPAT (SEQ ID NO: 8).

In some aspects, provided herein is an assay for identifying an individual having a lung cancer (e.g., a non-small cell lung cancer (NSCLC)) who is a candidate for an adjuvant treatment comprising an anti-PD-L1 antibody, the assay comprising determining the expression level of LRRC15 in a tumor tissue sample obtained from the individual, wherein an expression level of LRRC15 has been determined to be at or above a reference LRRC15 expression level, thereby indicating that the individual is likely to have an increased clinical benefit from the adjuvant treatment comprising the PD-1 axis binding antagonist compared to treatment with BSC. In some embodiments, the PD-1 axis binding antagonist is an anti-PD-L1 antibody, wherein the anti-PD-L1 antibody comprises the following HVRs: (i) an HVR-H1 sequence of GFTFSDSWIH (SEQ ID NO: 3); (ii) an HVR-H2 sequence of AWISPYGGSTYYADSVKG (SEQ ID NO: 4); (iii) an HVR-H3 sequence of RHWPGGFDY (SEQ ID NO: 5); (iv) an HVR-L1 sequence of RASQDVSTAVA (SEQ ID NO: 6); (v) an HVR-L2 sequence of SASFLYS (SEQ ID NO: 7); and (vi) an HVR-L3 sequence of QQYLYHPAT (SEQ ID NO: 8).

Any suitable PD-1 axis binding antagonist may be used in the methods described herein, e.g., any PD-1 axis binding antagonist described in Section IV below. In some instances, the PD-1 axis binding antagonist is a PD-L1 binding antagonist, a PD-1 binding antagonist, or a PD-L2 binding antagonist.

In some instances, PD-1 axis binding antagonist is a PD-L1 binding antagonist. In some instances, the PD-L1 binding antagonist is an anti-PD-L1 antibody. In some instances, the anti-PD-L1 antibody is atezolizumab, durvalumab, avelumab, or MDX-1105.

In some aspects, the anti-PD-L1 antibody comprises the following HVRs: (i) an HVR-H1 sequence of GFTFSDSWIH (SEQ ID NO: 3); (ii) an HVR-H2 sequence of AWISPYGGSTYYADSVKG (SEQ ID NO: 4); (iii) an HVR-H3 sequence of RHWPGGFDY (SEQ ID NO: 5); (iv) an HVR-L1 sequence of RASQDVSTAVA (SEQ ID NO: 6); (v) an HVR-L2 sequence of SASFLYS (SEQ ID NO: 7); and (vi) an HVR-L3 sequence of QQYLYHPAT (SEQ ID NO: 8).

In some aspects, the anti-PD-L1 antibody comprises: (a) a VH comprising the amino acid sequence:

EVOLVESGGGLVQPGGSLRLSCAASGFTFSDSWIHWVRQAPGKGLEWVAWISPYGGSTYYADSVKGRF TISADTSKNTAYLQMNSLRAEDTAVYYCARRHWPGGFDYWGQGTLVTVSS (SEQ ID NO: 9), and a VL comprising the amino acid sequence:

(SEQ ID NO: 10)
DIQMTQSPSSLSASVGDRVTITCRASQDVSTAVAWYQQKPGKAPKLLIYSA
SFLYSGVPSRFSGSGSGTDFTLTISSLQPEDFATYYCQQYLYHPATFGQGT
KVEIKR.

In some aspects, the anti-PD-L1 antibody comprises: (a) a heavy chain comprising amino acid sequence:

EVQLVESGGGLVQPGGSLRLSCAASGFTFSDSWIHWVRQAPGKGLEWVAWISPYGGSTYYADSVKGRF TISADTSKNTAYLQMNSLRAEDTAVYYCARRHWPGGFDYWGQGTLVTVSSASTKGPSVFPLAPSSKSTS GGTAALGCLVKDYFPEPVTVSWNSGALTSGVHTFPAVLQSSGLYSLSSVVTVPSSSLGTQTYICNVNHKP SNTKVDKKVEPKSCDKTHTCPPCPAPELLGGPSVFLFPPKPKDTLMISRTPEVTCVVVDVSHEDPEVKFN WYVDGVEVHNAKTKPREEQYASTYRVVSVLTVLHQDWLNGKEYKCKVSNKALPAPIEKTISKAKGQPRE PQVYTLPPSREEMTKNQVSLTCLVKGFYPSDIAVEWESNGQPENNYKTTPPVLDSDGSFFLYSKLTVDKS RWQQGNVFSCSVMHEALHNHYTQKSLSLSPG (SEQ ID NO: 1), and (b) a light chain comprising the amino acid sequence:

(SEQ ID NO: 2)
DIQMTQSPSSLSASVGDRVTITCRASQDVSTAVAWYQQKPGKAPKLLIYSA
SFLYSGVPSRFSGSGSGTDFTLTISSLQPEDFATYYCQQYLYHPATFGQGT
KVEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSP
VTKSFNRGEC.

In some aspects, the anti-PD-L1 antibody is atezolizumab.

In some instances, in any of the methods, kits, assays, pharmaceutical compositions, articles of manufacture, or uses described herein, the expression level of the TGFβ CAF gene signature is the median of the expression levels of a set of genes comprising the TGFβ CAF gene signature. In some instances, in any of the methods, kits, assays, pharmaceutical compositions, articles of manufacture, or uses described herein, the set of genes comprising the TGFβ CAF gene signature comprises one or more of (e.g., one of, two of, three of, four of, five of, six of, seven of, eight of, nine of, ten of, 11 of, 12 of, 13 of, 14 of, 15 of, 16 of, 17 of, 18 of, 19 of, or 20 of) the following genes: LRRC15, FAP, COL11A1, COL10A1, FNDC1, GREM1, ITGA11, WISP1, PLPP4, PODNL1, TWIST1, COL8A2, SUGCT, NOX4, ZNF469, TENM3, P4HA3, ADAMTS12, and CPZ. In some instances, in any of the methods, kits, assays, pharmaceutical compositions, articles of manufacture, or uses described herein, the set of genes comprising the TGFβ CAF gene signature comprises the following genes: LRRC15, FAP, COL11A1, COL10A1, FNDC1, GREM1, ITGA11, WISP1, PLPP4, PODNL1, TWIST1, COL8A2, SUGCT, NOX4, ZNF469, TENM3, P4HA3, ADAMTS12, and CPZ. In some instances, in any of the methods, kits, assays, pharmaceutical compositions, articles of manufacture, or uses described herein, the set of genes comprising the TGFβ CAF gene signature consists of LRRC15, FAP, COL11A1, COL10A1, FNDC1, GREM1, ITGA11, WISP1, PLPP4, PODNL1, TWIST1, COL8A2, SUGCT, NOX4, ZNF469, TENM3, P4HA3, ADAMTS12, and CPZ.

Any suitable lung cancer (e.g., NSCLC) may be treated using the approach described herein. In some instances, the NSCLC is stage IB-IIIA NSCLC. In some instances, the staging is per the Union Internationale Contre le Cancer/American Joint Committee on Cancer (UICC/AJCC) staging system, 7th edition. In some instances, the NSCLC is stage II-IIIA NSCLC. In some instances, the NSCLC is stage IB NSCLC. In some instances, the NSCLC is stage II NSCLC (e.g., stage IIA or stage IIB NSCLC). In some instances, the NSCLC is stage IIIA NSCLC. In some instances, the NSCLC is squamous NSCLC. In other instances, the NSCLC is non-squamous NSCLC. In some instances, the NSCLC is not an EGFR/ALK+NSCLC.

In some instances, in any of the methods, kits, assays, pharmaceutical compositions, articles of manufacture, or uses described herein, the reference TGFβ CAF gene signature expression level is the median expression level of the TGFβ CAF gene signature in a reference population. In some instances, in any of the methods, kits, assays, pharmaceutical compositions, articles of manufacture, or uses described herein, the reference TGFβ CAF gene signature expression level is the upper quartile (i.e., 75^{th} percentile) expression level of the TGFβ CAF gene signature in a reference population. In some instances, in any of the methods, kits, assays, pharmaceutical compositions, articles of manufacture, or uses described herein, the reference TGFβ CAF gene signature expression level is the upper tertile (i.e., 66^{th} percentile) expression level of the TGFβ CAF gene signature in a reference population.

In some instances, in any of the methods, kits, assays, pharmaceutical compositions, articles of manufacture, or uses described herein, the reference LRRC15 expression level is the median expression level of LRRC15 in a reference population. In some instances, in any of the methods, kits, assays, pharmaceutical compositions, articles of manufacture, or uses described herein, the reference LRRC15 expression level is the upper quartile (i.e., 75^{th} percentile) expression level of LRRC15 in a reference population. In some instances, in any of the methods, kits, assays, pharmaceutical compositions, articles of manufacture, or uses described herein, the reference LRRC15 expression level is the upper tertile (i.e., 66^{th} percentile) expression level of LRRC15 in a reference population.

In some instances, in any of the methods, kits, assays, pharmaceutical compositions, articles of manufacture, or uses described herein, the expression level (e.g., the expression level of the TGFβ CAF gene signature (e.g., the expression level of any of one or more genes that comprises the TGFβ CAF gene signature; and/or the expression level of LRRC15) is an RNA expression level. In some instances, in any of the methods, kits, assays, pharmaceutical compositions, articles of manufacture, or uses described herein, the RNA expression level is determined using RNA sequencing. In some instances, in any of the methods, kits, assays, pharmaceutical compositions, articles of manufacture, or uses described herein, the RNA sequencing is performed using an Illumina sequencing-by-synthesis platform. In some instances, in any of the methods, kits, assays, pharmaceutical compositions, articles of manufacture, or uses described herein, the Illumina sequencing-by-synthesis platform is a NOVASEQ® 6000. In some instances, in any of the methods, kits, assays, pharmaceutical compositions, articles of manufacture, or uses described herein, the RNA sequencing is performed on a library generated using a RIBO-ZERO® Magnetic Gold Kit.

In some instances, in any of the methods, kits, assays, pharmaceutical compositions, articles of manufacture, or uses described herein, the expression level of LRRC15 is the protein expression level of LRRC15. In some instances, the protein expression level of LRRC15 is quantified using immunohistochemistry. In some instances, the immunohistochemistry for detecting LRRC15 protein expression is performed using a Ventana system.

In some instances, in any of the methods, kits, assays, pharmaceutical compositions, articles of manufacture, or uses described herein, the reference population comprises a set of individuals having the NSCLC. In some instances, each individual in the reference population has stage IB-IIIA NSCLC. In some instances, the staging is per the Union Internationale Contre le Cancer/American Joint Committee on Cancer (UICC/AJCC) staging system, 7^{th} edition. In some instances, each individual in the reference population has stage II-IIIA NSCLC. In some instances, each individual in the reference population has stage IB NSCLC. In some instances, each individual in the reference population has stage II NSCLC (e.g., stage IIA or stage IIB NSCLC). In some instances, each individual in the reference population has stage IIIA NSCLC. In some instances, each individual in the reference population has squamous NSCLC. In some instances, each individual in the reference population has non-squamous NSCLC. In some instances, each individual in the reference population has an NSCLC that is not an EGFR/ALK+NSCLC.

In some instances, in any of the methods, kits, assays, pharmaceutical compositions, articles of manufacture, or uses described herein, the tumor tissue sample is obtained from the individual prior to administration of the anti-PD-L1 antibody. In some instances, in any of the methods, kits, assays, pharmaceutical compositions, articles of manufacture, or uses described herein, the tumor tissue sample is obtained from the individual prior to administration of a platinum-based adjuvant therapy.

In some aspects, the increased clinical benefit is an increase in disease-free survival (DFS).

In some instances, the treatment regimen extends the individual's DFS by at least about 1 month, about 2 months, about 3 months, about 4 months, about 5 months, about 6 months, about 7 months, about 8 months, about 9 months, about 10 months, about 11 months, about 12 months, about 13 months, about 14 months, about 15 months, about 16 months, about 17 months, about 14 months, about 15 months, about 16 months, about 17 months, about 18 months, about 19 months, about 20 months, about 21 months, about 22 months, about 23 months, about 24 months, about 3 years, about 4 years, about 5 years, about 6 years, about 7 years, about 8 years, about 9 years, about 10 years, or longer, as compared to BSC.

In some instances, the treatment regimen extends the individual's DFS by about 1 month to about 5 years, about 1 month to about 4 years, about 1 month to about 3 years, about 1 month to about 2 years, about 1 month to about 12 months, about 1 month to about 11 months, about 1 month to about 10 months, about 1 month to about 9 months, about 1 month to about 8 months, about 1 month to about 7 months, about 1 month to about 6 months, about 1 month to about 5 months, about 1 month to about 4 months, about 1 month to about 3 months, about 1 month to about 2 months, about 2 months to about 5 years, about 2 months to about 4 years, about 2 months to about 3 years, about 2 months to about 2 years, about 2 months to about 12 months, about 2 months to about 11 months, about 2 months to about 10 months, about 2 months to about 9 months, about 2 months to about 8 months, about 2 months to about 7 months, about 2 months to about 6 months, about 2 months to about 5 months, about 2 months to about 4 months, about 2 months to about 3 months, about 3 months to about 5 years, about 3 months to about 4 years, about 3 months to about 3 years, about 3 months to about 2 years, about 3 months to about 12 months, about 3 months to about 11 months, about 3 months to about 10 months, about 3 months to about 9 months, about 3 months to about 8 months, about 3 months to about 7 months, about 3 months to about 6 months, about 3 months to about 5 months, about 3 months to about 4 months, about 4 months to about 5 years, about 4 months to about 4 years, about 4 months to about 3 years, about 4 months to about 2 years, about 4 months to about 12 months, about 4 months to about 11 months, about 4 months to about 10 months, about 4 months to about 9 months, about 4 months to about 8 months, about 4 months to about 7 months, about 4 months to about 6 months, about 4 months to about 5 months, about 5 months to about 5 years, about 5 months to about 4 years, about 5 months to about 3 years, about 5 months to about 2 years, about 5 months to about 12 months, about 5 months to about 11 months, about 5 months to about 10 months, about 5 months to about 9 months, about 5 months to about 8 months, about 5 months to about 7 months, about 5 months to about 6 months, about 6 months to about 5 years, about 6 months to about 4 years, about 6 months to about 3 years, about 6 months to about 2 years, about 6 months to about 12 months, about 6 months to about 11 months, about 6 months to about 10 months, about 6 months to about 9 months, about 6 months to about 8 months, about 6 months to about 7 months, about 7 months to about 5 years, about 7 months to about 4 years, about 7 months to about 3 years, about 7 months to about 2 years, about 7 months to about 12 months, about 7 months to about 11 months, about 7 months to about 10 months, about 7 months to about 9 months, about 7 months to about 8 months, about 8 months to about 5 years, about 8 months to about 4 years, about 8 months to about 3 years, about 8 months to about 2 years, about 8 months to about 12 months, about 8 months to about 11 months, about 8 months to about 10 months, about 8 months to about 9 months, about 9 months to about 5 years, about 9 months to about 4 years, about 9 months to about 3 years, about 9 months to about 2 years, about 9 months to about 12 months, about 9 months to about 11 months, about 9 months to about 10 months, about 10 months to about 5 years, about 10 months to about 4 years, about 10 months to about 3 years, about 10 months to about 2 years, about 10 months to about 12 months, about 10 months to about 11 months, about 11 months to about 5 years, about 11 months to about 4 years, about 11 months to about 3 years, about 11 months to about 2 years, about 11 months to about 12 months, about 1 year to about 5 years, about 1 year to about 4 years, about 1 year to about 3 years, about 1 year to about 2 years, about 2 years to about 5 years, about 2 years to about 4 years, about 2 years to about 3 years, about 3 years to about 5 years, about 3 years to about 4 years, or about 4 years to about 5 years.

In some instances, the treatment regimen extends the individual's DFS by about 7 months as compared to BSC.

In some instances, the treatment regimen increases the individual's likelihood of having DFS at 3 years and/or at 5 years after treatment as compared to BSC. In some instances, the treatment regimen increases the individual's likelihood of having DFS at 3 years after treatment as compared to BSC. In some instances, the treatment regimen increases the individual's likelihood of having DFS at 5 years after treatment as compared to BSC. In some instances, the treatment regimen increases the individual's likelihood of having DFS at both 3 years and 5 years after treatment as compared to BSC.

In some aspects, the increased clinical benefit is an increase in overall survival (OS).

In some instances, the treatment regimen extends the individual's OS by at least about 1 month, about 2 months, about 3 months, about 4 months, about 5 months, about 6 months, about 7 months, about 8 months, about 9 months, about 10 months, about 11 months, about 12 months, about 13 months, about 14 months, about 15 months, about 16 months, about 17 months, about 14 months, about 15 months, about 16 months, about 17 months, about 18 months, about 19 months, about 20 months, about 21 months, about 22 months, about 23 months, about 24 months, about 3 years, about 4 years, about 5 years, about 6 years, about 7 years, about 8 years, about 9 years, about 10 years, or longer, as compared to BSC.

In some instances, the treatment regimen extends the individual's OS by about 1 month to about 5 years, about 1 month to about 4 years, about 1 month to about 3 years, about 1 month to about 2 years, about 1 month to about 12 months, about 1 month to about 11 months, about 1 month to about 10 months, about 1 month to about 9 months, about 1 month to about 8 months, about 1 month to about 7 months, about 1 month to about 6 months, about 1 month to about 5 months, about 1 month to about 4 months, about 1 month to about 3 months, about 1 month to about 2 months, about 2 months to about 5 years, about 2 months to about 4 years, about 2 months to about 3 years, about 2 months to about 2 years, about 2 months to about 12 months, about 2 months to about 11 months, about 2 months to about 10 months, about 2 months to about 9 months, about 2 months to about 8 months, about 2 months to about 7 months, about 2 months to about 6 months, about 2 months to about 5 months, about 2 months to about 4 months, about 2 months to about 3 months, about 3 months to about 5 years, about 3 months to about 4 years, about 3 months to about 3 years, about 3 months to about 2 years, about 3 months to about 12 months, about 3 months to about 11 months, about 3 months to about 10 months, about 3 months to about 9 months, about 3 months to about 8 months, about 3 months to about 7 months, about 3 months to about 6 months, about 3 months to about 5 months, about 3 months to about 4 months, about 4 months to about 5 years, about 4 months to about 4 years, about 4 months to about 3 years, about 4 months to about 2 years, about 4 months to about 12 months, about 4 months to about 11 months, about 4 months to about 10 months, about 4 months to about 9 months, about 4 months to about 8 months, about 4 months to about 7 months, about 4 months to about 6 months, about 4 months to about 5 months, about 5 months to about 5 years, about 5 months to about 4 years, about 5 months to about 3 years, about 5 months to about 2 years, about 5 months to about 12 months, about 5 months to about 11 months, about 5 months to about 10 months, about 5 months to about 9 months, about 5 months to about 8 months, about 5 months to about 7 months, about 5 months to about 6 months, about 6 months to about 5 years, about 6 months to about 4 years, about 6 months to about 3 years, about 6 months to about 2 years, about 6 months to about 12 months, about 6 months to about 11 months, about 6 months to about 10 months, about 6 months to about 9 months, about 6 months to about 8 months, about 6 months to about 7 months, about 7 months to about 5 years, about 7 months to about 4 years, about 7 months to about 3 years, about 7 months to about 2 years, about 7 months to about 12 months, about 7 months to about 11 months, about 7 months to about 10 months, about 7 months to about 9 months, about 7 months to about 8 months, about 8 months to about 5 years, about 8 months to about 4 years, about 8 months to about 3 years, about 8 months to about 2 years, about 8 months to about 12 months, about 8 months to about 11 months, about 8 months to about 10 months, about 8 months to about 9 months, about 9 months to about 5 years, about 9 months to about 4 years, about 9 months to about 3 years, about 9 months to about 2 years, about 9 months to about 12 months, about 9 months to about 11 months, about 9 months to about 10 months, about 10 months to about 5 years, about 10 months to about 4 years, about 10 months to about 3 years, about 10 months to about 2 years, about 10 months to about 12 months, about 10 months to about 11 months, about 11 months to about 5 years, about 11 months to about 4 years, about 11 months to about 3 years, about 11 months to about 2 years, about 11 months to about 12 months, about 1 year to about 5 years, about 1 year to about 4 years, about 1 year to about 3 years, about 1 year to about 2 years, about 2 years to about 5 years, about 2 years to about 4 years, about 2 years to about 3 years, about 3 years to about 5 years, about 3 years to about 4 years, or about 4 years to about 5 years.

In some instances, the treatment regimen increases the individual's likelihood of having OS at 3 years and/or at 5 years after treatment as compared to BSC. In some instances, the treatment regimen increases the individual's likelihood of having OS at 3 years after treatment as compared to BSC. In some instances, the treatment regimen increases the individual's likelihood of having OS at 5 years after treatment as compared to BSC. In some instances, the treatment regimen increases the individual's likelihood of having OS at both 3 years and 5 years after treatment as compared to BSC.

Any suitable adjuvant therapy may have been used prior to the treatment with a PD-1 axis binding antagonist, including any adjuvant therapy described in Section V below. In some instances, the adjuvant therapy comprises a platinum-based adjuvant therapy. In some instances, the platinum-based adjuvant therapy comprises cisplatin or carboplatin. In some instances, the adjuvant therapy is an earlier or initial adjuvant therapy used prior to adjuvant treatment with a PD-1 axis binding antagonist (e.g., atezolizumab)

In some instances, the platinum-based adjuvant therapy comprises cisplatin. Cisplatin may be administered at any suitable dose. In some instances, cisplatin was administered at a dose of about 75 mg/m² intravenously on Day 1 of each 21-day cycle.

In some instances, the platinum-based adjuvant therapy further comprises one or more additional chemotherapeutic agents. In some instances, the one or more additional chemotherapeutic agents comprises a vinca alkaloid, a taxane, an anti-metabolite, or a combination thereof. In some instances, the vinca alkaloid is vinorelbine. In some instances, the taxane is docetaxel. In some instances, the anti-metabolite is gemcitabine or pemetrexed. In some instances, the anti-metabolite is gemcitabine. In some instances, the anti-metabolite is pemetrexed.

In some instances, the platinum-based adjuvant therapy comprises up to eight 21-day cycles. For example, the platinum-based adjuvant therapy may comprise one, two, three, four, five, six, seven, or eight 21-day cycles. For example, the platinum-based adjuvant therapy may comprise 1 to 8, 2 to 8, 3 to 8, 4 to 8, 5 to 8, 6 to 8, 7 to 8, 1 to 7, 2 to 7, 3 to 7, 4 to 7, 5 to 7, 6 to 7, 1 to 6, 2 to 6, 3 to 6, 4 to 6, 5 to 6, 1 to 5, 2 to 5, 3 to 5, 4 to 5, 1 to 4, 2 to 4, 3 to 4, 1 to 3, 2 to 3, or 1 to 50 21-day cycles. In some instances, the platinum-based adjuvant therapy comprises up to 4 21-day cycles.

In some instances, the platinum-based adjuvant therapy comprises cisplatin and a vinca alkaloid, e.g., vinorelbine. In some instances, cisplatin was administered at a dose of about 75 mg/m² intravenously on Day 1 of each 21-day cycle, and vinorelbine was administered at a dose of about 30 mg/m² by intravenous push on Days 1 and 8 of each 21-day cycle.

In some instances, the platinum-based adjuvant therapy comprises cisplatin and a taxane, e.g., docetaxel. In some instances, cisplatin was administered at a dose of about 75 mg/m² intravenously on Day 1 of each 21-day cycle, and docetaxel was administered at a dose of about 75 mg/m² intravenously on Day 1 of each 21-day cycle.

In some instances, the platinum-based adjuvant therapy comprises cisplatin and an anti-metabolite, e.g., a nucleoside analogue such as gemcitabine. In some instances, cisplatin was administered at a dose of about 75 mg/m² intravenously on Day 1 of each 21-day cycle, and gemcitabine was administered at a dose of about 1250 mg/m² intravenously on Days 1 and 8 of each 21-day cycle.

In some instances, the platinum-based adjuvant therapy comprises cisplatin and an anti-metabolite, e.g., a folate anti-metabolite such as pemetrexed. In some instances, cisplatin was administered at a dose of about 75 mg/m² intravenously on Day 1 of each 21-day cycle, and pemetrexed was administered at a dose of about 500 mg/m² intravenously on Day 1 of each 21-day cycle.

In some instances, the individual had a complete resection of the NSCLC prior to the adjuvant therapy. In some instances, the complete resection comprises lobectomy, sleeve lobectomy, bilobectomy, or pneumonectomy. In some instances, the individual had complete resection of the NSCLC about 4 to about 12 weeks prior to the adjuvant therapy.

In some instances, the individual has not had treatment with systemic chemotherapy prior to the adjuvant therapy. In other instances, the individual may have had treatment with systemic chemotherapy prior to the adjuvant therapy.

In some instances, the individual is an adult individual.

In some instances, the PD-1 axis binding antagonist is administered in combination with an effective amount of one or more additional therapeutic agents.

In other instances, the PD-1 axis binding antagonist is administered as a monotherapy.

In some instances, atezolizumab is administered as a single-agent for adjuvant treatment following resection and platinum-based chemotherapy for individuals with NSCLC, wherein a tumor tissue sample obtained from the individual has been determined to have an expression level of an TGFβ CAF gene signature that is at or above a reference TGFβ CAF gene signature expression level. In some instances, atezolizumab is administered as a single-agent for adjuvant treatment following resection and platinum-based chemotherapy for individuals with NSCLC, wherein a tumor tissue sample from the individual has been determined to have an expression level of LRRC15 that is at or above a reference LRRC15 expression level.

For example, provided herein is a method of treating NSCLC (e.g., completely resected stage IB-IIIA NSCLC (e.g., stage II-IIIA NSCLC)) in an individual who has previously been treated with a platinum-based adjuvant therapy (e.g., cisplatin-based adjuvant therapy), the method comprising administering to the individual a treatment regimen comprising an effective amount of atezolizumab, wherein the atezolizumab is administered as a monotherapy, wherein a tumor tissue sample obtained from the individual has been determined to have an expression level of an TGFβ CAF gene signature that is at or above a reference TGFβ CAF gene signature expression level. For example, provided herein is a method of treating NSCLC (e.g., completely resected stage IB-IIIA NSCLC (e.g., stage II-IIIA NSCLC)) in an individual who has previously been treated with a platinum-based adjuvant therapy (e.g., cisplatin-based adjuvant therapy), the method comprising administering to the individual a treatment regimen comprising an effective amount of atezolizumab, wherein the atezolizumab is administered as a monotherapy, wherein a tumor tissue sample from the individual has been determined to have an expression level of LRRC15 that is at or above a reference LRRC15 expression level. In some instances, the NSCLC is completely resected stage IB-IIIA NSCLC. In some instances, the NSCLC is stage II-IIIA NSCLC. In some example, the platinum-based adjuvant therapy is cisplatin-based adjuvant therapy. In some instances, the treatment regimen extends the individual's DFS as compared to best supportive care. In some instances, the treatment regimen extends the individual's OS as compared to best supportive care. In some instances, atezolizumab is administered to the individual intravenously at a dose of about 1200 mg on Day 1 of each 21-day dosing cycle. In some instances, atezolizumab is administered to the individual subcutaneously at a dose of about 1875 mg on Day 1 of each 21-day dosing cycle.

In another example, provided herein is atezolizumab for use in treatment of NSCLC (e.g., completely resected stage IB-IIIA NSCLC (e.g., stage II-IIIA NSCLC)) in an individual who has previously been treated with a platinum-based adjuvant therapy (e.g., cisplatin-based adjuvant therapy), the treatment comprising administration of a treatment regimen comprising an effective amount of atezolizumab, wherein the atezolizumab is administered as a monotherapy, wherein a tumor tissue sample obtained from the individual has been determined to have an expression level of an TGFβ CAF gene signature that is at or above a reference TGFβ CAF gene signature expression level. In another example, provided herein is atezolizumab for use in treatment of NSCLC (e.g., completely resected stage IB-IIIA NSCLC (e.g., stage II-IIIA NSCLC)) in an individual who has previously been treated with a platinum-based adjuvant therapy (e.g., cisplatin-based adjuvant therapy), the treatment comprising administration of a treatment regimen comprising an effective amount of atezolizumab, wherein the atezolizumab is administered as a monotherapy, wherein a tumor tissue sample from the individual has been determined to have an expression level of LRRC15 that is at or above a reference LRRC15 expression level. In some instances, the NSCLC is completely resected stage IB-IIIA NSCLC. In some instances, the NSCLC is stage II-IIIA NSCLC. In some instances, the platinum-based adjuvant therapy is cisplatin-based adjuvant therapy. In some instances, the treatment regimen extends the individual's DFS as compared to best supportive care. In some instances, the treatment regimen extends the individual's OS as compared to best supportive care. In some instances, atezolizumab is administered to the individual intravenously at a dose of about 1200 mg on Day 1 of each 21-day dosing cycle. In some instances, atezolizumab is administered to the individual subcutaneously at a dose of about 1875 mg on Day 1 of each 21-day dosing cycle.

In another example, provided herein is a method of adjuvant treatment following resection and platinum-based chemotherapy for an adult individual with stage IB to IIIA NSCLC (e.g., stage II to IIIA NSCLC), the method comprising administering to the individual a treatment regimen comprising an effective amount of atezolizumab, wherein the treatment regimen extends the individual's DFS and/or OS as compared to best supportive care, and wherein a tumor tissue sample obtained from the individual has been determined to have an expression level of an TGFβ CAF gene signature that is at or above a reference TGFβ CAF gene signature expression level. In another example, provided herein is a method of adjuvant treatment following resection and platinum-based chemotherapy for an adult individual with stage IB to IIIA NSCLC (e.g., stage II to IIIA NSCLC), the method comprising administering to the individual a treatment regimen comprising an effective amount of atezolizumab, wherein the treatment regimen extends the individual's DFS and/or OS as compared to best supportive care, and wherein a tumor tissue sample from the individual has been determined to have an expression level of LRRC15 that is at or above a reference LRRC15 expression level.

In another example, provided herein is atezolizumab for use in adjuvant treatment following resection and platinum-based chemotherapy for an adult individual with stage IB-IIIA NSCLC (e.g., stage IB, stage IIA, stage IIB, or stage IIIA NSCLC), wherein the treatment comprises administration of an effective amount of a treatment regimen comprising atezolizumab, wherein the treatment regimen extends the individual's DFS and/or OS as compared to best supportive care, and wherein a tumor tissue sample obtained from the individual has been determined to have an expression level of an TGFβ CAF gene signature that is at or above a reference TGFβ CAF gene signature expression level. In another example, provided herein is atezolizumab for use in adjuvant treatment following resection and platinum-based chemotherapy for an adult individual with stage IB-IIIA NSCLC (e.g., stage IB, stage IIA, stage IIB, or stage IIIA NSCLC), wherein the treatment comprises administration of an effective amount of a treatment regimen comprising atezolizumab, wherein the treatment regimen extends the individual's DFS and/or OS as compared to best supportive care, and wherein a tumor tissue sample from the individual has been determined to have an expression level of LRRC15 that is at or above a reference LRRC15 expression level.

In any of the preceding instances, atezolizumab is administered to the individual as a monotherapy.

In any of the preceding instances, the individual may have had no progression after platinum-based chemotherapy.

In any of the preceding instances, the individual has had up to 4 cycles (e.g., 1, 2, 3, or 4 cycles) of platinum-based chemotherapy.

In any of the preceding instances, atezolizumab may be administered to the individual intravenously at a dose of 8 about 40 mg every 2 weeks, about 1200 mg every 3 weeks, or about 1680 mg every 4 weeks.

In any of the preceding instances, atezolizumab may be administered to the individual intravenously at a dose of about 840 mg every 2 weeks.

In any of the preceding instances, atezolizumab may be administered to the individual intravenously at a dose of about 1200 mg every 3 weeks.

In any of the preceding instances, atezolizumab may be administered to the individual intravenously at a dose of about 1680 mg every 4 weeks.

In any of the preceding instances, atezolizumab may be administered to the individual subcutaneously at a dose of about 1875 mg every 3 weeks.

In any of the preceding instances, atezolizumab is administered to the individual for up to 1 year.

In any of the preceding instances, each dosing cycle may have any suitable length, e.g., about 7 days, about 14 days, about 21 days, about 28 days, or longer. In some instances, each dosing cycle is about 21 days. In some embodiments, each dosing cycle is about 21 days.

The individual is preferably a human.

As a general proposition, the therapeutically effective amount of a PD-1 axis binding antagonist (e.g., atezolizumab) administered to a human will be in the range of about 0.01 to about 50 mg/kg of individual body weight, whether by one or more administrations.

In some exemplary embodiments, the PD-1 axis binding antagonist is administered in a dose of about 0.01 to about 45 mg/kg, about 0.01 to about 40 mg/kg, about 0.01 to about 35 mg/kg, about 0.01 to about 30 mg/kg, about 0.01 to about 25 mg/kg, about 0.01 to about 20 mg/kg, about 0.01 to about 15 mg/kg, about 0.01 to about 10 mg/kg, about 0.01 to about 5 mg/kg, or about 0.01 to about 1 mg/kg administered daily, weekly, every two weeks, every three weeks, or every four weeks, for example.

In one instance, a PD-1 axis binding antagonist is administered to a human at a dose of about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, or about 1500 mg. In some instances, the PD-1 axis binding antagonist may be administered at a dose of about 1000 mg to about 1400 mg every three weeks (e.g., about 1100 mg to about 1300 mg every three weeks, e.g., about 1150 mg to about 1250 mg every three weeks).

In one instance, a PD-1 axis binding antagonist is administered to a human at a dose of about 1500 mg, about 1600 mg, about 1700 mg, about 1800 mg, about 1850 mg, about 1875 mg, about 1900 mg, about 2000 mg, about 2100 mg, or about 2200 mg. In one instances, a PD-1 axis binding antagonist is administered to a human at a dose of about 1500 mg to about 2200 mg every three weeks (e.g., about 1700 mg to about 1900 mg every three weeks; e.g., about 1850 mg to about 1900 mg every three weeks; e.g., about 1875 mg every three weeks).

In some instances, an individual is administered a total of 1 to 50 doses of a PD-1 axis binding antagonist, e.g., 1 to 50 doses, 1 to 45 doses, 1 to 40 doses, 1 to 35 doses, 1 to 30 doses, 1 to 25 doses, 1 to 20 doses, 1 to 15 doses, 1 to 10 doses, 1 to 5 doses, 2 to 50 doses, 2 to 45 doses, 2 to 40 doses, 2 to 35 doses, 2 to 30 doses, 2 to 25 doses, 2 to 20 doses, 2 to 15 doses, 2 to 10 doses, 2 to 5 doses, 3 to 50 doses, 3 to 45 doses, 3 to 40 doses, 3 to 35 doses, 3 to 30 doses, 3 to 25 doses, 3 to 20 doses, 3 to 15 doses, 3 to 10 doses, 3 to 5 doses, 4 to 50 doses, 4 to 45 doses, 4 to 40 doses, 4 to 35 doses, 4 to 30 doses, 4 to 25 doses, 4 to 20 doses, 4 to 15 doses, 4 to 10 doses, 4 to 5 doses, 5 to 50 doses, 5 to 45 doses, 5 to 40 doses, 5 to 35 doses, 5 to 30 doses, 5 to 25 doses, 5 to 20 doses, 5 to 15 doses, 5 to 10 doses, 10 to 50 doses, 10 to 45 doses, 10 to 40 doses, 10 to 35 doses, 10 to 30 doses, 10 to 25 doses, 10 to 20 doses, 10 to 15 doses, 15 to 50 doses, 15 to 45 doses, 15 to 40 doses, 15 to 35 doses, 15 to 30 doses, 15 to 25 doses, 15 to 20 doses, 20 to 50 doses, 20 to 45 doses, 20 to 40 doses, 20 to 35 doses, 20 to 30 doses, 20 to 25 doses, 25 to 50 doses, 25 to 45 doses, 25 to 40 doses, 25 to 35 doses, 25 to 30 doses, 30 to 50 doses, 30 to 45 doses, 30 to 40 doses, 30 to 35 doses, 35 to 50 doses, 35 to 45 doses, 35 to 40 doses, 40 to 50 doses, 40 to 45 doses, or 45 to 50 doses. In particular instances, the doses may be administered intravenously. In particular instances, the doses may be administered subcutaneously. In some instances, an individual is administered up to 16 doses (e.g., 1, 2, 3, 4, 5, 6, 7, 8, 9, 10, 11, 12, 13, 14, 15, or 16 doses) of a PD-1 axis binding antagonist. In some instances, an individual is administered 16 doses of a PD-1 axis binding antagonist.

In some instances, atezolizumab is administered to the individual intravenously at a dose of about 840 mg every 2 weeks, about 1200 mg every 3 weeks, or about 1680 mg of every 4 weeks. In some instances, atezolizumab is administered to the individual intravenously at a dose of 1200 mg every three weeks. In some instances, atezolizumab is administered to the individual intravenously at a dose of 1200 mg on Day 1 of each 21-day dosing cycle. In some instances, atezolizumab is administered to the individual subcutaneously at a dose of 1875 mg every three weeks. In some instances, atezolizumab is administered to the individual subcutaneously at a dose of 1875 mg on Day 1 of each 21-day dosing cycle.

The PD-1 axis binding antagonist and/or any additional therapeutic agent(s) may be administered in any suitable manner known in the art. For example, the PD-1 axis binding antagonist and/or any additional therapeutic agent(s) may be administered sequentially (on different days) or concurrently (on the same day or during the same treatment cycle). In some instances, the PD-1 axis binding antagonist is administered prior to the additional therapeutic agent. In other instances, the PD-1 axis binding antagonist is administered after the additional therapeutic agent. In some instances, the PD-1 axis binding antagonist and/or any additional therapeutic agent(s) may be administered on the same day. In some instances, the PD-1 axis binding antagonist may be administered prior to an additional therapeutic agent that is administered on the same day. For example, the PD-1 axis binding antagonist may be administered prior to chemotherapy on the same day. In another example, the PD-1 axis binding antagonist may be administered prior to both chemotherapy and another drug (e.g., bevacizumab) on the same day. In other instances, the PD-1 axis binding antagonist may be administered after an additional therapeutic agent that is administered on the same day. In yet other instances, the PD-1 axis binding antagonist is administered at the same time as the additional therapeutic agent. In some instances, the PD-1 axis binding antagonist is in a separate composition as the additional therapeutic agent. In some instances, the PD-1 axis binding antagonist is in the same composition as the additional therapeutic agent. In some instances, the PD-1 axis binding antagonist is administered through a separate intravenous line from any other therapeutic agent administered to the individual on the same day.

The PD-1 axis binding antagonist and any additional therapeutic agent(s) may be administered by the same route of administration or by different routes of administration. In some instances, the PD-1 axis binding antagonist is administered intravenously, intramuscularly, subcutaneously, topically, orally, transdermally, intraperitoneally, intraorbitally, by implantation, by inhalation, intrathecally, intraventricularly, or intranasally. In some instances, the additional therapeutic agent is administered intravenously, intramuscularly, subcutaneously, topically, orally, transdermally, intraperitoneally, intraorbitally, by implantation, by inhalation, intrathecally, intraventricularly, or intranasally.

In a preferred embodiment, the PD-1 axis binding antagonist is administered intravenously. In one example, atezolizumab may be administered intravenously over 60 minutes; if the first infusion is tolerated, all subsequent infusions may be delivered over 30 minutes. In some instances, the PD-1 axis binding antagonist is not administered as an intravenous push or bolus. In another preferred embodiment, the PD-1 axis binding antagonist is administered subcutaneously.

Also provided herein are methods for treating NSCLC in an individual comprising administering to the individual a treatment regimen comprising an effective amount of a PD-1 axis binding antagonist (e.g., atezolizumab) in combination with another anti-cancer agent or cancer therapy. For example, a PD-1 axis binding antagonist may be administered in combination with an additional chemotherapy or chemotherapeutic agent (see definition above); a targeted therapy or targeted therapeutic agent; an immunotherapy or immunotherapeutic agent, for example, a monoclonal antibody; one or more cytotoxic agents (see definition above); or combinations thereof. For example, the PD-1 axis binding antagonist may be administered in combination with bevacizumab, paclitaxel, paclitaxel protein-bound (e.g., nab-paclitaxel), carboplatin, cisplatin, pemetrexed, gemcitabine, etoposide, cobimetinib, vemurafenib, or a combination thereof. The PD-1 axis binding antagonist may be an anti-PD-L1 antibody (e.g., atezolizumab) or an anti-PD-1 antibody. For example, in some instances, the PD-1 axis binding antagonist may be administered in combination with bevacizumab, paclitaxel, and carboplatin. In other instances, the PD-1 axis binding antagonist may be administered in combination with nab-paclitaxel and carboplatin.

In some instances, the treatment may further comprise an additional therapy. Any suitable additional therapy known in the art or described herein may be used. The additional therapy may be radiation therapy, surgery, gene therapy, DNA therapy, viral therapy, RNA therapy, immunotherapy, bone marrow transplantation, nanotherapy, monoclonal antibody therapy, gamma irradiation, or a combination of the foregoing.

In some instances, the additional therapy is the administration of side-effect limiting agents (e.g., agents intended to lessen the occurrence and/or severity of side effects of treatment, such as anti-nausea agents, a corticosteroid (e.g., prednisone or an equivalent, e.g., at a dose of 1-2 mg/kg/day), hormone replacement medicine(s), and the like).

### III. Assessment of PD-L1 Expression

The expression of PD-L1 may be assessed in a patient treated according to any of the methods and compositions for use described herein. The methods and compositions for use may include determining the expression level of PD-L1 in a biological sample (e.g., a tumor sample) obtained from the patient. In other examples, the expression level of PD-L1 in a biological sample (e.g., a tumor sample) obtained from the patient has been determined prior to initiation of treatment or after initiation of treatment. PD-L1 expression may be determined using any suitable approach. For example, PD-L1 expression may be determined as described in U.S. patent application Ser. Nos. 15/787,988 and 15/790,680. Any suitable tumor sample may be used, e.g., a formalin-fixed and paraffin-embedded (FFPE) tumor sample, an archival tumor sample, a fresh tumor sample, or a frozen tumor sample.

For example, PD-L1 expression may be determined in terms of the percentage of a tumor sample comprised by tumor-infiltrating immune cells expressing a detectable expression level of PD-L1, as the percentage of tumor-infiltrating immune cells in a tumor sample expressing a detectable expression level of PD-L1, and/or as the percentage of tumor cells in a tumor sample expressing a detectable expression level of PD-L1. It is to be understood that in any of the preceding examples, the percentage of the tumor sample comprised by tumor-infiltrating immune cells may be in terms of the percentage of tumor area covered by tumor-infiltrating immune cells in a section of the tumor sample obtained from the patient, for example, as assessed by IHC using an anti-PD-L1 antibody (e.g., the SP263 antibody). Any suitable anti-PD-L1 antibody may be used, including, e.g., SP142 (Ventana), SP263 (Ventana), 22C3 (Dako), 28-8 (Dako), E1L3N (Cell Signaling Technology), 4059 (ProSci, Inc.), h5H1 (Advanced Cell Diagnostics), and 9A11. In some examples, the anti-PD-L1 antibody is SP263. In other examples, the anti-PD-L1 antibody is SP142. In yet other examples, the anti-PD-L1 antibody is 22C3.

In some examples, a tumor sample obtained from the patient has a detectable expression level of PD-L1 in less than 1% of the tumor cells in the tumor sample, in 1% or more of the tumor cells in the tumor sample, in from 1% to less than 5% of the tumor cells in the tumor sample, in 5% or more of the tumor cells in the tumor sample, in from 5% to less than 50% of the tumor cells in the tumor sample, or in 50% or more of the tumor cells in the tumor sample.

In some examples, a tumor sample obtained from the patient has a detectable expression level of PD-L1 in tumor-infiltrating immune cells that comprise less than 1% of the tumor sample, more than 1% of the tumor sample, from 1% to less than 5% of the tumor sample, more than 5% of the tumor sample, from 5% to less than 10% of the tumor sample, or more than 10% of the tumor sample.

In some examples, tumor samples may be scored for PD-L1 positivity in tumor-infiltrating immune cells and/or in tumor cells according to the criteria for diagnostic assessment shown in Table 1 and/or Table 2, respectively. For example, the criteria for diagnostic assessment shown in Table 1 and/or Table 2 may be used for the SP142 antibody, e.g., in a Ventana SP142 IHC assay.

TABLE 1
Tumor-infiltrating immune cell (IC) IHC diagnostic criteria

| PD-L1 Diagnostic Assessment | IC Score |
|---|---|
| Absence of any discernible PD-L1 staining | IC0 |
| OR | |
| Presence of discernible PD-L1 staining of any | |
| intensity in tumor-infiltrating immune cells | |
| covering <1% of tumor area occupied by tumor cells, | |
| associated intratumoral stroma, and contiguous | |
| peri-tumoral desmoplastic stroma | |
| Presence of discernible PD-L1 staining of any | IC1 |
| intensity in tumor-infiltrating immune cells | |
| covering ≥1% to <5% of tumor area occupied by tumor | |
| cells, associated intratumoral stroma, and contiguous | |
| peri-tumoral desmoplastic stroma | |
| Presence of discernible PD-L1 staining of any | IC2 |
| intensity in tumor-infiltrating immune cells | |
| covering ≥5% to <10% of tumor area occupied by | |
| tumor cells, associated intratumoral stroma, and | |
| contiguous peri-tumoral desmoplastic stroma | |
| Presence of discernible PD-L1 staining of any | IC3 |
| intensity in tumor-infiltrating immune cells | |
| covering ≥10% of tumor area occupied by tumor | |
| cells, associated intratumoral stroma, and | |
| contiguous peri-tumoral desmoplastic stroma | |

TABLE 2
Tumor cell (TC) IHC diagnostic criteria

| PD-L1 Diagnostic Assessment | TC Score |
|---|---|
| Absence of any discernible PD-L1 staining | TC0 |
| OR | |
| Presence of discernible PD-L1 staining of any | |
| intensity in <1% of tumor cells | |
| Presence of discernible PD-L1 staining of any | TC1 |
| intensity in ≥1% to <5% of tumor cells | |
| Presence of discernible PD-L1 staining of any | TC2 |
| intensity in ≥5% to <50% of tumor cells | |
| Presence of discernible PD-L1 staining of any | TC3 |
| intensity in ≥50% of tumor cells | |

In some instances, a tumor sample obtained from the patient has a detectable protein expression level of PD-L1. In some instances, the detectable protein expression level of PD-L1 has been determined by an IHC assay. In some instances, the IHC assay uses anti-PD-L1 antibody SP263 (e.g., the Ventana SP263 IHC assay). In some instances, the tumor sample has been determined to have a PD-L1-positive tumor cell fraction greater than, or equal to, 50% of the tumor sample. In some instances, the tumor sample has been determined to have a PD-L1-positive tumor cell fraction less than 50% of the tumor sample. In some instances, the tumor sample has been determined to have a PD-L1-positive tumor cell fraction greater than, or equal to, 1% of the tumor sample. In some instances, the tumor sample has been determined to have a PD-L1-positive tumor cell fraction greater than, or equal to, 1% and less than 50% of the tumor sample.

For example, in some examples, a tumor sample obtained from the patient has a detectable expression level of PD-L1 in about 1% or more of the tumor cells in the tumor sample, e.g., as assessed using the SP263 antibody, e.g., in a Ventana SP263 IHC assay. In some examples, a tumor sample obtained from the patient has been determined to have a detectable expression level of PD-L1 in tumor-infiltrating immune cells that comprise about 50% or more of the tumor sample, e.g., as assessed using the SP263 antibody, e.g., in a Ventana SP263 IHC assay.

In some instances, the IHC assay uses the anti-PD-L1 antibody 22C3. In some instances, the IHC assay is the pharmDx 22C3 IHC assay. In some instances, the PD-L1-positive tumor cell fraction is greater than, or equal to, 50% as determined by positive staining with the anti-PD-L1 antibody 22C3. In some embodiments, the tumor sample has been determined to have a combined positive score (CPS) of greater than, or equal to, 10 or a tumor proportion score (TPS) of greater than or equal to 1% in the tumor sample, e.g., as determined using the anti-PD-L1 antibody 22C3 as part of the pharmDx 22C3 IHC assay. In some embodiments, the tumor sample has been determined to have a CPS of greater than, or equal to, 10 or a TPS of greater than or equal to 1% and less than 50% in the tumor sample, e.g., as determined using the anti-PD-L1 antibody 22C3 as part of the pharmDx 22C3 IHC assay. In some embodiments, the tumor sample has been determined to have a CPS of greater than, or equal to, 20 or a TPS of greater than or equal to 50% in the tumor sample, e.g., as determined using the anti-PD-L1 antibody 22C3 as part of the pharmDx 22C3 IHC assay.

In some instances, the IHC assay uses the anti-PD-L1 antibody 28-8. In some instances, the IHC assay is the pharmDx 28-8 IHC assay. In some instances, the PD-L1-positive tumor cell fraction is greater than, or equal to, 50% as determined by positive staining with the anti-PD-L1 antibody 28-8.

### IV. PD-1 Axis Binding Antagonists

PD-1 axis binding antagonists may include PD-L1 binding antagonists, PD-1 binding antagonists, and PD-L2 binding antagonists. Any suitable PD-1 axis binding antagonist may be used.

### A. PD-L1 Binding Antagonists

In some instances, the PD-L1 binding antagonist inhibits the binding of PD-L1 to one or more of its ligand binding partners. In other instances, the PD-L1 binding antagonist inhibits the binding of PD-L1 to PD-1. In yet other instances, the PD-L1 binding antagonist inhibits the binding of PD-L1 to B7-1. In some instances, the PD-L1 binding antagonist inhibits the binding of PD-L1 to both PD-1 and B7-1. The PD-L1 binding antagonist may be, without limitation, an antibody, an antigen-binding fragment thereof, an immunoadhesin, a fusion protein, an oligopeptide, or a small molecule. In some instances, the PD-L1 binding antagonist is a small molecule that inhibits PD-L1 (e.g., GS-4224, INCB086550, MAX-10181, INCB090244, CA-170, or ABSK041). In some instances, the PD-L1 binding antagonist is a small molecule that inhibits PD-L1 and VISTA. In some instances, the PD-L1 binding antagonist is CA-170 (also known as AUPM-170). In some instances, the PD-L1 binding antagonist is a small molecule that inhibits PD-L1 and TIM3. In some instances, the small molecule is a compound described in WO 2015/033301 and/or WO 2015/033299.

In some instances, the PD-L1 binding antagonist is an anti-PD-L1 antibody. A variety of anti-PD-L1 antibodies are contemplated and described herein. In any of the instances herein, the isolated anti-PD-L1 antibody can bind to a human PD-L1, for example a human PD-L1 as shown in UniProtKB/Swiss-Prot Accession No. Q9NZQ7-1, or a variant thereof. In some instances, the anti-PD-L1 antibody is capable of inhibiting binding between PD-L1 and PD-1 and/or between PD-L1 and B7-1. In some instances, the anti-PD-L1 antibody is a monoclonal antibody. In some instances, the anti-PD-L1 antibody is an antibody fragment selected from the group consisting of Fab, Fab′-SH, Fv, scFv, and (Fab′) 2 fragments. In some instances, the anti-PD-L1 antibody is a humanized antibody. In some instances, the anti-PD-L1 antibody is a human antibody. Exemplary anti-PD-L1 antibodies include atezolizumab, MDX-1105, MEDI4736 (durvalumab), MSB0010718C (avelumab), SHR-1316, CS1001, envafolimab, TQB2450, ZKAB001, LP-002, CX-072, IMC-001, KL-A167, APL-502, cosibelimab, lodapolimab, FAZ053, TG-1501, BGB-A333, BCD-135, AK-106, LDP, GR1405, HLX20, MSB2311, RC98, PDL-GEX, KD036, KY1003, YBL-007, and HS-636. Instances of anti-PD-L1 antibodies useful in the methods of this invention and methods of making them are described in International Patent Application Publication No. WO 2010/077634 and U.S. Pat. No. 8,217,149, each of which is incorporated herein by reference in its entirety.

In some instances, the anti-PD-L1 antibody comprises:
(a) an HVR-H1, HVR-H2, and HVR-H3 sequence of GFTFSDSWIH (SEQ ID NO: 3), AWISPYGGSTYYADSVKG (SEQ ID NO: 4) and RHWPGGFDY (SEQ ID NO: 5), respectively, and
(b) an HVR-L1, HVR-L2, and HVR-L3 sequence of RASQDVSTAVA (SEQ ID NO: 6), SASFLYS (SEQ ID NO: 7) and QQYLYHPAT (SEQ ID NO: 8), respectively.

In one embodiment, the anti-PD-L1 antibody comprises:
(a) a heavy chain variable region (VH) comprising the amino acid sequence: EVQLVESGGGLVQPGGSLRLSCAASGFTFSDSWIHWVRQAPGKGLEWVAWISPYGGSTYYADSVKGRF TISADTSKNTAYLQMNSLRAEDTAVYYCARRHWPGGFDYWGQGTLVTVSS (SEQ ID NO: 9), and
(b) the light chain variable region (VL) comprising the amino acid sequence:

(SEQ ID NO: 10)
DIQMTQSPSSLSASVGDRVTITCRASQDVSTAVAWYQQKPGKAPKLLIYSA
SFLYSGVPSRFSGSGSGTDFTLTISSLQPEDFATYYCQQYLYHPATFGQGT
KVEIKR.

In some instances, the anti-PD-L1 antibody comprises (a) a VH comprising an amino acid sequence comprising having at least 95% sequence identity (e.g., at least 95%, 96%, 97%, 98%, or 99% sequence identity) to, or the sequence of SEQ ID NO: 9; (b) a VL comprising an amino acid sequence comprising having at least 95% sequence identity (e.g., at least 95%, 96%, 97%, 98%, or 99% sequence identity) to, or the sequence of SEQ ID NO: 10; or (c) a VH as in (a) and a VL as in (b).

In one embodiment, the anti-PD-L1 antibody comprises atezolizumab, which comprises:
(a) the heavy chain amino acid sequence: EVOLVESGGGLVQPGGSLRLSCAASGFTFSDSWIHWVRQAPGKGLEWVAWISPYGGSTYYADSVKGRF TISADTSKNTAYLQMNSLRAEDTAVYYCARRHWPGGFDYWGQGTLVTVSSASTKGPSVFPLAPSSKSTS GGTAALGCLVKDYFPEPVTVSWNSGALTSGVHTFPAVLQSSGLYSLSSVVTVPSSSLGTQTYICNVNHKP SNTKVDKKVEPKSCDKTHTCPPCPAPELLGGPSVFLFPPKPKDTLMISRTPEVTCVVVDVSHEDPEVKFN WYVDGVEVHNAKTKPREEQYASTYRVVSVLTVLHQDWLNGKEYKCKVSNKALPAPIEKTISKAKGQPRE PQVYTLPPSREEMTKNQVSLTCLVKGFYPSDIAVEWESNGQPENNYKTTPPVLDSDGSFFLYSKLTVDKS RWQQGNVFSCSVMHEALHNHYTQKSLSLSPG (SEQ ID NO: 1), and
(b) the light chain amino acid sequence:

(SEQ ID NO: 2)
DIQMTQSPSSLSASVGDRVTITCRASQDVSTAVAWYQQKPGKAPKLLIYSA
SFLYSGVPSRFSGSGSGTDFTLTISSLOPEDFATYYCQQYLYHPATFGQGT
KVEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSP
VTKSFNRGEC.

In some instances, the anti-PD-L1 antibody is avelumab (CAS Registry Number: 1537032-82-8). Avelumab, also known as MSB0010718C, is a human monoclonal IgG1 anti-PD-L1 antibody (Merck KGaA, Pfizer).

In some instances, the anti-PD-L1 antibody is durvalumab (CAS Registry Number: 1428935-60-7). Durvalumab, also known as MEDI4736, is an Fc-optimized human monoclonal IgG1 kappa anti-PD-L1 antibody (MedImmune, AstraZeneca) described in WO 2011/066389 and US 2013/034559.

In some instances, the anti-PD-L1 antibody is MDX-1105 (Bristol Myers Squibb). MDX-1105, also known as BMS-936559, is an anti-PD-L1 antibody described in WO 2007/005874.

In some instances, the anti-PD-L1 antibody is LY3300054 (Eli Lilly).

In some instances, the anti-PD-L1 antibody is STI-A1014 (Sorrento). STI-A1014 is a human anti-PD-L1 antibody.

In some instances, the anti-PD-L1 antibody is KN035 (Suzhou Alphamab). KN035 is single-domain antibody (dAB) generated from a camel phage display library.

In some instances, the anti-PD-L1 antibody comprises a cleavable moiety or linker that, when cleaved (e.g., by a protease in the tumor microenvironment), activates an antibody antigen binding domain to allow it to bind its antigen, e.g., by removing a non-binding steric moiety. In some instances, the anti-PD-L1 antibody is CX-072 (CytomX Therapeutics).

In some instances, the anti-PD-L1 antibody comprises the six HVR sequences (e.g., the three heavy chain HVRs and the three light chain HVRs) and/or the heavy chain variable domain and light chain variable domain from an anti-PD-L1 antibody described in US20160108123, WO 2016/000619, WO 2012/145493, U.S. Pat. No. 9,205,148, WO 2013/181634, or WO 2016/061142.

In a still further specific aspect, the anti-PD-L1 antibody has reduced or minimal effector function.

In a still further specific aspect, the minimal effector function results from an “effector-less Fc mutation” or aglycosylation mutation. In still a further instance, the effector-less Fc mutation is an N297A or D265A/N297A substitution in the constant region. In still a further instance, the effector-less Fc mutation is an N297A substitution in the constant region. In some instances, the isolated anti-PD-L1 antibody is aglycosylated. Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used. Removal of glycosylation sites from an antibody is conveniently accomplished by altering the amino acid sequence such that one of the above-described tripeptide sequences (for N-linked glycosylation sites) is removed. The alteration may be made by substitution of an asparagine, serine or threonine residue within the glycosylation site with another amino acid residue (e.g., glycine, alanine, or a conservative substitution).

### B. PD-1 Binding Antagonists

In some instances, the PD-1 axis binding antagonist is a PD-1 binding antagonist. For example, in some instances, the PD-1 binding antagonist inhibits the binding of PD-1 to one or more of its ligand binding partners. In some instances, the PD-1 binding antagonist inhibits the binding of PD-1 to PD-L1. In other instances, the PD-1 binding antagonist inhibits the binding of PD-1 to PD-L2. In yet other instances, the PD-1 binding antagonist inhibits the binding of PD-1 to both PD-L1 and PD-L2. The PD-1 binding antagonist may be, without limitation, an antibody, an antigen-binding fragment thereof, an immunoadhesin, a fusion protein, an oligopeptide, or a small molecule. In some instances, the PD-1 binding antagonist is an immunoadhesin (e.g., an immunoadhesin comprising an extracellular or PD-1 binding portion of PD-L1 or PD-L2 fused to a constant region (e.g., an Fc region of an immunoglobulin sequence). For example, in some instances, the PD-1 binding antagonist is an Fc-fusion protein. In some instances, the PD-1 binding antagonist is AMP-224. AMP-224, also known as B7-DClg, is a PD-L2-Fc fusion soluble receptor described in WO 2010/027827 and WO 2011/066342. In some instances, the PD-1 binding antagonist is a peptide or small molecule compound. In some instances, the PD-1 binding antagonist is AUNP-12 (PierreFabre/Aurigene). See, e.g., WO 2012/168944, WO 2015/036927, WO 2015/044900, WO 2015/033303, WO 2013/144704, WO 2013/132317, and WO 2011/161699. In some instances, the PD-1 binding antagonist is a small molecule that inhibits PD-1.

In some instances, the PD-1 binding antagonist is an anti-PD-1 antibody. A variety of anti-PD-1 antibodies can be utilized in the methods and uses disclosed herein. In any of the instances herein, the PD-1 antibody can bind to a human PD-1 or a variant thereof. In some instances the anti-PD-1 antibody is a monoclonal antibody. In some instances, the anti-PD-1 antibody is an antibody fragment selected from the group consisting of Fab, Fab′, Fab′-SH, Fv, scFv, and (Fab′) 2 fragments. In some instances, the anti-PD-1 antibody is a humanized antibody. In other instances, the anti-PD-1 antibody is a human antibody. Exemplary anti-PD-1 antagonist antibodies include nivolumab, pembrolizumab, MEDI-0680, PDR001 (spartalizumab), REGN2810 (cemiplimab), BGB-108, prolgolimab, camrelizumab, sintilimab, tislelizumab, toripalimab, dostarlimab, retifanlimab, sasanlimab, penpulimab, CS1003, HLX10, SCT-110A, zimberelimab, balstilimab, genolimzumab, BI 754091, cetrelimab, YBL-006, BAT1306, HX008, budigalimab, AMG 404, CX-188, JTX-4014, 609A, Sym021, LZM009, F520, SG001, AM0001, ENUM 244C8, ENUM 388D4, STI-1110, AK-103, and hAb21.

In some instances, the anti-PD-1 antibody is nivolumab (CAS Registry Number: 946414-94-4). Nivolumab (Bristol-Myers Squibb/Ono), also known as MDX-1106-04, MDX-1106, ONO-4538, BMS-936558, and OPDIVO®, is an anti-PD-1 antibody described in WO 2006/121168.

In some instances, the anti-PD-1 antibody is pembrolizumab (CAS Registry Number: 1374853-91-4). Pembrolizumab (Merck), also known as MK-3475, Merck 3475, lambrolizumab, SCH-900475, and KEYTRUDA®, is an anti-PD-1 antibody described in WO 2009/114335.

In some instances, the anti-PD-1 antibody is MEDI-0680 (AMP-514; AstraZeneca). MEDI-0680 is a humanized IgG4 anti-PD-1 antibody.

In some instances, the anti-PD-1 antibody is PDR001 (CAS Registry No. 1859072-53-9; Novartis). PDR001 is a humanized IgG4 anti-PD-1 antibody that blocks the binding of PD-L1 and PD-L2 to PD-1.

In some instances, the anti-PD-1 antibody is REGN2810 (Regeneron). REGN2810 is a human anti-PD-1 antibody.

In some instances, the anti-PD-1 antibody is BGB-108 (BeiGene).

In some instances, the anti-PD-1 antibody is BGB-A317 (BeiGene).

In some instances, the anti-PD-1 antibody is JS-001 (Shanghai Junshi). JS-001 is a humanized anti-PD-1 antibody.

In some instances, the anti-PD-1 antibody is STI-A1110 (Sorrento). STI-A1110 is a human anti-PD-1 antibody.

In some instances, the anti-PD-1 antibody is INCSHR-1210 (Incyte). INCSHR-1210 is a human IgG4 anti-PD-1 antibody.

In some instances, the anti-PD-1 antibody is PF-06801591 (Pfizer).

In some instances, the anti-PD-1 antibody is TSR-042 (also known as ANB011; Tesaro/AnaptysBio).

In some instances, the anti-PD-1 antibody is AM0001 (ARMO Biosciences).

In some instances, the anti-PD-1 antibody is ENUM 244C8 (Enumeral Biomedical Holdings). ENUM 244C8 is an anti-PD-1 antibody that inhibits PD-1 function without blocking binding of PD-L1 to PD-1.

In some instances, the anti-PD-1 antibody is ENUM 388D4 (Enumeral Biomedical Holdings). ENUM 388D4 is an anti-PD-1 antibody that competitively inhibits binding of PD-L1 to PD-1.

In some instances, the anti-PD-1 antibody comprises the six HVR sequences (e.g., the three heavy chain HVRs and the three light chain HVRs) and/or the heavy chain variable domain and light chain variable domain from an anti-PD-1 antibody described in WO 2015/112800, WO 2015/112805, WO 2015/112900, US20150210769, WO2016/089873, WO 2015/035606, WO 2015/085847, WO 2014/206107, WO 2012/145493, U.S. Pat. No. 9,205,148, WO 2015/119930, WO 2015/119923, WO 2016/032927, WO 2014/179664, WO 2016/106160, and WO 2014/194302.

In a still further specific aspect, the anti-PD-1 antibody has reduced or minimal effector function. In a still further specific aspect, the minimal effector function results from an “effector-less Fc mutation” or aglycosylation mutation. In still a further instance, the effector-less Fc mutation is an N297A or D265A/N297A substitution in the constant region. In some instances, the isolated anti-PD-1 antibody is aglycosylated.

### C. PD-L2 Binding Antagonists

In some instances, the PD-1 axis binding antagonist is a PD-L2 binding antagonist. In some instances, the PD-L2 binding antagonist is a molecule that inhibits the binding of PD-L2 to its ligand binding partners. In a specific aspect, the PD-L2 binding ligand partner is PD-1. The PD-L2 binding antagonist may be, without limitation, an antibody, an antigen-binding fragment thereof, an immunoadhesin, a fusion protein, an oligopeptide, or a small molecule.

In some instances, the PD-L2 binding antagonist is an anti-PD-L2 antibody. In any of the instances herein, the anti-PD-L2 antibody can bind to a human PD-L2 or a variant thereof. In some instances, the anti-PD-L2 antibody is a monoclonal antibody. In some instances, the anti-PD-L2 antibody is an antibody fragment selected from the group consisting of Fab, Fab′, Fab′-SH, Fv, scFv, and (Fab′)₂ fragments. In some instances, the anti-PD-L2 antibody is a humanized antibody. In other instances, the anti-PD-L2 antibody is a human antibody. In a still further specific aspect, the anti-PD-L2 antibody has reduced or minimal effector function. In a still further specific aspect, the minimal effector function results from an “effector-less Fc mutation” or aglycosylation mutation. In still a further instance, the effector-less Fc mutation is an N297A or D265A/N297A substitution in the constant region. In some instances, the isolated anti-PD-L2 antibody is aglycosylated.

### V. Adjuvant Therapies

Provided herein are methods for treating a lung cancer (e.g., NSCLC) in an individual comprising administering to the individual a treatment regimen comprising a PD-1 axis binding antagonist (e.g., atezolizumab) following an adjuvant therapy. Further provided are methods for treating a lung cancer (e.g., NSCLC) in an individual comprising administering to the individual an adjuvant therapy followed by a treatment regimen comprising a PD-1 axis binding antagonist (e.g., atezolizumab). Also provided are related compositions (e.g., pharmaceutical compositions) for use, kits, and articles of manufacture. Any of the methods, compositions for use, kits, or articles of manufacture described herein may be used following any suitable adjuvant therapy.

It is to be understood that each of the methods provided herein (e.g., in Section II above) may be an adjuvant therapy that follows an earlier or initial adjuvant therapy, such as a platinum-based adjuvant therapy. In certain instances, provided herein is a method of adjuvant treatment following resection (e.g., complete resection) and platinum-based chemotherapy (e.g., up to 4 cycles of platinum-based chemotherapy) for an individual having a lung cancer (e.g., NSCLC). In certain instances, the individual has not had progression after platinum-based adjuvant chemotherapy.

For example, provided herein is a method of treating NSCLC in an individual that includes administering an effective amount of an adjuvant therapy to the individual, followed by administering an effective amount of a PD-1 axis binding antagonist (e.g., atezolizumab) to the individual. For example, any of the methods described in Section II above may be performed following the adjuvant therapy.

In some instances, any of the methods described in Section II above may be an adjuvant therapy that may be performed following an initial adjuvant therapy. In certain instances, any of the adjuvant therapies described below may be the initial adjuvant therapy. In some instances, an initial adjuvant therapy does not include a PD-1 axis binding antagonist (e.g., atezolizumab), and a subsequent adjuvant therapy includes a PD-1 axis binding antagonist (e.g., atezolizumab). In some instances, the initial adjuvant therapy is a platinum-based adjuvant therapy.

In one example, provided herein is a method of treating NSCLC in an individual, the method comprising administering to the individual a platinum-based adjuvant therapy followed by a treatment regimen comprising an effective amount of a PD-1 axis binding antagonist, wherein the treatment regimen extends the individual's DFS and/or OS as compared to BSC.

In another example, provided herein is a platinum-based adjuvant therapy for use in treatment of NSCLC in an individual, the treatment comprising administration of a platinum-based adjuvant therapy followed by a treatment regimen comprising an effective amount of a PD-1 axis binding antagonist, wherein the treatment regimen extends the individual's DFS and/or OS as compared to BSC.

In yet another example, provided herein is the use of a platinum-based adjuvant therapy in the manufacture of a medicament for use in treatment of NSCLC in an individual, the treatment comprising administration of a platinum-based adjuvant therapy followed by a treatment regimen comprising an effective amount of a PD-1 axis binding antagonist, wherein the treatment regimen extends the individual's DFS and/or OS as compared to BSC.

Any method as described herein (e.g., an adjuvant therapy comprising atezolizumab) may follow any suitable adjuvant therapy, such as any initial adjuvant therapy.

In some instances, the adjuvant therapy includes one or more chemotherapeutic agent, including a platinum-based chemotherapeutic agent (e.g., cisplatin or carboplatin), a vinca alkaloid (e.g., vinorelbine, vindesine, or vinblastine), a taxane (e.g., paclitaxel), an anti-metabolite (e.g., gemcitabine or pemetrexed), UFT (an oral fluorouracil (FU) derivative composed of tegafur and uracil), mitomycin, an alkylating agent (e.g., ifosfamide), a topoisomerase inhibitor (e.g., etoposide), or a combination thereof. In some instances, the adjuvant therapy includes a platinum-based adjuvant therapy. For example, the platinum-based adjuvant therapy may be any platinum-based adjuvant therapy described in Arriagada et al. N. Engl. J. Med. 350:351-360, 2004; Winton et al. N. Engl. J. Med. 352:2589-2597, 2005; Rosell et al. Lancet Oncol. 7:719-727, 2006; Scagliotti et al. J. Natl. Cancer Inst. 95:1453-1461, 2003; Waller et al. Eur. J. Cardiothorac. Surg. 26 (1): 173-182, 2004; Strauss et al. J. Clin. Oncol. 26:5043-5051, 2008; or Heon et al. Thorac. Cardiovasc. Surg. 144 (3): S39-42, 2012.

In some instances, the platinum-based adjuvant therapy comprises cisplatin. Cisplatin may be administered at any suitable dose. In some instances, cisplatin may be administered at a dose of about 75 mg/m² intravenously on Day 1 of each 21-day cycle.

For example, the platinum-based chemotherapy may include a platinum-based chemotherapeutic agent (e.g., cisplatin or carboplatin) and one or more additional chemotherapeutic agents. In some instances, the one or more additional chemotherapeutic agents comprises a vinca alkaloid, a taxane, an anti-metabolite, or a combination thereof. In some instances, the vinca alkaloid is vinorelbine. In some instances, the taxane is docetaxel. In some instances, the anti-metabolite is gemcitabine or pemetrexed. In some instances, the anti-metabolite is gemcitabine. In some instances, the anti-metabolite is pemetrexed.

In some instances, the platinum-based chemotherapy is a doublet that includes cisplatin and another chemotherapeutic agent (e.g., cisplatin/vinorelbine, cisplatin/vindesine, cisplatin/docetaxel, cisplatin/gemcitabine, or cisplatin/pemetrexed), or vinorelbine/cisplatin. In other instances, the platinum-based chemotherapy is a triplet (e.g., mitomycin/vindesine/cisplatin (MVP), mitomycin/ifosfamide/cisplatin (MIP), or mitomycin/vinblastine/cisplatin).

In some instances, the platinum-based adjuvant therapy includes up to eight 21-day cycles. For example, the platinum-based adjuvant therapy may comprise one, two, three, four, five, six, seven, or eight 21-day cycles. For example, the platinum-based adjuvant therapy may comprise 1 to 8, 2 to 8, 3 to 8, 4 to 8, 5 to 8, 6 to 8, 7 to 8, 1 to 7, 2 to 7, 3 to 7, 4 to 7, 5 to 7, 6 to 7, 1 to 6, 2 to 6, 3 to 6, 4 to 6, 5 to 6, 1 to 5, 2 to 5, 3 to 5, 4 to 5, 1 to 4, 2 to 4, 3 to 4, 1 to 3, 2 to 3, or 1 to 50 21-day cycles. In some instances, the platinum-based adjuvant therapy comprises up to 4 21-day cycles.

In some instances, the individual has had up to 4 cycles (e.g., 1 cycle, 2 cycle, 3 cycles, or 4 cycles) of a platinum-based adjuvant therapy, e.g., prior to administration of atezolizumab.

In some instances, the platinum-based adjuvant therapy comprises cisplatin and a vinca alkaloid, e.g., vinorelbine. In some instances, cisplatin is administered at a dose of about 75 mg/m² intravenously on Day 1 of each 21-day cycle, and vinorelbine is administered at a dose of about 30 mg/m² by intravenous push on Days 1 and 8 of each 21-day cycle.

In some instances, the platinum-based adjuvant therapy comprises cisplatin and a taxane, e.g., docetaxel. In some instances, cisplatin is administered at a dose of about 75 mg/m² intravenously on Day 1 of each 21-day cycle, and docetaxel is administered at a dose of about 75 mg/m² intravenously on Day 1 of each 21-day cycle.

In some instances, the platinum-based adjuvant therapy comprises cisplatin and an anti-metabolite, e.g., a nucleoside analogue such as gemcitabine. In some instances, cisplatin is administered at a dose of about 75 mg/m² intravenously on Day 1 of each 21-day cycle, and gemcitabine is administered at a dose of about 1250 mg/m² intravenously on Days 1 and 8 of each 21-day cycle.

In some instances, the platinum-based adjuvant therapy comprises cisplatin and an anti-metabolite, e.g., a folate anti-metabolite such as pemetrexed. In some instances, cisplatin is administered at a dose of about 75 mg/m² intravenously on Day 1 of each 21-day cycle, and pemetrexed is administered at a dose of about 500 mg/m² intravenously on Day 1 of each 21-day cycle. In some instances, the NSCLC is non-squamous NSCLC.

### VI. Pharmaceutical Compositions and Formulations

Also provided herein are pharmaceutical compositions and formulations comprising a PD-1 axis binding antagonist (e.g., atezolizumab) and, optionally, a pharmaceutically acceptable carrier. Also provided herein are pharmaceutical compositions and formulations comprising one or more additional therapeutic agents (e.g., one or more additional therapeutic agents used in an adjuvant therapy as described herein).

Pharmaceutical compositions and formulations as described herein can be prepared by mixing the active ingredients (e.g., a PD-1 axis binding antagonist) having the desired degree of purity with one or more optional pharmaceutically acceptable carriers (see, e.g., Remington's Pharmaceutical Sciences 16th edition, Osol, A. Ed. (1980)), e.g., in the form of lyophilized formulations or aqueous solutions.

An exemplary atezolizumab formulation comprises glacial acetic acid, L-histidine, polysorbate 20, and sucrose, with a pH of 5.8. For example, atezolizumab may be provided in a 20 mL vial containing 1200 mg of atezolizumab that is formulated in glacial acetic acid (16.5 mg), L-histidine (62 mg), polysorbate 20 (8 mg), and sucrose (821.6 mg), with a pH of 5.8. In another example, atezolizumab may be provided in a 14 mL vial containing 840 mg of atezolizumab that is formulated in glacial acetic acid (11.5 mg), L-histidine (43.4 mg), polysorbate 20 (5.6 mg), and sucrose (575.1 mg) with a pH of 5.8.

In some instances, atezolizumab is in the formulation at a concentration of about 125 g/L. In some embodiments, the L-histidine is at a concentration of about 20 mM. In some instances, the sucrose is at a concentration of about 240 mM. In some instances, the polysorbate is at a concentration of about 0.02% (w/v). In some instances, a formulation comprising atezolizumab is mixed with a hyaluronidase enzyme prior to being administered to a subject. In some instances, the hyaluronidase enzyme is recombinant human hyaluronidase (rHuPH20). In some instances, the hyaluronidase enzyme concentration in the mixture is about 2000 U/ml. In some instances, atezolizumab is not subject to prior lyophilization in a pharmaceutical composition described herein. Additional exemplary formulations for an anti-PD-L1 antibody (e.g., atezolizumab) are found in PCT Publication No. WO 2021/118930, which is incorporated herein by reference in its entirety.

### VII. Articles of Manufacture or Kits

In one aspect, provided herein is an article of manufacture or a kit for identifying an individual having a lung cancer (e.g., an NSCLC) who is likely to have an increased clinical benefit from an adjuvant treatment comprising a PD-1 axis binding antagonist (e.g., an anti-PD-L1 antibody, e.g., atezolizumab) and/or one or more additional therapeutic agents (e.g., one or more additional therapeutic agents used in an adjuvant therapy as described herein). In some instances, the article of manufacture or kit comprises reagents for determining the expression level of a TGFβ CAF gene signature in a tumor tissue sample obtained from the individual. In some instances, an expression level of LRRC15 that is at or above a reference LRRC15 expression level identifies that the individual as one who is likely to have an increased clinical benefit from the adjuvant treatment comprising the PD-1 axis binding antagonist (e.g., the anti-PD-L1 antibody, e.g., atezolizumab) compared to treatment with BSC. In some instances, the article of manufacture or kit comprises reagents for determining the expression level of LRRC15 in a tumor tissue sample obtained from the individual. In some instances, an expression level of LRRC15 that is at or above a reference LRRC15 expression level identifies that the individual as one who is likely to have an increased clinical benefit from the adjuvant treatment comprising the PD-1 axis binding antagonist (e.g., the anti-PD-L1 antibody, e.g., atezolizumab) compared to treatment with BSC.

In some aspects, the reagents are for determining the RNA expression levels of a set of genes comprising the TGFβ CAF gene signature. In some aspects, the reagents are for determining the RNA expression levels of LRRC15, FAP, COL11A1, COL10A1, FNDC1, GREM1, ITGA11, WISP1, PLPP4, PODNL1, TWIST1, COL8A2, SUGCT, NOX4, ZNF469, TENM3, P4HA3, ADAMTS12, and/or CPZ. In some aspects, the reagents are for determining the RNA expression levels of LRRC15, FAP, COL11A1, COL10A1, FNDC1, GREM1, ITGA11, WISP1, PLPP4, PODNL1, TWIST1, COL8A2, SUGCT, NOX4, ZNF469, TENM3, P4HA3, ADAMTS12, and CPZ. In some aspects, the reagents are for determining the RNA expression level of LRRC15. In some aspects, the article of manufacture or kit comprises reagents for performing RNA sequencing. In some aspects, the reagents are for performing RNA sequencing on an Illumina sequencing-by-synthesis platform (e.g., a NOVASEQ® 6000 platform). In some aspects, the reagents are for determining the protein expression levels of a set of genes comprising the TGFβ CAF gene signature. In some aspects, the reagents are for determining the protein expression level of LRRC15. In some aspects, the reagents are for performing a method selected from the group consisting of FACS, Western blot, ELISA, immunoprecipitation, immunohistochemistry, immunofluorescence, radioimmunoassay, dot blotting, immunodetection methods, HPLC, surface plasmon resonance, optical spectroscopy, mass spectrometry, HPLC, qPCR, RT-qPCR, multiplex qPCR or RT-qPCR, RNA-seq, microarray analysis, SAGE, MassARRAY technique, and FISH, and combinations thereof. In some aspects, the reagents are for performing immunohistochemistry platform (e.g., a Ventana staining platform such as a Ventana Discovery Ultra staining platform).

In another aspect, provided herein is an article of manufacture or a kit comprising a PD-1 axis binding antagonist (e.g., an anti-PD-L1 antibody, e.g., atezolizumab) and/or one or more additional therapeutic agents (e.g., one or more additional therapeutic agents used in an adjuvant therapy as described herein). In some instances, the article of manufacture or kit further comprises package insert comprising instructions for using the PD-1 axis binding antagonist to treat or delay recurrence or progression of lung cancer (e.g., NSCLC) in an individual, wherein a tumor tissue sample obtained from the individual has been determined to have an expression level of an TGFβ CAF gene signature that is at or above a reference TGFβ CAF gene signature expression level or a tumor tissue sample obtained from the individual has been determined to have an expression level of LRRC15 has been determined to be at or above a reference LRRC15 expression level, thereby indicating that the individual is likely to have an increased clinical benefit from treatment with the PD-1 axis binding antagonist (e.g., the anti-PD-L1 antibody, e.g., atezolizumab) compared to treatment with best supportive care (BSC). In some instances, the article of manufacture or kit further comprises package insert comprising instructions for using the PD-1 axis binding antagonist in combination with following an adjuvant therapy (e.g., a platinum-based adjuvant therapy) to treat or delay recurrence or progression of lung cancer (e.g., NSCLC) in an individual. Any of the PD-1 axis binding antagonists and/or other therapeutic agents described herein may be included in the article of manufacture or kits.

In some instances, the PD-1 axis binding antagonist and the one or more optional additional therapeutic agents are in the same container or separate containers. Suitable containers include, for example, bottles, vials, bags and syringes. The container may be formed from a variety of materials such as glass, plastic (such as polyvinyl chloride or polyolefin), or metal alloy (such as stainless steel or hastelloy). In some instances, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. In some instances, the article of manufacture further includes one or more of another agent (e.g., an additional chemotherapeutic agent or anti-neoplastic agent). Suitable containers for the one or more agent include, for example, bottles, vials, bags and syringes.

Any of the articles of manufacture or kits may include instructions to administer a PD-1 axis binding antagonist and/or one or more additional therapeutic agents (e.g., one or more additional therapeutic agents used in an adjuvant therapy as described herein) to an individual in accordance with any of the methods described herein, e.g., any of the methods set forth in Section II above.

In one example, provided herein is an article of manufacture comprising a PD-1 axis binding antagonist (e.g., an anti-PD-L1 antibody, e.g., atezolizumab) and instructions to administer the PD-1 axis binding antagonist (e.g., the anti-PD-L1 antibody, e.g., atezolizumab) as adjuvant treatment following resection and platinum-based chemotherapy for an individual with a lung cancer (e.g., NSCLC) whose tumor has been determined to have an expression level of an TGFβ CAF gene signature that is at or above a reference TGFβ CAF gene signature expression level. In one example, provided herein is an article of manufacture comprising a PD-1 axis binding antagonist (e.g., an anti-PD-L1 antibody, e.g., atezolizumab) and instructions to administer the PD-1 axis binding antagonist (e.g., the anti-PD-L1 antibody, e.g., atezolizumab) as adjuvant treatment following resection and platinum-based chemotherapy for an individual with NSCLC whose tumor has been determined to have an expression level of LRRC15 that is at or above a median expression level of LRRC15 in a reference population of individuals having stage IB to IIIA NSCLC. In some instances, the adjuvant treatment extends the individual's DFS as compared to BSC. In some instances, the adjuvant treatment extends the individual's OS as compared to BSC.

In one example, provided herein is an article of manufacture comprising atezolizumab and instructions to administer the atezolizumab in an adjuvant treatment following resection and platinum-based chemotherapy for an adult individual with stage IB-IIIA NSCLC (e.g., stage IB, stage IIA, stage IIB, or stage IIIA NSCLC), wherein the treatment comprises administration of an effective amount of a treatment regimen comprising atezolizumab, wherein the treatment regimen extends the individual's DFS and/or OS as compared to best supportive care, and wherein a tumor tissue sample obtained from the individual has been determined to have an expression level of an TGFβ CAF gene signature that is at or above a reference TGFβ CAF gene signature expression level. In one example, provided herein is an article of manufacture comprising atezolizumab and instructions to administer the atezolizumab in an adjuvant treatment following resection and platinum-based chemotherapy for an adult individual with stage IB-IIIA NSCLC (e.g., stage IB, stage IIA, stage IIB, or stage IIIA NSCLC), wherein the treatment comprises administration of an effective amount of a treatment regimen comprising atezolizumab, wherein the treatment regimen extends the individual's DFS and/or OS as compared to best supportive care, and wherein a tumor tissue sample obtained from the individual has been determined to have an expression level of LRRC15 that is at or above a median expression level of LRRC15 in a reference population of individuals having stage IB to IIIA NSCLC.

In one example, the PD-1 axis binding antagonist (e.g., the anti-PD-L1 antibody, e.g., atezolizumab) is administered to the individual as a monotherapy.

In another example, provided herein is an article of manufacture comprising atezolizumab for use in adjuvant treatment following complete resection and no progression after platinum-based chemotherapy for an adult individual with stage IB to IIIA NSCLC, wherein the atezolizumab is administered to the individual as a monotherapy, wherein the treatment regimen extends the individual's DFS and/or OS as compared to best supportive care, and wherein a tumor tissue sample obtained from the individual has been determined to have an expression level of an TGFβ CAF gene signature that is at or above a reference TGFβ CAF gene signature expression level. In another example, provided herein is an article of manufacture comprising atezolizumab for use in adjuvant treatment following complete resection and no progression after platinum-based chemotherapy for an adult individual with stage IB to IIIA NSCLC, wherein the atezolizumab is administered to the individual as a monotherapy, wherein the treatment regimen extends the individual's DFS and/or OS as compared to best supportive care, and wherein a tumor tissue sample obtained from the individual has been determined to have an expression level of LRRC15 that is at or above a median expression level of LRRC15 in a reference population of individuals having stage IB to IIIA NSCLC. In some embodiments, the NSCLC is stage II to IIIA.

In any of the preceding instances, the individual can have had up to 4 cycles of platinum-based chemotherapy.

In any of the preceding instances, atezolizumab may be administered to the individual intravenously at a dose of about 840 mg every 2 weeks, about 1200 mg every 3 weeks, or about 1680 mg every 4 weeks. In any of the preceding instances, atezolizumab may be administered to the individual subcutaneously at a dose of about 1875 mg every 3 weeks.

In any of the preceding instances, atezolizumab is administered to the individual for up to 1 year.

### EXAMPLES

### Example 1: IMpower010: Exploratory Analysis of Disease-Free Survival with Atezolizumab or Best Supportive Care by TGFβ Cancer-Associated Fibroblast Gene Signature Status in Individuals with Resected Non-Small Cell Lung Cancer

### Introduction

IMpower010 (NCT02486718) is a Phase III, multicenter, randomized study that showed significant and clinically meaningful disease-free survival (DFS) improvement with the PD-L1 inhibitor atezolizumab compared with best supportive care (BSC) in individuals with completely resected stage IB-IIIA NSCLC, including those whose tumors expressed PD-L1 on 1% or more of tumor cells (TC≥1% per SP263) (Felip E et al., Lancet, Vol 398, 1344-1357). Based on these data, atezolizumab was approved after adjuvant platinum-based chemotherapy for individuals with completely resected stage II-IIIA PD-L1 TC≥1% NSCLC in the US, China, Japan and other countries, and for individuals with completely resected stage II-IIIA PD-L1 TC≥50% NSCLC in the UK, Canada, Switzerland, Australia, Singapore, EU, and other countries (excluding EGFR mutations or ALK alterations). (European Medicines Agency. TECENTRIQ. Summary of Product Characteristics. 2022. 2022; Health Canada. TECENTRIQ Product Monograph. 2022. 2022; US Food and Drug Administration. TECENTRIQ. Highlights of Prescribing Information. 2022).

In this exploratory analysis, bulk RNA-seq based on a curated collection of gene signatures and other relevant biomarkers was used to examine what features may best predict atezolizumab DFS benefit compared with BSC in individuals with early-stage NSCLC.

### Results

Of 1005 individuals in the intention-to-treat (ITT) population, 500 had tissue available for RNA-seq and constituted the RNA-seq biomarker evaluable population (BEP). Reasons for exclusion from the RNA-seq BEP included no consent provided, insufficient tissue available, or sample failure. Baseline characteristics of the RNA-seq BEP were similar (<10% different) to those of the ITT population (Table 3).

TABLE 3
Baseline demographics and characteristics

| | ITT | RNA-seq | High TGFβ CAF | Low TGFβ CAF |
|---|---|---|---|---|
| | (n = 1005) | BEP (n = 500) | (n = 250) | (n = 250) |
| Median age (range), y | 62 (26-84) | 62 (26-84) | 62 (35-78) | 62 (26-84) |
| Male, n (%) | 672 (66.9) | 348 (69.6) | 179 (71.6) | 169 (67.6) |
| ECOG PS, n (%) | | | | |
| 0 | 556 (55.3) | 272 (54.4) | 136 (54.4) | 136 (54.4) |
| 1 | 446 (44.4) | 226 (45.2) | 113 (45.2) | 113 (45.2) |
| Tobacco history, n (%) | | | | |
| Current | 162 (16.1) | 100 (20.0) | 51 (20.4) | 49 (19.6) |
| Never | 222 (22.1) | 91(18.2) | 37 (14.8) | 54 (21.6) |
| Previous | 621 (61.8) | 309 (61.8) | 162 (64.8) | 147 (58.8) |
| Histology, n (%) | | | | |
| Non-squamous | 659 (65.6) | 298 (59.6) | 127 (50.8) | 171 (68.4) |
| Squamous | 346 (34.4) | 202 (40.4) | 123 (49.2) | 79 (31.6) |
| PD-L1 expression by | | | | |
| SP263, n (%) | | | | |
| TC <1% | 444 (44.2) | 237 (47.4) | 104 (41.6) | 133 (53.2) |
| TC 1%-49% | 277 (27.6) | 135 (27.0) | 76 (30.4) | 59 (23.6) |
| TC ≥50% | 258 (25.7) | 125 (25.0) | 67 (26.8) | 58 (23.2) |
| Stage, n (%) | | | | |
| IB | 123 (12.2) | 63 (12.6) | 24 (9.6) | 39 (15.6) |
| IIA | 295 (29.4) | 132 (26.4) | 77 (30.8) | 55 (22.0) |
| IIB | 174 (17.3) | 88 (17.6) | 43 (17.2) | 45 (18.0) |
| IIIA | 413 (41.1) | 217 (43.4) | 106 (42.4) | 111 (44.4) |

ECOG: Eastern Cooperative Oncology Group (Oken M M et al., Am J Clin Oncol. 1982 December; 5(6): 649-655); IC: tumor-infiltrating cells; IHC: immunohistochemistry; TC: tumor cells.

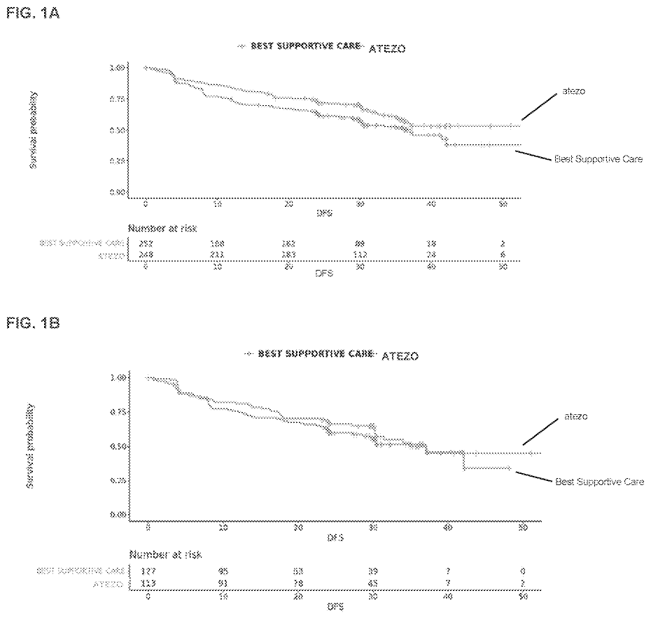

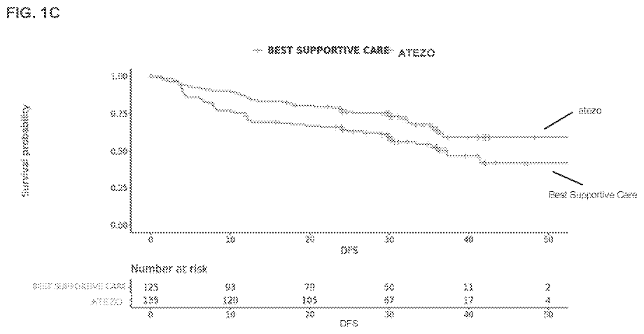

Based on data from the clinical cutoff date of Jan. 21, 2021, the DFS HR for atezolizumab vs BSC in RNA-seq BEP was 0.71 (95% CI: 0.54, 0.94). Median DFS was not reached (NR) in the atezolizumab arm and was 37.0 months in the BSC arm (FIG. 1A). For the PD-L1-positive (TC≥1% by SP263) and PD-L1-negative (TC<1% by SP263) subgroups of the RNA-seq BEP, the DFS HRs were 0.61 (95%: 0.41, 0.91) and 0.85 (95%: 0.58, 1.26), respectively (FIG. 1B and FIG. 1C).

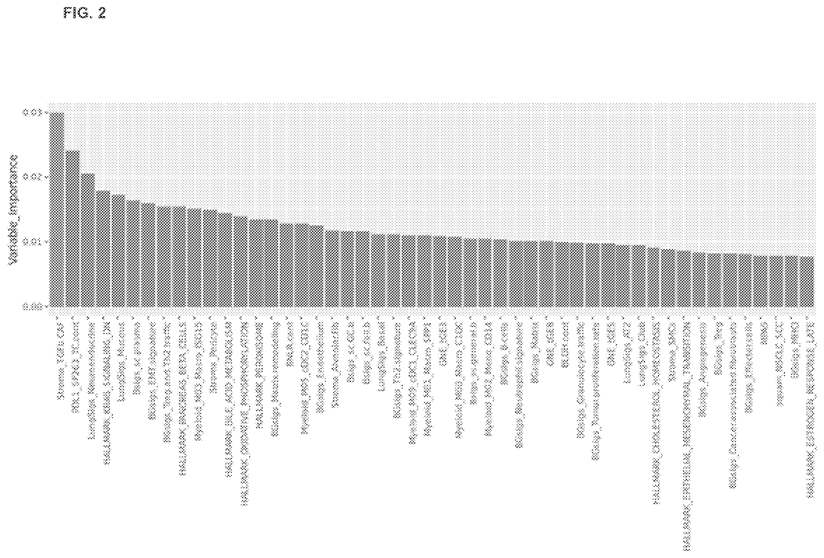

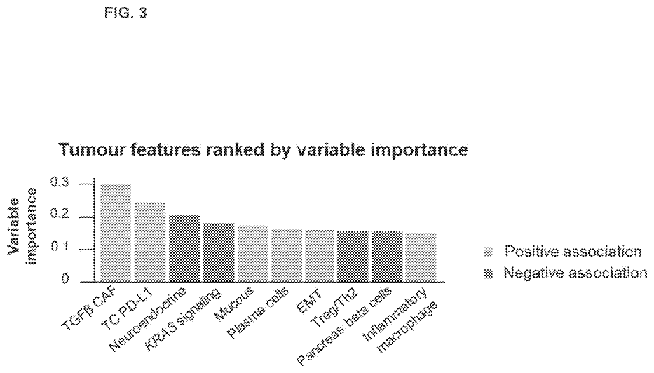

Using the generalized forests model, the TGFβ (transforming growth factor-beta) cancer-associated fibroblasts (CAF) gene signature was identified as having the greatest variable importance for a predictive association of DFS benefit with atezolizumab vs BSC, with SP263 TC (i.e., PD-L1 TC) having the second greatest variable importance (FIG. 2 and FIG. 3). The genes comprising the TGFβ CAF gene signature included: LRRC15, FAP, COL11A1, COL10A1, FNDC1, GREM1, ITGA11, WISP1, PLPP4, PODNL1, TWIST1, COL8A2, SUGCT, NOX4, ZNF469, TENM3, P4HA3, ADAMTS12 and CPZ.

Next, TGFβ CAF gene signature expression was assessed in all individuals in the RNA-seq BEP. Individuals were grouped into TGFβ CAF-low (<median; n=250) or TGFβ CAF-high (>median; n=250) subgroups. Baseline characteristics were generally well-balanced (<10% different) between the TGFβ CAF-low and -high subgroups. However, the prevalence of squamous histology was greater in the TGFβ CAF-high subgroup (n=123; 49%) compared with the TGFβ CAF-low subgroup (n=79; 32%), and the prevalence of individuals with PD-L1-negative tumors was greater in the TGFβ CAF-low subgroup (n=133; 53%) compared with the TGFβ CAF-high subgroup (n=104; 42%; Table 3).

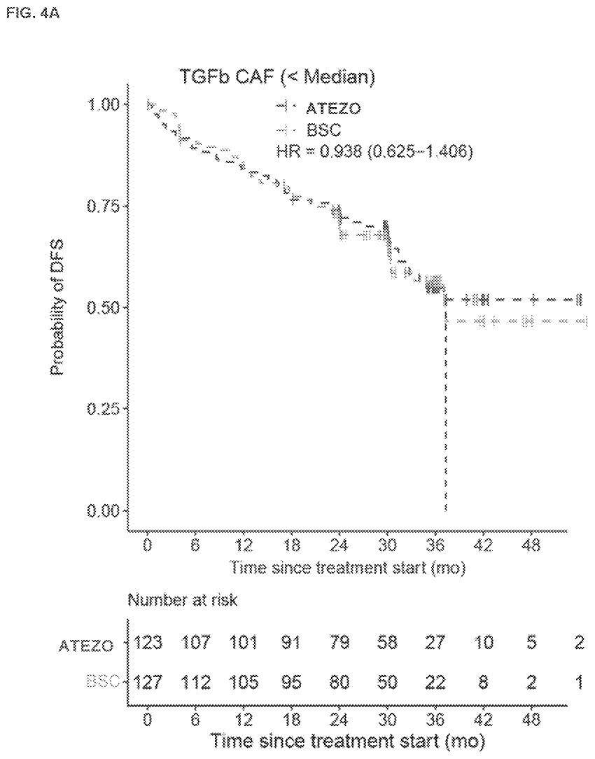

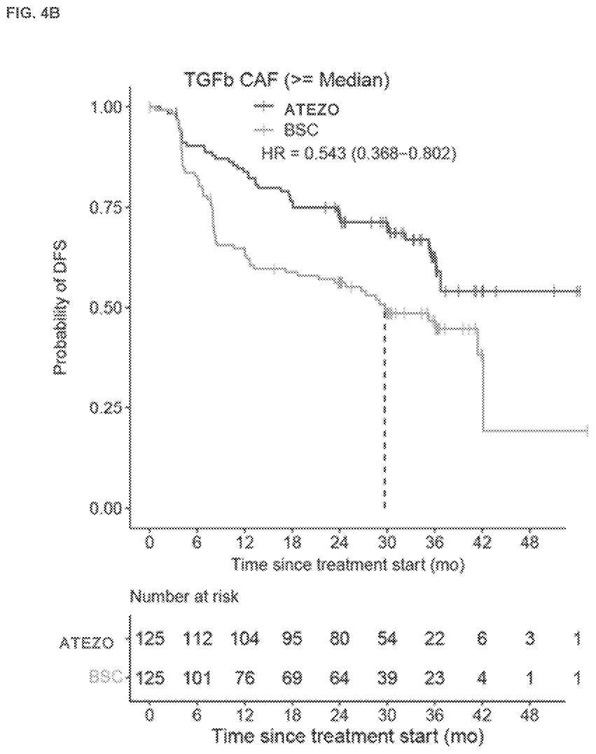

In the TGFβ CAF-low subgroup, the DFS HR for atezolizumab vs BSC was 0.94 (95% CI: 0.63, 1.41); median DFS was NR in the atezolizumab arm and was 37.3 months in the BSC arm (FIG. 4A). In the TGFβ CAF-high subgroup, the DFS HR was 0.54 (95% CI: 0.37, 0.80); median DFS was NR in the atezolizumab arm compared with 29.7 months in the BSC arm (FIG. 4B).

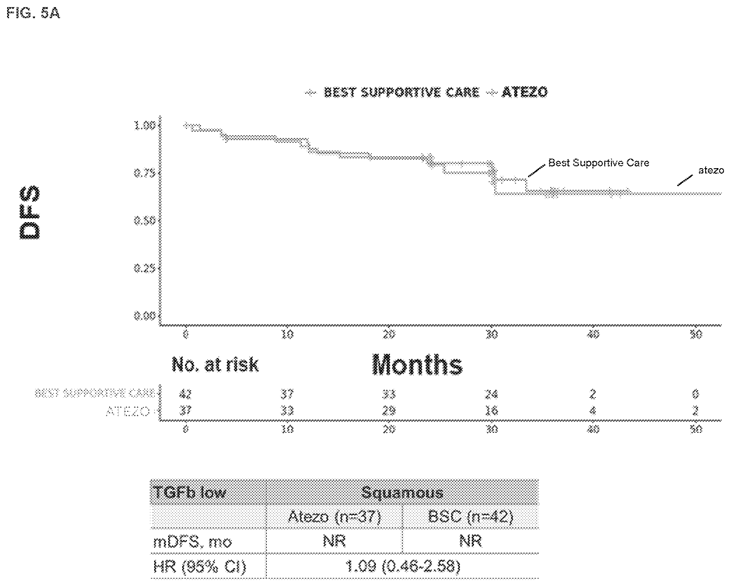

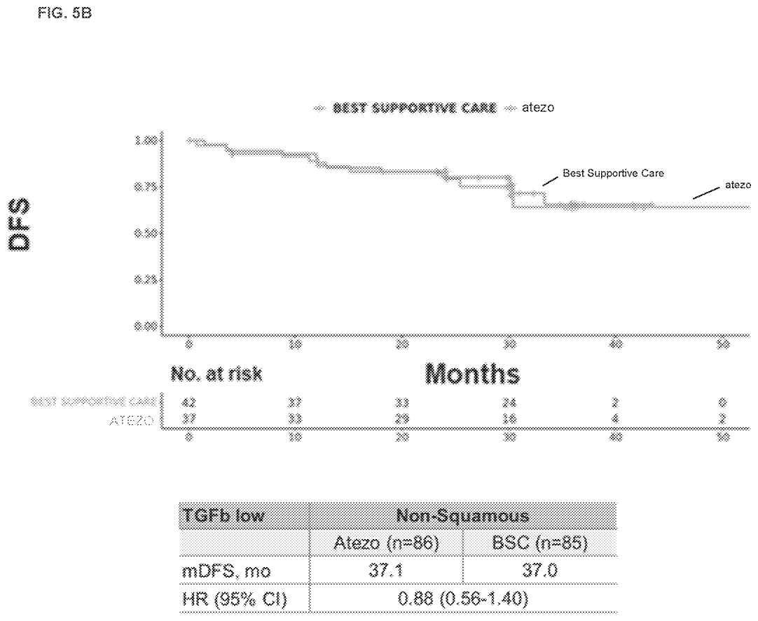

To evaluate the effects of histology within TGFβ CAF subgroups, DFS was examined by squamous or non-squamous histology in the TGFβ CAF-low and -high subgroups. Within the TGFβ CAF-low subgroup, the DFS HR was 1.09 (95% CI: 0.46, 2.58), with the median DFS NR in both arms, in those with squamous histology (FIG. 5A); in those with non-squamous histology, the DFS HR was 0.88 (95% CI: 0.56, 1.40), with a median DFS of 37.1 and 37.0 months in the atezolizumab and BSC arms, respectively (FIG. 5B). Within the TGFβ CAF-high subgroup, the DFS HR was 0.48 (95% CI: 0.26, 0.89), with median DFS NR in the atezolizumab arm and 41.4 months in the BSC arm, in those with squamous histology (FIG. 5C); in those with non-squamous histology, the DFS HR was 0.60 (95% CI: 0.36, 0.99), with a median DFS of 36.1 months in the atezolizumab arm and 24.7 months in the BSC arm (FIG. 5D).

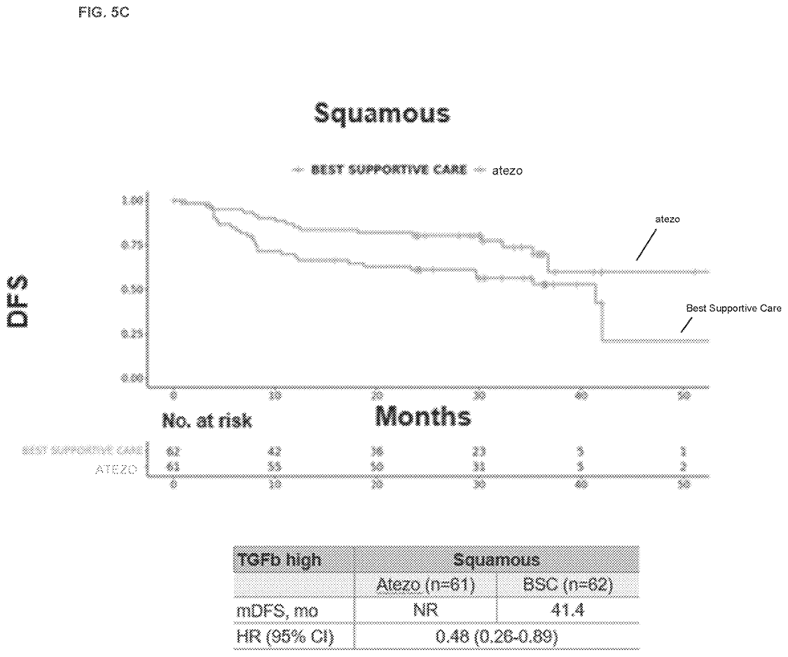

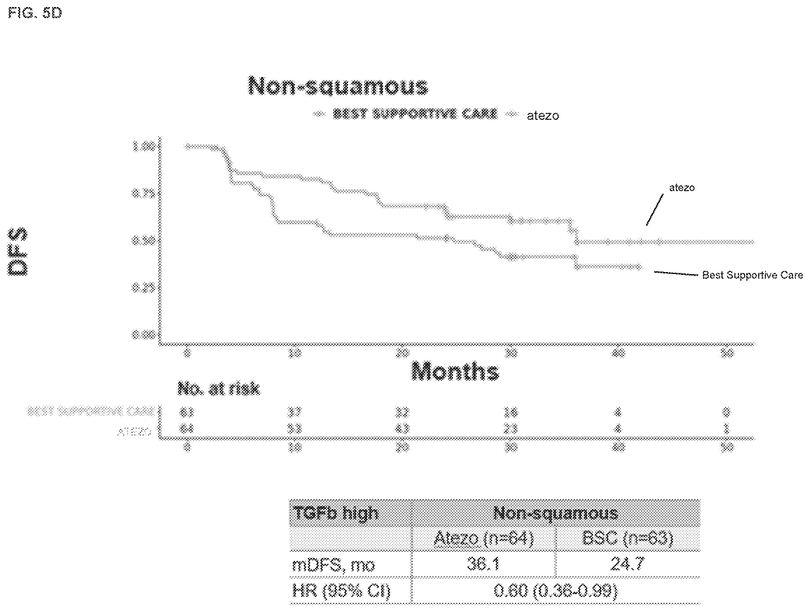

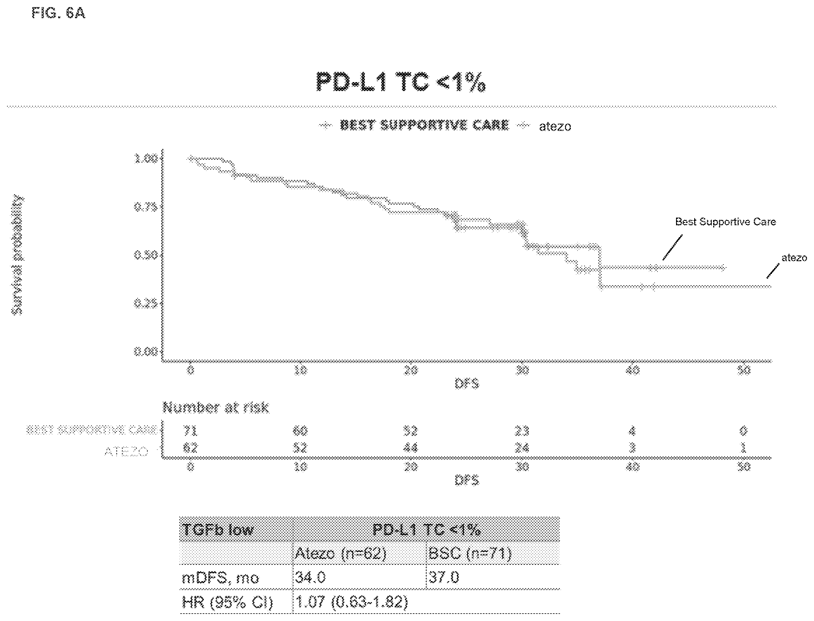

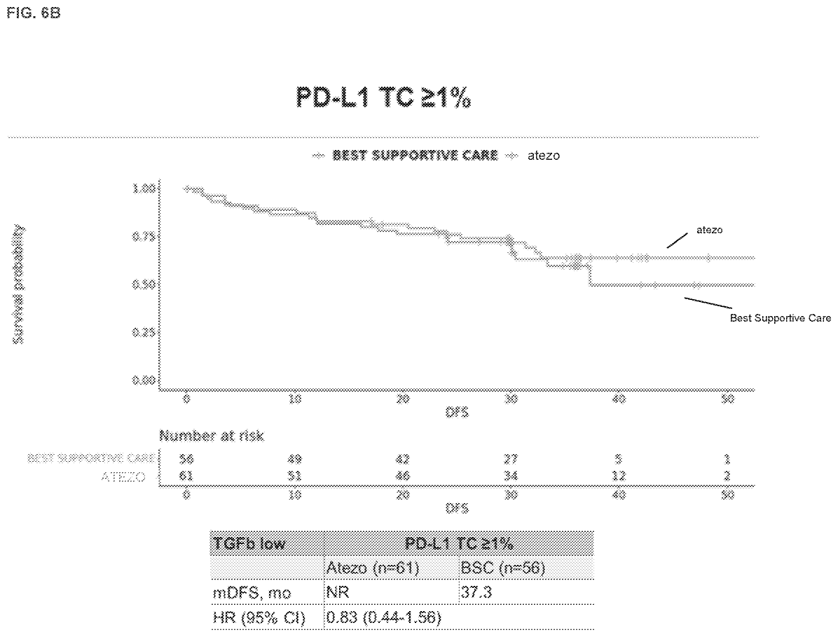

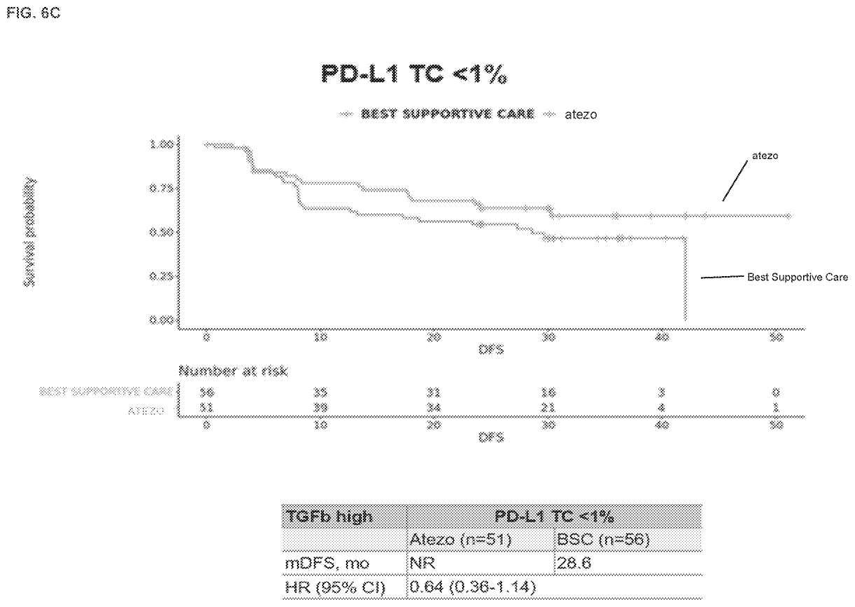

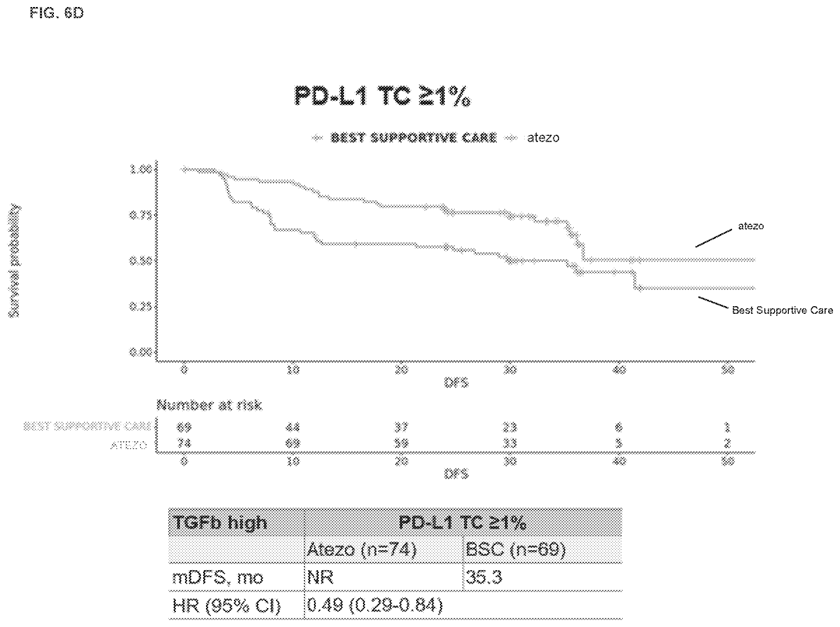

To evaluate the effects of PD-L1 expression within TGFβ CAF subgroups, DFS was examined by PD-L1-negative (TC<1% by SP263) or PD-L1-positive (TC ≥1% by SP263) expression level in the TGFβ CAF-low and -high subgroups. Within the TGFβ CAF-low subgroup, the DFS HR was 1.07 (95% CI: 0.63-1.82), with a median DFS of 34.0 months in the atezolizumab arm and 37.0 months in the BSC arm, in those with PD-L1-negative tumors (FIG. 6A); in those with PD-L1-positive tumors, the DFS HR was 0.83 (95% CI: 0.44-1.56), with the median DFS NR in the atezolizumab arm and 37.3 months in the BSC arm (FIG. 6B). Within the TGFβ CAF-high subgroup, the DFS HR was 0.64 (95% CI: 0.36, 1.14), with median DFS NR in the atezolizumab arm and 28.6 months in the BSC arm, in those with PD-L1-negative tumors (FIG. 6C); in those with PD-L1-positive tumors, the DFS HR was 0.49 (95% CI: 0.29, 0.84), with median DFS NR in the atezolizumab arm and 35.3 months in the BSC arm (FIG. 6D).

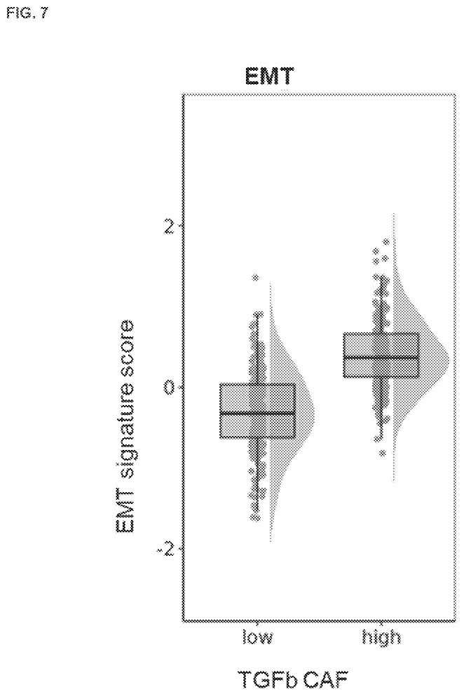

Next, the transcriptomes were compared between the TGFβ CAF subgroups and tumors from the TGFβ CAF-high subgroup were found to have significantly elevated epithelial to mesenchymal transition (EMT) compared with tumors from the TGFβ CAF-low subgroup (FIG. 7).

### Discussion

Using an unbiased approach to bulk tumor RNA-seq, the TGFβ CAF gene signature was identified as a potentially predictive marker of DFS outcome for atezolizumab compared with BSC in individuals with completely resected stage IB-IIIA NSCLC. Median DFS was numerically shorter in the TGFβ CAF-high compared with the TGFβ CAF-low subgroup, suggesting that high TGFβ CAF gene signature expression may be a poor prognostic factor for DFS. Analyses between treatment arms demonstrated that high TGFβ CAF may be predictive of a DFS improvement with atezolizumab compared with BSC, and this relationship appeared consistent regardless of histology or PD-L1 expression level.

TGFβ is a growth factor that drives fibroblast activation into CAFs (Rønnov-Jessen L, Petersen OW. Lab Invest 1993; 68 (6): 696-707). CAFs are one of the most common types of stromal cells found in the tumor microenvironment of solid tumors and have been shown to promote tumor cell growth and metastases (Xing F. Frontiers in Bioscience 2010; 15 (1): 166; Calon A et al., Semin Cancer Biol 2014; 25:15-22). Mouse models have identified leucine-rich repeat containing 15 (LRRC15) protein as a specific marker of TGFβ CAF, and RNA-seq of human samples has confirmed LRRC15+ CAF expression across tumor types, including lung, ovarian, colon, renal, esophageal, stomach, bladder, pancreatic, breast, and head and neck cancers (Krishnamurty A T et al., Nature 2022; 611 (7934): 148-154; Dominguez C X et al., Cancer Discovery 2020; 10 (2): 232-253; Grout J A et al., Cancer Discov 2022; 12 (11): 2606-2625).

Previous analyses of atezolizumab-treated individuals in the metastatic setting (including samples from metastatic urothelial carcinoma, renal cell carcinoma, head and neck cancer, and NSCLC) found that higher LRRC15+ CAF expression was associated with worse overall survival outcomes (Dominguez C X et al., Cancer Discovery 2020; 10 (2): 232-253). The current DFS data are therefore supportive of the previous literature suggesting that a high level of the TGFβ CAF gene signature, or its surrogate marker LRRC15+ CAF, may be a poor prognostic factor for clinical outcomes. In tumors from the TGFβ CAF-high subgroup, enhanced EMT signals were observed compared with those from the TGFβ CAF-low subgroup. EMT in early-stage cancers is associated with invasive properties and metastases (Thiery J. Nature; 2002:442-454). Therefore, it is possible that high EMT signals in the TGFβ CAF-high tumors may lead to a higher propensity for early metastasis compared with TGFβ CAF-low tumors, which may explain the poor prognosis of the TGFβ CAF-high subgroup in the BSC arm. Alternatively, the enrichment of EMT could be a consequence of the higher fibroblast content. Future analyses are required to elucidate the relationship between EMT signals, TGFβ CAF gene signature expression, and clinical prognosis.

While previous literature was limited to comparisons among individuals receiving atezolizumab in the metastatic setting (Dominguez C X et al., Cancer Discovery 2020; 10 (2): 232-253), the current analysis examined outcomes between treatment arms, in the context of early-stage cancer. Although high TGFβ CAF gene signature expression was a poor prognostic feature in the BSC arm, this effect was rescued by atezolizumab, suggesting that high TGFβ CAF gene signature expression may also be a predictive marker of DFS improvement with atezolizumab compared with BSC.

Baseline characteristics and the DFS treatment effect in the RNA-seq BEP were similar to that of the ITT population, including for PD-L1-negative and PD-L1-positive subgroups, suggesting that the RNA-seq BEP was representative of the ITT. Despite a greater prevalence of individuals with squamous disease in the TGFβ CAF-high compared with the TGFβ CAF-low subgroup, histology did not seem to impact the predictive value of the TGFβ CAF gene signature on DFS outcome. In individuals with low TGFβ CAF gene signature expression, a similar DFS outcome was seen for the atezolizumab and BSC arms for both squamous and non-squamous subgroups, while a DFS improvement was seen with atezolizumab compared with BSC for both the squamous and non-squamous TGFβ CAF-high subgroups.

Similarly, despite a numerically higher prevalence of individuals with PD-L1-negative tumors in the TGFβ CAF-low vs high subgroup, PD-L1 expression did not seem to impact the predictive value of TGFβ CAF gene signature expression on DFS outcome. For individuals with high TGFβ CAF gene signature expression, a DFS improvement was seen with atezolizumab regardless of PD-L1 expression level. These data suggest that individuals who are PD-L1-negative but TGFβ CAF-high may still derive benefit from atezolizumab. Therefore, understanding TGFβ CAF gene signature status in combination with PD-L1 expression may be an important tool to identify the individuals that are most likely to respond to atezolizumab.

Samples in this study were collected prior to chemotherapy or atezolizumab treatment. This analysis supports the evaluation of TGFβ CAF gene signature expression levels from resected tumor tissues to help identify which individuals may derive the greatest benefit from adjuvant atezolizumab. It is unknown if samples collected after treatment, as could be the case in the metastatic setting, would offer the same prognostic or predictive value.

In conclusion, this analysis is the first to show an association between TGFβ CAF and clinical outcomes with an immune checkpoint inhibitor in individuals with early-stage NSCLC. Data from the BSC arm showed that high TGFβ CAF gene signature expression may be a poor prognostic factor for DFS. These data suggest that individuals with enrichment of the TGFβ CAF gene signature may derive benefit from adjuvant atezolizumab compared with BSC after surgical resection, regardless of histology or PD-L1 expression level. In addition, these data suggest that LRRC15 may serve as a single-gene biomarker for individuals who may derive benefit from adjuvant atezolizumab compared with BSC after surgical resection, regardless of histology or PD-L1 expression level.

### Methods

### Study Design

IMpower010 is a randomized, multicenter, open-label, Phase III study comparing adjuvant atezolizumab or BSC after cisplatin-based doublet chemotherapy in individuals with completely resected stage IB-IIIA NSCLC (Felip E et al., Lancet, Vol 398, 1344-1357). Individuals must have had completely resected stage IB-IIIA NSCLC per International Union Against Cancer/American Joint Committee on Cancer v7, an Eastern Cooperative Oncology Group (ECOG) Performance Status of 0 or 1, prior lobectomy or pneumonectomy, and tumor tissue available for PD-L1 analysis. Following 1-4 cycles of cisplatin plus pemetrexed, gemcitabine, docetaxel, or vinorelbine, eligible individuals were randomized 1:1 to receive atezolizumab (1200 mg IV) every 3 weeks for 16 cycles or <16 cycles of BSC; crossover was not allowed. Stratification factors included sex, stage, histology, and PD-L1 status per the SP142 assay. The primary endpoint was DFS tested hierarchically in individuals with PD-L1-positive (TC ≥1% by SP263) stage II-IIIA tumors, all randomized individuals with stage II-IIIA tumors, and then all randomized individuals with stage IB-IIIA tumors (intention-to-treat [ITT] population).

### RNA-Sequencing

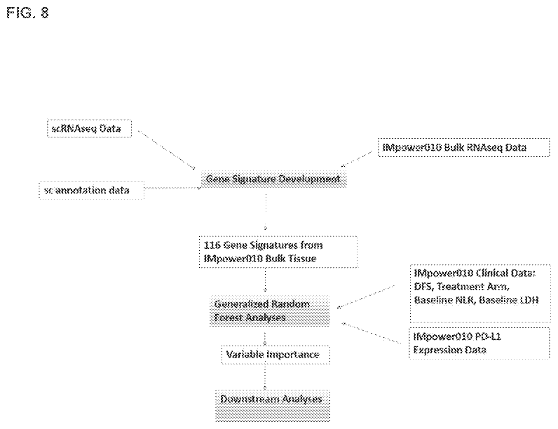

Bulk tumor RNA-seq was performed on baseline tumor resections using an unbiased examination with an input of 116 gene signatures related to cell type and cell state, as well as other relevant biomarkers (FIG. 8).

RNA-seq libraries were created using the Illumina TRUSEQ® Stranded Total RNA method, and ribosomal RNA was preferentially removed prior to library generation using the RIBO-ZERO® Magnetic Gold kit (LAB_13_3188). Libraries were sequenced with the Illumina sequencing-by-synthesis platform on the NOVASEQ® 6000 using a sequencing protocol of 50 bp paired-end strategy and a total read depth of 80 million reads per sample.

The predictive effects of atezolizumab compared with BSC treatment were analyzed using generalized random forests methods (Cui Y et al., Journal of the Royal Statistical Society Series B: Statistical Methodology: 179-211; Athey S et al., The Annals of Statistics. 47. 2019:1148-1178), and the methods were implemented by the R package “grf” (Tibshirani J et al, grf: Generalized Random Forests. R package version 2.2.0). Relevant features were determined based on variable importance, defined as the weighted sum of the frequency that a feature was selected in the forest.

### Statistics

In the exploratory subgroup analyses, median DFS was estimated using Kaplan-Meier curves. Unstratified hazard ratios (HR) were estimated by Cox regressions.

### Example 2: Impower010: Exploratory Analysis of Disease-Free Survival and Overall Survival with Atezolizumab or Best Supportive Care by TGFβ Cancer-Associated Fibroblast Gene Signature Status in Individuals with Early Non-Small Cell Lung Cancer

### Introduction

In this study, bulk tumor RNA-seq from the IMpower010 study at the time of resection was used to investigate the molecular features associated with PD-L1 tumor cell expression in addition to potentially predictive features of adjuvant atezolizumab benefit compared to best supportive care (BSC) in individuals with early NSCLC.

### Results

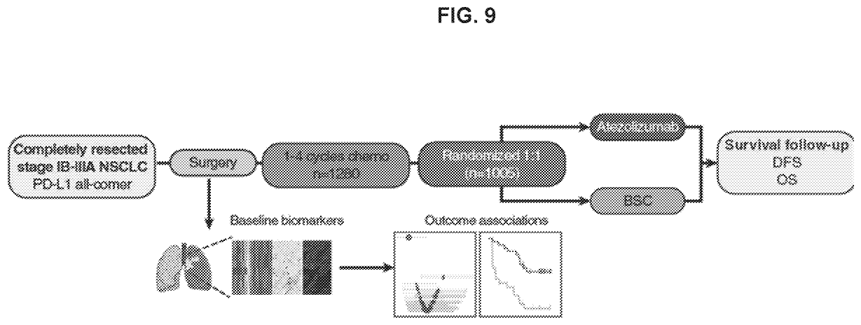

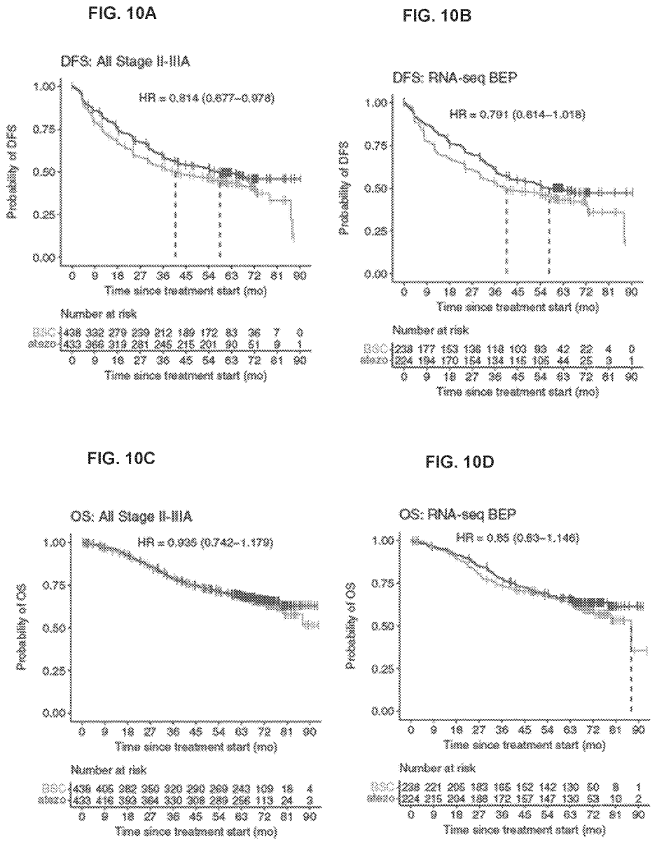

Of the 1005 patients randomized to receive BSC or atezolizumab following surgery and chemotherapy (i.e., the intention-to-treat (ITT) population), 462 of these patients had tissue available for RNA-seq and constituted the RNA-seq biomarker evaluable population (BEP) (FIG. 9). Baseline characteristics of the RNA-seq BEP were similar (i.e., <10% different) to those of the ITT population (Table 4). Outcomes for disease-free survival (DFS) and overall survival (OS) were similar between Stage II-IIIA patients compared to the RNA-seq BEP based on data from the 5-year DFS and OS at the clinical cutoff date of Jan. 26, 2024. Outcomes for DFS and OS were generally similar between all Stage II-IIIA patients compared to the RNA-seq BEP based on data from the 5-year DFS and OS (FIGS. 10A-10D).

TABLE 4
Baseline demographics and characteristics

| | Stage II-IIIA | RNASeq | High TGFB CAF | Low TGFB CAF |
|---|---|---|---|---|
| | (n = 871) | BEP (n = 462) | (n = 231) | (n = 231) |
| Median age (range), y | 62 (26-84) | 62 (26-84) | 61 (34-78) | 62 (26-84) |
| Male, n (%) | 581 (66.7) | 320 (69.3%) | 167 (72.3) | 153 (66.2) |
| ECOG PS, n (%) | | | | |
| 0 | 488 (56.0) | 251 (54.3) | 126 (54.5) | 125 (54.1) |
| 1 | 380 (43.6) | 209 (45.2) | 104 (45.0) | 105 (45.5) |
| 2 | 3 (0.3) | 2 (0.4) | 1 (0.4) | 1 (0.4) |
| Smoking history, n (%) | | | | |
| Current | 135 (15.5) | 95 (20.6) | 48 (20.8) | 47 (20.3) |
| Never | 192 (22.0) | 85 (18.4) | 34 (14.7) | 51 (22.1) |
| Previous | 544 (62.5) | 282 (61) | 149 (64.5) | 133 (57.6) |
| Histology, n (%) | | | | |
| Non-squamous | 581 (66.7) | 274 (59.3) | 111 (48.1) | 163 (70.6) |
| Squamous | 290 (33.3) | 188 (40.7) | 120 (51.9) | 68 (29.4) |
| PD-L1 expression by | | | | |
| SP263, n (%) | | | | |
| TC <1% | 379 (43.5) | 221 (47.8) | 100 (43.4) | 121 (52.4) |
| TC 1-49% | 245 (28.1) | 128 (27.7) | 71 (30.7) | 57 (24.7) |
| TC ≥50% | 225 (25.8) | 111 (24.0) | 58 (25.1) | 53 (22.9) |
| NA | 22 (2.5) | 2 (0.4) | 2 (0.9) | 0 (0) |
| Stage, n (%) | | | | |
| Stage IIA | 292 (33.5) | 138 (29.9) | 82 (35.5) | 56 (24.2) |
| Stage IIB | 173 (19.9) | 99 (21.4) | 46 (19.9) | 53 (22.9) |
| Stage IIIA | 406 (46.6) | 225 (48.7) | 103 (44.6) | 122 (52.8) |
| EGFR mutant, n (%) | | | | |
| Detected | 108 (12.4) | 38 (8.2) | 23 (10.0) | 15 (6.5) |
| Not detected | 457 (52.5) | 240 (51.9) | 91 (39.4) | 149 (64.5) |
| Unknown | 306 (35.1) | 184 (39.8) | 117 (50.6) | 67 (29.0) |
| ALK mutant, n (%) | | | | |
| Yes | 30 (3.4) | 10 (2.2) | 4 (1.7) | 6 (2.6) |
| No | 501 (57.5) | 246 (53.2) | 106 (45.9) | 140 (60.6) |
| Unknown | 340 (39.0) | 206 (44.6) | 121 (52.4) | 85 (36.8) |

ECOG: Eastern Cooperative Oncology Group (Oken M M et al., Am J Clin Oncol. 1982 December; 5(6): 649-655); IC: tumor-infiltrating cells; IHC: immunohistochemistry; TC: tumor cells.

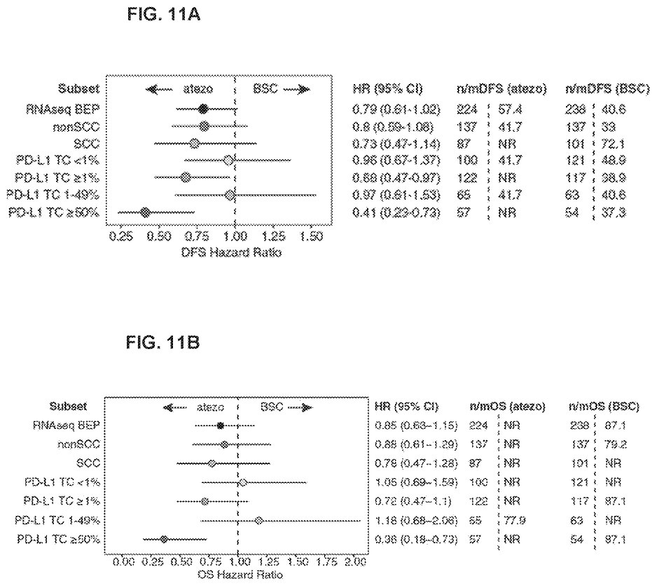

RNA-seq BEP was first characterized based on typical histological and PD-L1 tumor cell (TC) expression subgroups. DFS and OS HRs favored atezolizumab in both squamous and non-squamous histologies (FIGS. 11A-11B). The RNA-seq BEP, like the overall population, had observed DFS and OS benefit for atezolizumab over BSC in both the PD-L1 TC ≥1% and ≥50% expression subgroups, as determined by SP263 detection (FIGS. 11A-11B).

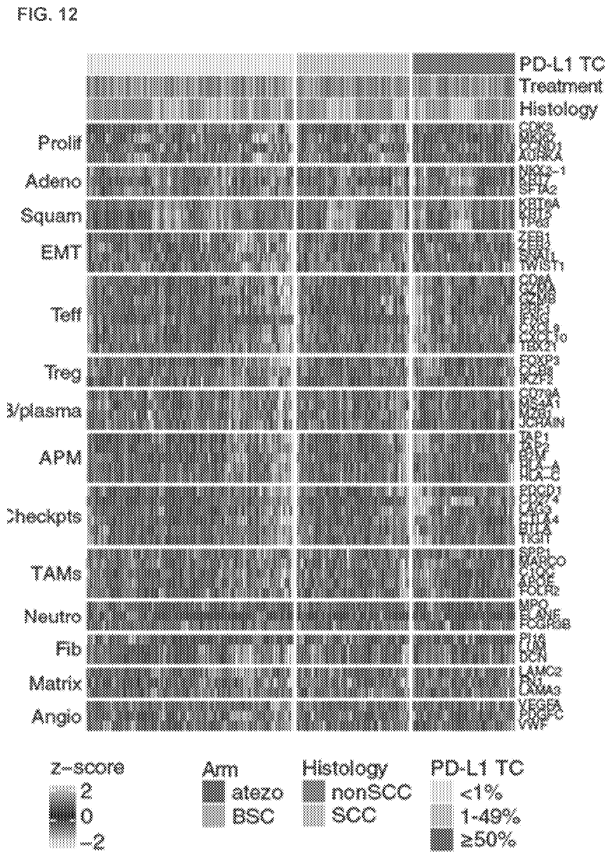

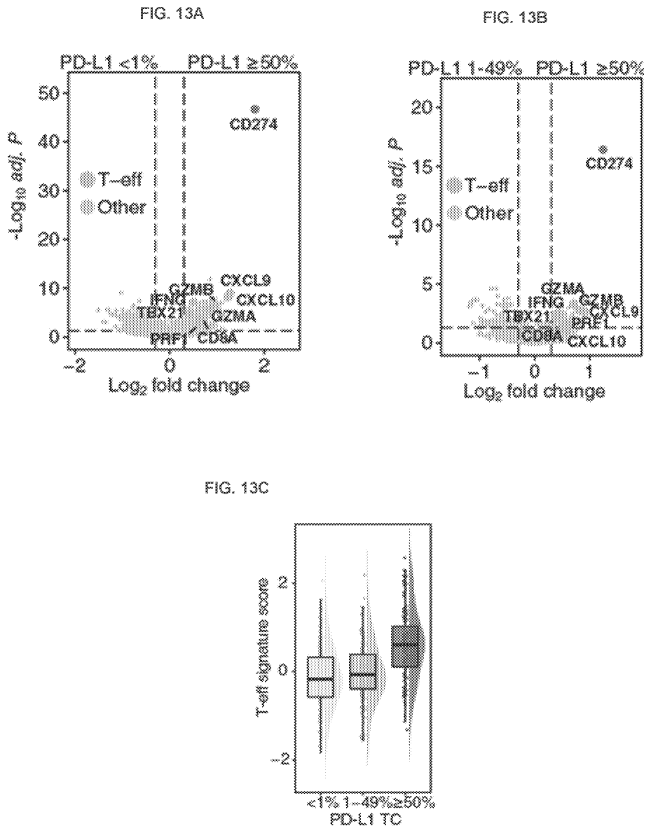

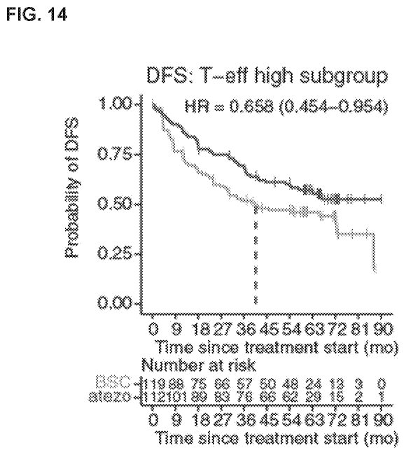

The gene expression correlates of PD-L1 subgroups measured by immunohistochemistry were then examined because of the strong association with clinical benefit and PD-L1 expression in tumor cells. Overall, general measures of immune infiltrate were elevated in tumor samples that had PD-L1 expression of an area covering≥1% of tumor cells (PD-L1 TC>1%) and in tumor samples that had PD-L1 expression of an area covering≥50% of tumor cells (PD-L1 TC ≥50%) (FIG. 12). Further, comparisons of differentially expressed genes between PD-L1 TC samples showed a marked upregulation of individual T-effector signature genes (e.g., CD8A, CXCL9, CXCL10, GZMA, GZMB, IFNG, PRF1, and TBX21) and the T-effector signature score in PD-L1 TC ≥50% samples compared to both PD-L1-negative and PD-L1 TC 1-49% samples (FIGS. 13A-13C). When dichotomizing patients as T-effector high or T-effector low groups based on median expression of the signature score irrespective of PD-L1 status, atezolizumab benefit as compared to BSC was similar to the benefit of PD-L1 TC ≥1% (FIG. 14). Overall, these data suggest that T-effector signals are highly associated with high PD-L1 expression measured by IHC and are not an independent predictor of adjuvant PD-L1 blockade benefit in early NSCLC.

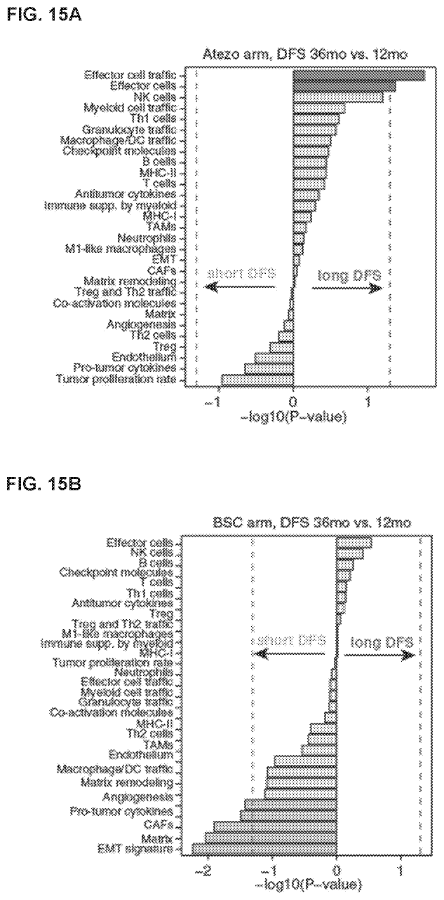

Differentially expressed signatures that define pan-cancer microenvironment subtypes were examined to uncover additional features that may be predictive of atezolizumab vs. BSC benefit in early NSCLC. Gene signatures associated with effector cell traffic and/or effector cells were enriched in long DFS (>36 months) compared to short DFS (<12 months) in the atezolizumab arm, which was consistent with the strong association with PD-L1 high tumors (FIG. 15A). The same comparison within the BSC arm demonstrated an enrichment for epithelial to mesenchymal transition, matrix deposition, and cancer-associated fibroblasts (CAFs) in short DFS (FIG. 15B). These analyses suggest that matrix and/or CAF-rich tumors may confer shorter DFS and poor outcomes in the absence of adjuvant PD-L1 blockade.

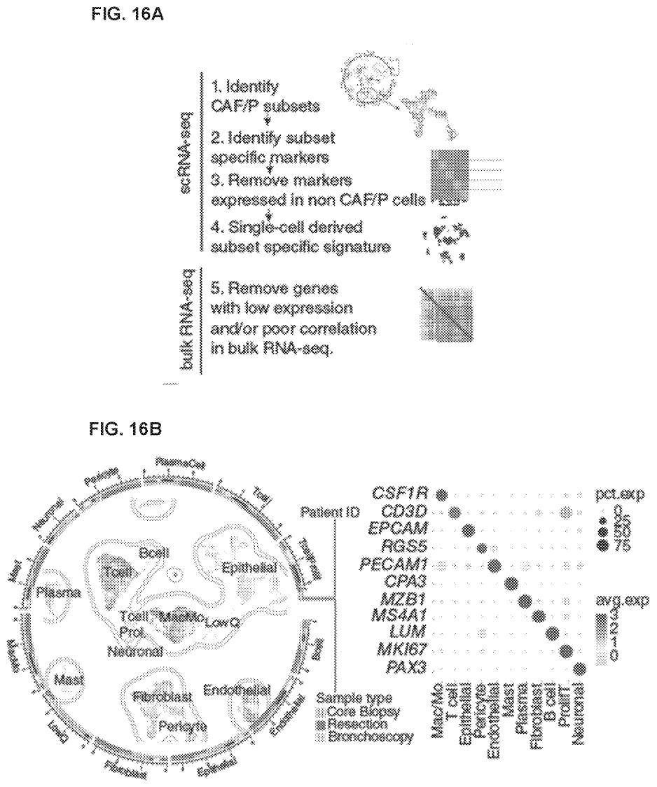

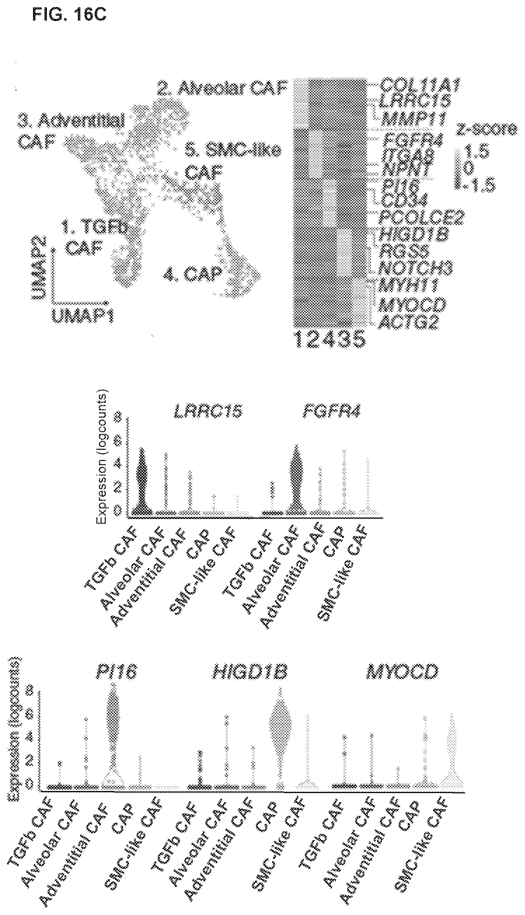

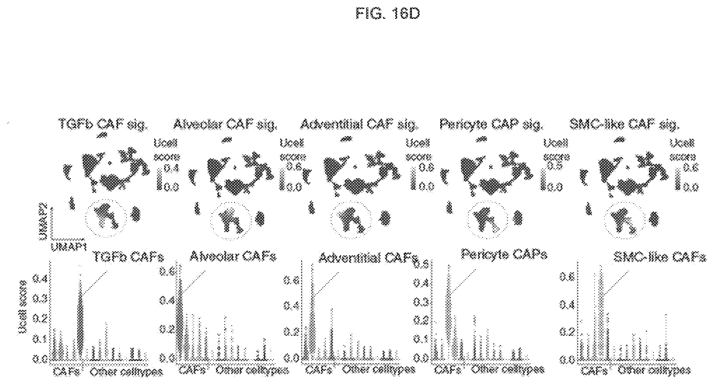

Given the association of stromal signals with poor outcomes in the BSC arm, the CAF compartment of NSCLC was then characterized by analyzing NSCLC single-cell RNA-seq data to define robust gene signatures for the stromal compartment (FIG. 16A). Major cell type groups were well represented across patients and sample collection type, and each group was distinguishable by established cell type-specific markers that were not measured in any other cell type, including markers corresponding to adventitial CAFs, alveolar CAFs, smooth muscle cell-like CAFs, TGFβ CAFs, and cancer-associated pericytes (CAPs) (FIGS. 16B-16D). The genes comprising the TGFβ CAF gene signature included: LRRC15, FAP, COL11A1, COL10A1, FNDC1, GREM1, ITGA11, WISP1, PLPP4, PODNL1, TWIST1, COL8A2, SUGCT, NOX4, ZNF469, TENM3, P4HA3, ADAMTS12 and CPZ.

To identify additional biological features for DFS benefit with atezolizumab compared to BSC that may be independent of PD-L1 TC expression or T-effector expression, a generalized random forests method for analyzing bulk tumor RNA-seq data was implemented. The model had an input of 72 curated gene signatures related to cell type and cell state, including tumor microenvironment (TME) subtype, Hallmark, B cell subset gene signatures, T-effector signature, and newly derived CAF or CAP signatures. Also included were baseline lactate dehydrogenase levels, baseline neutrophil to lymphocyte ratio, and PD-L1 TC IHC scores to account for prognostic factors.

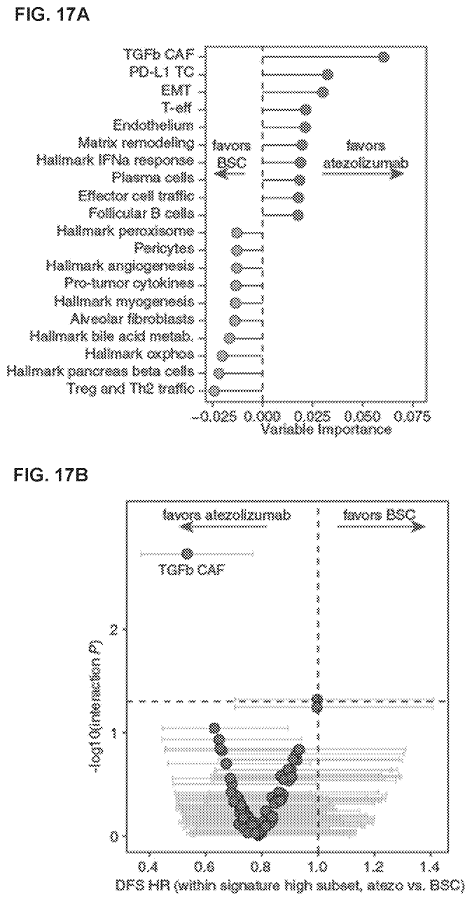

The model identified several candidate features that may be predictive of atezolizumab benefit compared to BSC benefit in adjuvant NSCLC. These analyses identified the TGFβ CAF signature as having the highest variable importance for the model, followed by PD-L1 TC (FIG. 17A), which suggested that tumors with high TGFβ CAF expression may enrich patients that preferentially benefit from atezolizumab as compared to BSC. As an orthogonal approach, the interaction between the treatment arm, each gene signature subset, and DFS as a univariate analysis were also assessed. From this analysis, the interaction test for the TGFβ CAF signature for DFS was significant (P=0.002; FIG. 17B). In sum, these data suggest that TGFβ CAFs are a predictive feature for atezolizumab benefit as compared to BSC.

Next, the TGFβ CAF gene signature expression was assessed in all patients in the RNA-seq BEP. Patients were grouped into TGFβ CAF-low (<median, n=231) or TGFβ CAF-high (≥median, n=231) subgroups. Baseline characteristics were generally well balanced (<10% different) between these two subgroups as shown in Table 4. The prevalence of squamous histology was greater in the TGFβ CAF-high subgroup compared to the TGFβ CAF-low subgroup.

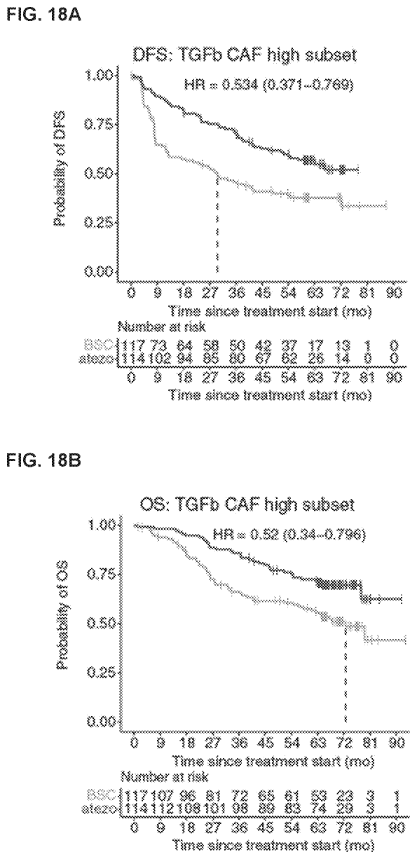

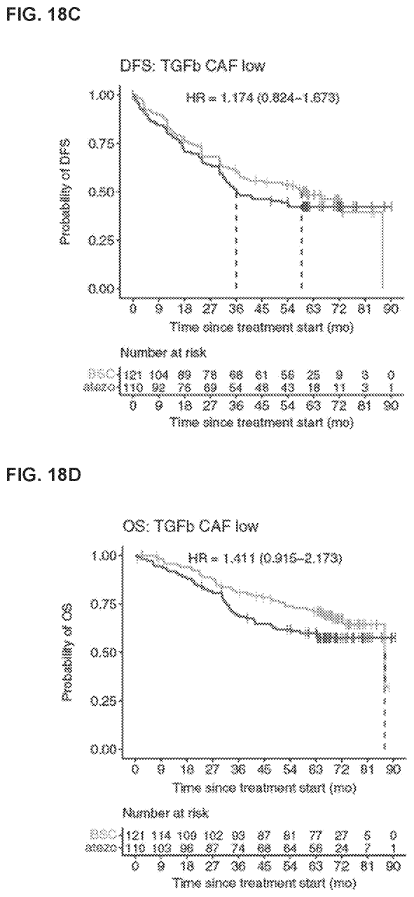

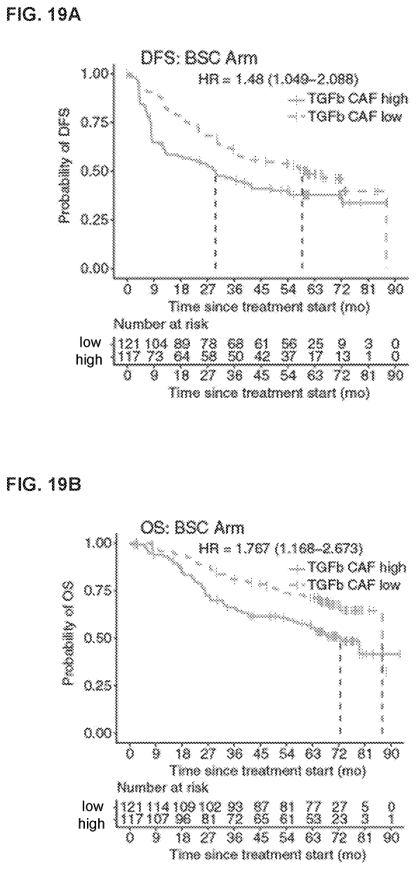

Within the TGFβ CAF-high subset, longer DFS and OS were observed in the atezolizumab arm compared to the BSC arm (DFS HR=0.53 [95% CI: 0.37, 0.77]; OS HR=0.52 [95% CI: 0.34, 0.80]; FIGS. 18A-18B). Conversely, in the TGFβ CAF-low subgroup there was similar DFS and OS in the atezolizumab and BSC arms (DFS HR=1.17 [95% CI: 0.82, 1.67]; OS HR=1.41 [95% CI: 0.92, 2.17]; FIGS. 18C-18D). The TGFβ CAF-high and TGFβ CAF-low subsets within the BSC arm were then examined, since the initial analyses suggested that matrix and CAF-related signals were enriched in BSC patients with short DFS (FIGS. 15A-15B). As expected, the TGFβ CAF-high subset had significantly shorter DFS and OS compared to the TGFβ CAF-low subset in the BSC arm (DFS HR=1.48 [95% CI: 1.05, 2.09]; OS HR=1.77 [95% CI: 1.17, 2.67]; FIGS. 19A-19B). These data suggest that the atezolizumab benefit over BSC observed in the TGFβ CAF-high subgroup is driven by the poor DFS in the BSC arm, which may be overcome with administration of adjuvant atezolizumab.

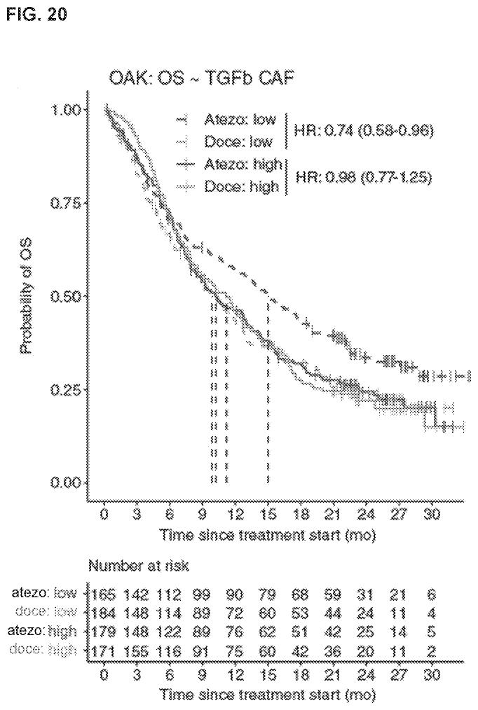

To examine the impact of the TGFβ CAF gene signature expression in an advanced or metastatic NSCLC dataset, OS outcomes were compared for atezolizumab and docetaxel in patients with TGFβ CAF-high and TGFβ CAF-low subgroups from the Phase III OAK study (NCT02008227). In the TGFβ CAF-high subgroup, no benefit was observed with atezolizumab treatment as compared to docetaxel (OS HR for atezolizumab vs. docetaxel=0.74 [95% CI: 0.58, 0.96]; FIG. 20). These results align with metastatic urothelial carcinoma data that showed that the TGFβ CAF gene signature was associated with poor outcomes in the atezolizumab, which suggests that the role of TGFβ CAFs and outcome associations may be distinct in early cancers as compared to metastatic cancers. Such differences may be further affected by treatment differences in adjuvant therapy for early NSCLC as compared to systemic therapy for metastatic NSCLC, in which the primary tumor has been resected prior to systemic therapy in an adjuvant context, in contrast to metastatic NSCLC in which the primary tumor is intact prior to the systemic therapy.

The TGFβ CAF signatures were next evaluated in the early-stage context, in which OS outcomes in TGFβ CAF-high and TGFβ CAF-low subgroups were compared for adjuvant atezolizumab versus observation in patients with muscle-invasive urothelial carcinoma from the Phase III IMvigor010 study (NCT02460331). This analysis focused on the circulating tumor DNA-positive (ctDNA+) subgroup since previous analyses showed that atezolizumab significantly prolonged OS in patients that were ctDNA+ after surgery, as compared to observation.

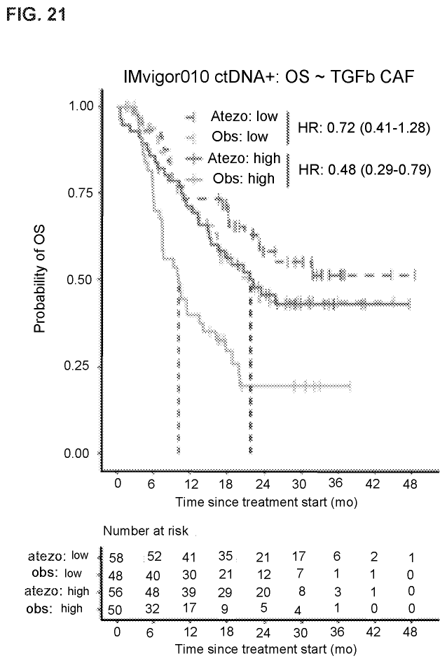

In the ctDNA+ patients, atezolizumab was associated with improved OS as compared to observation in the TGFβ CAF-high subgroup (OS HR for atezolizumab versus observation=0.48 [95% CI: 0.39, 0.79]) but had limited benefit in the TGFβ CAF-low subgroup (OS HR for atezolizumab versus observation=0.72 [95% CI: 0.41, 1.28]; FIG. 21). Like the observed differences in TGFβ CAF subgroups of the IMpower010 study, these differences were driven by the BSC arm having poor outcomes in the TGFβ CAF-high subgroup. Thus, the early stage setting for both urothelial carcinoma and NSCLC and high expression of the TGFβ CAF gene signature may identify a subgroup of patients with poor outcomes in the absence of adjuvant atezolizumab.

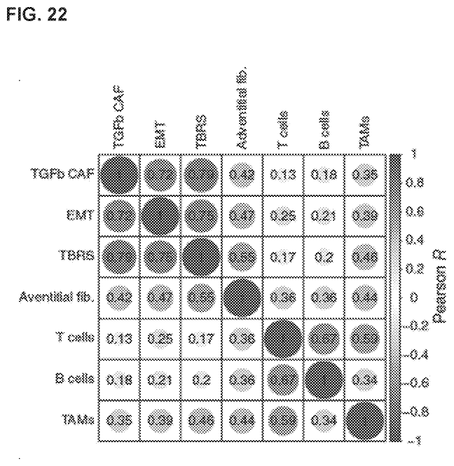

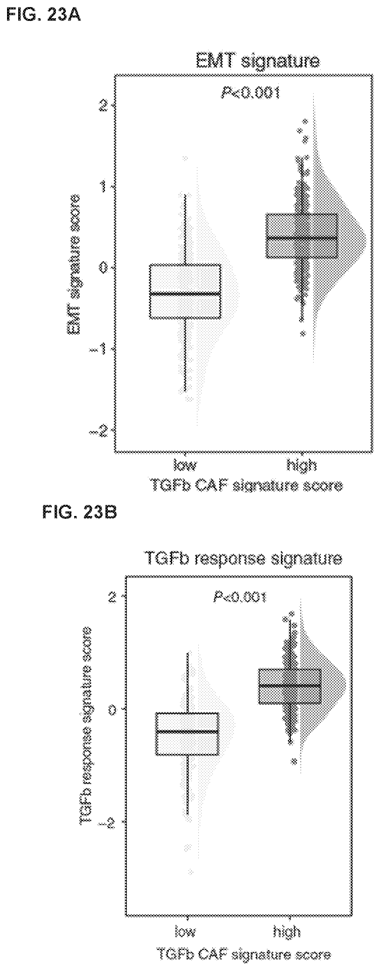

Next, the specific enrichment of TGFβ CAFs compared to other features were evaluated to further understand the association of the TGFβ CAF gene signature and clinical outcomes. To this end, the TGFβ CAF gene signature was compared to other relevant cell types and signatures (FIG. 22). The TGFβ CAF gene signature was highly correlated to a TGFβ response signature (TBRS; R=0.79), which was experimentally defined to recapitulate active TGFβ signaling, despite no overlapping genes. The TGFβ CAF signature was also highly correlated to an epithelial to mesenchymal transition (EMT) signature (R=0.72), which is consistent with the role of TGFβ in regulating EMT (FIG. 22). Expression of the EMT and TBRS gene signatures were significantly elevated in tumors from the TGFβ CAF-high subgroup as compared to tumors from the TGFβ CAF-low subgroup (P=<0.001; FIGS. 23A-23B). In total, these results suggest that TGFβ CAFs are associated with general TGFβ-related signaling and EMT. There was a modest correlation (R=0.42) between TGFβ CAFs and a gene signature corresponding to adventitial fibroblasts (e.g., a universal fibroblast phenotype in all tissues), which suggested that the TGFβ CAF enrichment was not solely due to an increase in total fibroblasts. Further, the TGFβ CAF gene signature was weakly correlated with tumor-associated macrophage (R=0.35), B cell (R=0.18), and T cell (R=0.13) gene signatures (FIG. 22).

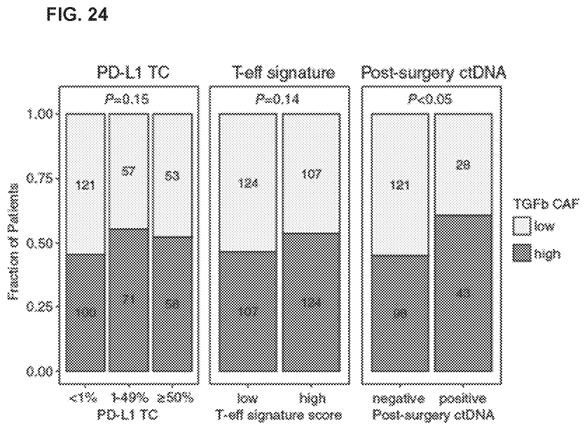

Comparison of other features associated with atezolizumab benefit revealed that the TGFβ CAF gene signature score was similar across PD-L1 expression levels and T-effector gene signature subgroups (FIG. 24). Together, these data suggest the association of the TGFβ CAF gene signature with the DFS and OS outcomes in the IMpower010 clinical study are independent of traditional biomarkers of immune checkpoint inhibitor benefit.

Next, to understand whether TGFβ CAF-high tumors have a higher propensity for early metastatic dissemination, the relative proportion of post-resection, pre-chemotherapy ctDNA+ patients as a marker of minimal residual disease were compared in the TGFβ CAF subgroups. TGFβ CAF-high tumors were significantly more likely to be ctDNA+ post-surgery and prior to chemotherapy, which suggests residual disease and/or undetected micrometastases (FIG. 24).

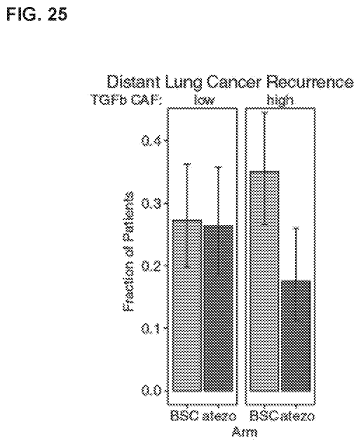

The metastatic propensity was further examined by evaluating the patterns of recurrence in each treatment arm and TGFβ CAF subgroup. Similar rates of distant lung cancer recurrence were observed in the TGFβ CAF-low subgroup in the BSC arm (27.3% [95% CI: 19.8, 36.3%]) and the atezolizumab arm (26.4% [95% CI: 18.6, 35.8%]), as shown in FIG. 25. Conversely, in the TGFβ CAF-high subgroup, there was a higher fraction of distant lung cancer recurrence in the BSC arm (35.0% [95% CI: 26.6, 44.5%]) compared to the atezolizumab arm (17.5% [95% CI: 11.3, 26.0%]), as shown in FIG. 25. These results, together with the observed increase of ctDNA positivity following surgery, suggest that TGFβ CAF-high tumors may be more likely to recur at a distant site in the absence of adjuvant atezolizumab.

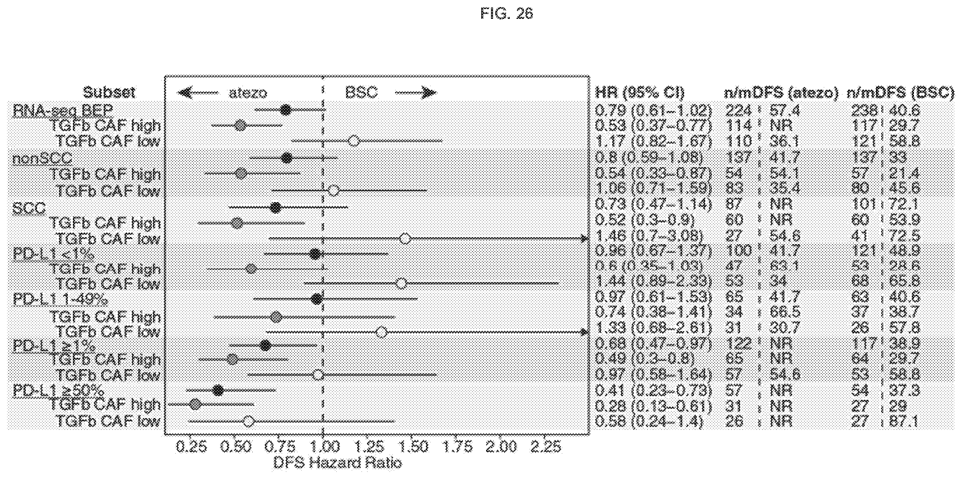

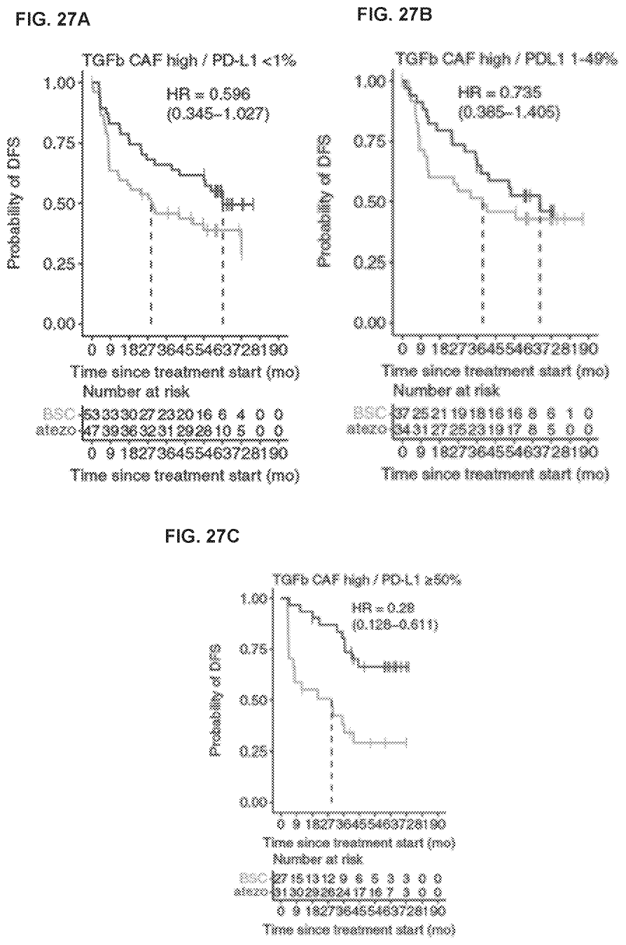

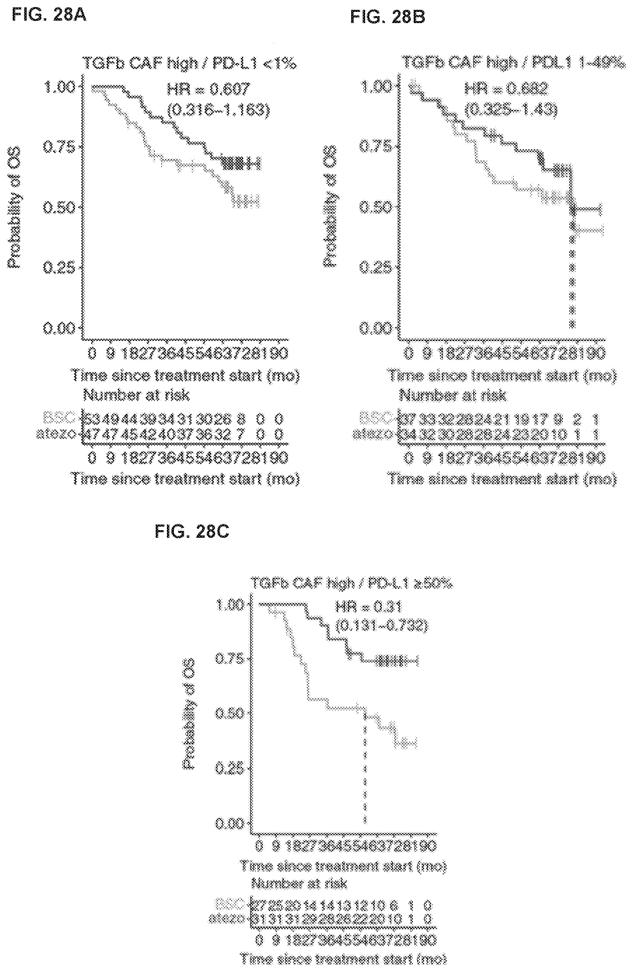

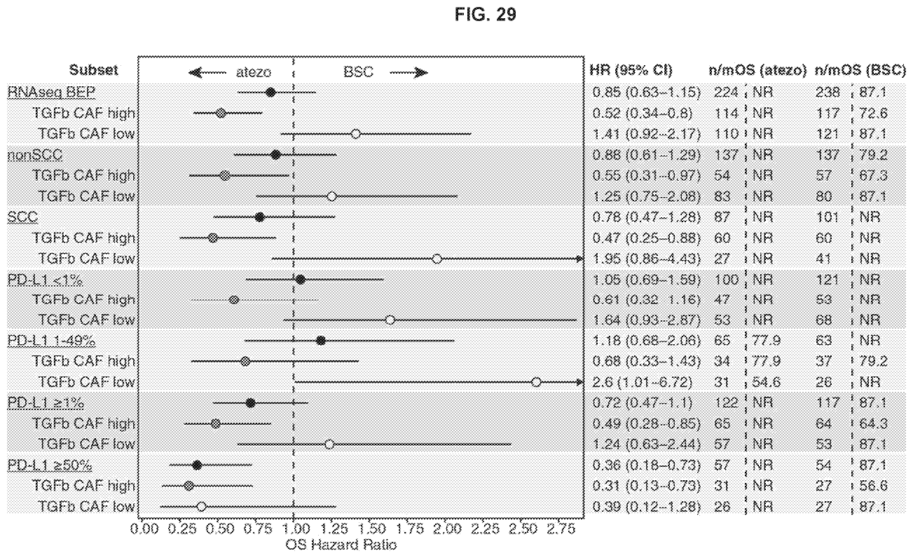

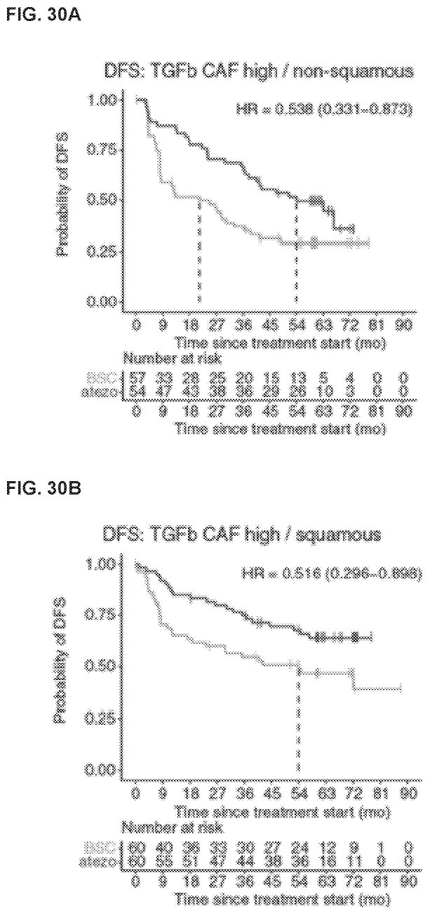

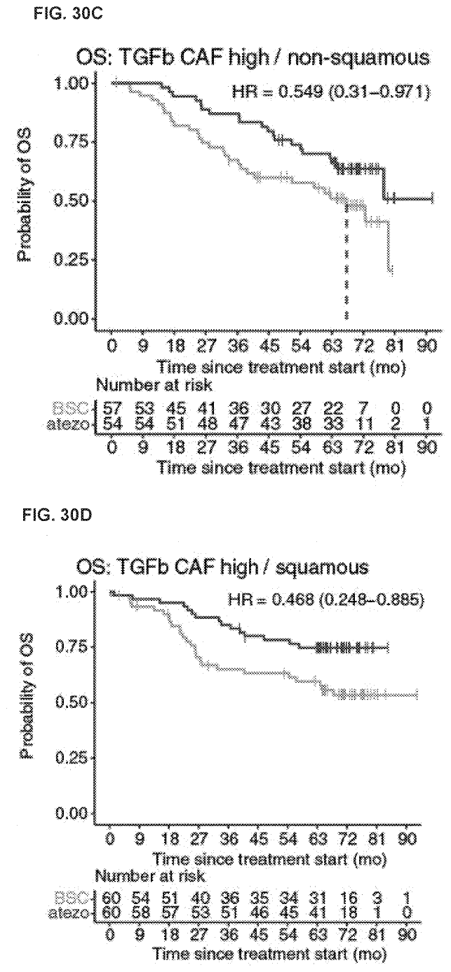

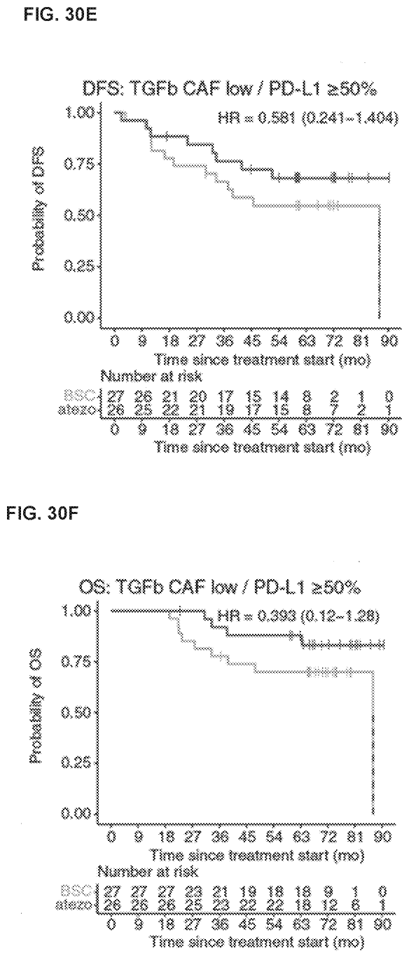

To further understand if the atezolizumab benefit observed in the TGFβ CAF-high tumors was independent of histology and PD-L1 status, the DFS and OS outcomes were compared in the TGFβ CAF-high and TGFβ CAF-low tumor subgroups for atezolizumab treatment and BSC. Irrespective of PD-L1 status and histology, TGFβ CAF-high tumors, but not TGFβ CAF-low tumors, demonstrated longer DFS and OS with atezolizumab compared to BSC (FIGS. 26-30). Notably, even in PD-L1-negative and tumors with PD-L1 expression of 1-49%, there was minimal overall atezolizumab benefit observed in the overall population, the TGFβ CAF-high tumors, but not the TGFβ CAF-low tumors, showed DFS and OS benefit (FIGS. 27A-27C and 28A-28C). Interestingly, the TGFβ CAF-low tumors showed atezolizumab benefit in tumors that had PD-L1 expression>50%, suggesting that high tumor expression of PD-L1 benefits from atezolizumab treatment independently from TGFβ CAF expression (FIGS. 26, 27C, and 28C). Thus, TGFβ CAF gene signature expression may be a predictive biomarker of benefit from atezolizumab treatment compared to BSC that is independent of PD-L1 expression and histology.

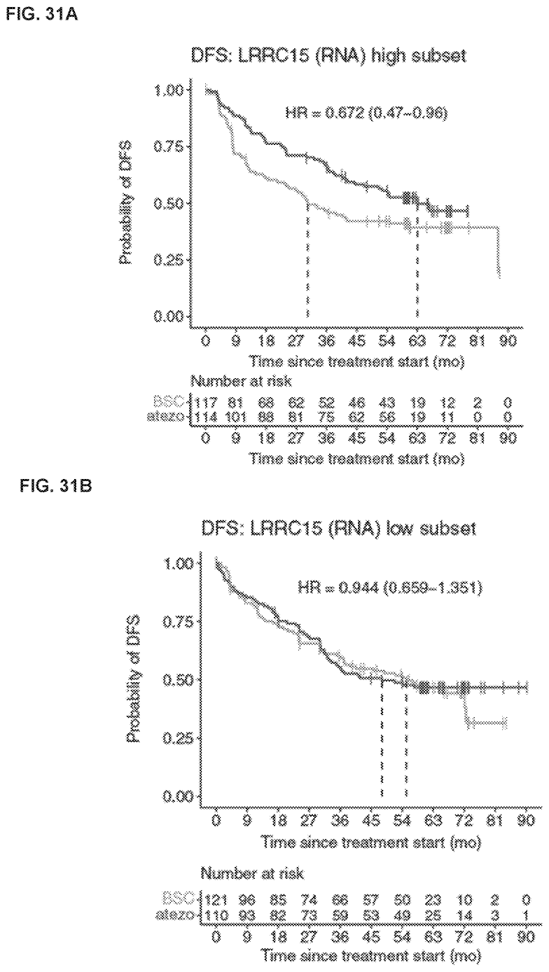

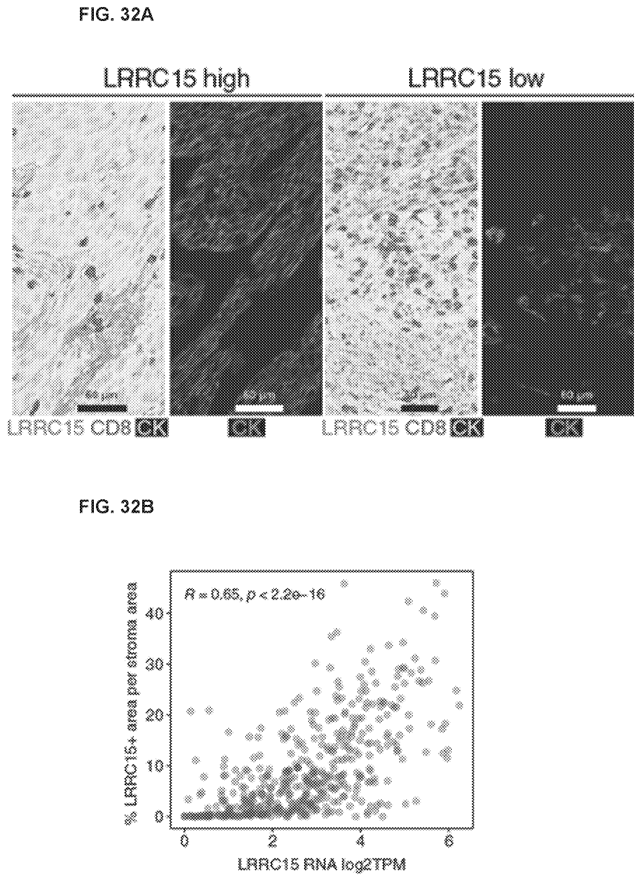

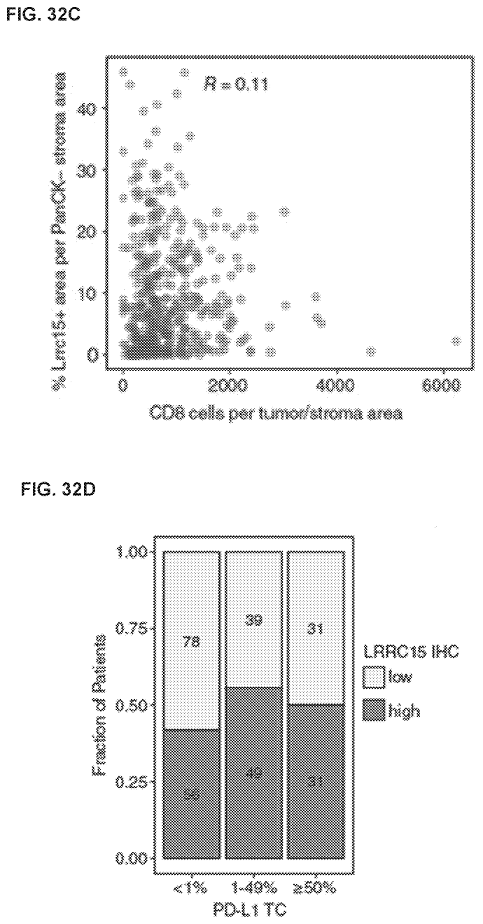

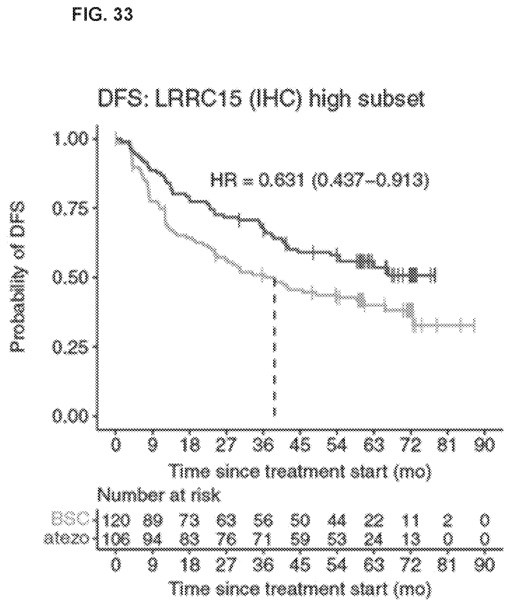

Further IHC analyses of LRRC15 as a single gene marker for TGFβ CAFs demonstrated the same outcome association as the TGFβ CAF gene signature, in which atezolizumab had longer DFS in the LRCC15-high expression subset compared to BSC (FIGS. 31A-31B). A three-plex hybrid immunohistochemistry and immunofluorescence (IHC/IF) assay for pan-cytokeratin (CK), CD8, and LRCC15 was developed and then used with 451 samples from the IMpower010 clinical study, in which bulk RNA-seq analyses were also performed (FIG. 32A). LRRC15 signal primarily localized to tumor stroma and highlighted cytoplasm and cytoplasmic processes of cells morphologically similar to fibroblasts with variable distance to CK-positive tumor cells. LRRC15 signal density was remarkably correlated with LRRC15 RNA expression in matched samples (R=0.65; FIG. 32B) but did not correlate with the amount of CD8 T cells (R=0.11) or PD-L1 status (FIGS. 32C-32D). Stratification of patients by LRRC15 expression as measured by IHC demonstrated that atezolizumab had longer DFS compared to BSC in individuals with high LRRC15 expression in tumor samples but not individuals that had low tumor LRRC15 expression (FIG. 33).

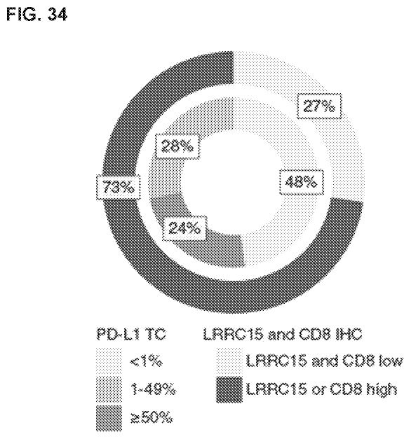

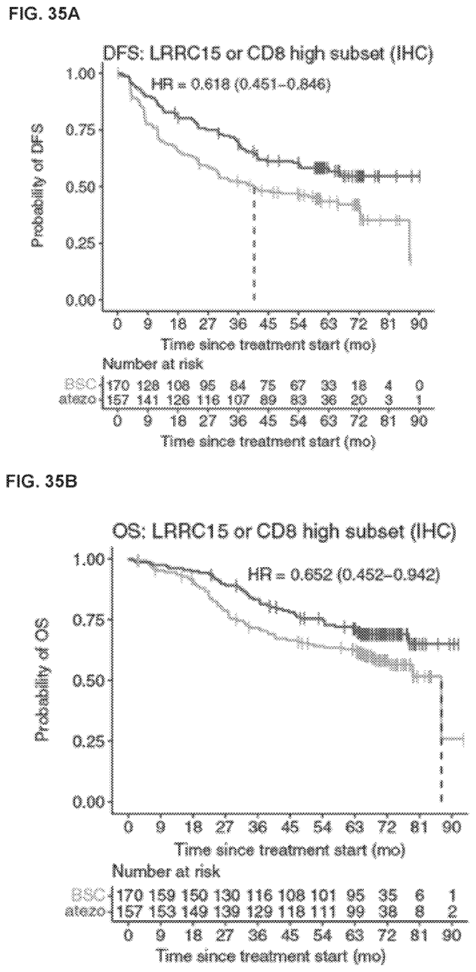

Because T-effector gene signatures and TGFβ CAF gene signatures were each independently associated with clinical benefit from treatment with atezolizumab, the IHC/IF assay was next implemented to assess both CD8 T cells and LRRC15 expression to identify patients that are most likely to benefit from atezolizumab treatment, as compared to BSC. Patients were stratified by the median of quantified CD8 T cells or LRRC15 expression, resulting in 72.5% of patients (327/449) classified as CD8-positive or LRRC15-positive, which was greater than the number of patients identified based on PD-L1 status, in which 52.1% of patients (234/449) had PD-L1>1% tumors and 24.1% of patients (108/449) had PD-L1≥50% tumors (FIG. 34). In this broader population of patients identified by CD8 T cells or high LRRC15 expression, treatment with atezolizumab resulted in longer DFS and OS as compared to BSC, which was similar to results stratified by patient tumor samples having high expression of the T-effector gene signature, PD-L1 expression≥1%, or high expression of the TGFβ CAF gene signature (FIGS. 35A-35B). These data therefore suggest that a single IHC/IF assay that quantifies both LRRC15 expression and CD8 T cells captured a majority of patients enrolled in the IMpower010 clinical study that benefit from adjuvant atezolizumab compared to BSC.

### Discussion

To understand molecular features that may independently associate with adjuvant atezolizumab, a curated list of signatures encompassing relevant cell types and states, as well as clinical features, were considered. A TGFβ CAF gene signature was identified as a potentially predictive marker of outcomes for atezolizumab compared with BSC independent of histology, PD-L1 status, and lymphocyte infiltration. Notably, for patients with high TGFβ CAF gene signature expression, a DFS improvement was seen with atezolizumab regardless of PD-L1 expression level. These data suggest that patients who are PD-L1 low or negative but TGFβ CAF-high may still derive a DFS benefit from atezolizumab. In summary, understanding TGFβ CAF gene signature status in combination with PD-L1 expression may be an important tool to identify the patient populations that are most likely to respond to atezolizumab.

To examine the validity of high TGFβ CAF as a negative prognostic factor for survival in other cancer settings, OS outcomes were evaluated in two independent data sets. Similar to the results in the early-stage NSCLC dataset, ctDNA+ patients with resected muscle-invasive urothelial carcinoma revealed that compared with low TGFβ CAF gene expression, high TGFβ CAF expression was associated with worse outcomes in the observation arm, with a similar trend in the atezolizumab arm. When examining OS by TGFβ subgroups in an advanced/metastatic NSCLC dataset, high TGFβ CAF expression was associated with worse outcomes compared to low TGFβ CAF expression in the atezolizumab arm. The pattern of recurrences and ctDNA status suggest that the TGFβ CAF-high primary tumors in the early-stage setting have disseminated prior to resection, which may therefore account for the poor prognosis in the control arms.

In conclusion, this analysis is the first to show an association between TGFβ CAF and clinical outcomes with an immune checkpoint inhibitor in patients with early-stage resected NSCLC. Data from the BSC arm showed that high expression of the TGFβ CAF gene signature may be a negative prognostic factor for DFS and OS. Further, these data suggest that patients with high expression of the TGFβ CAF gene signature may derive DFS and OS benefit from adjuvant atezolizumab compared with BSC after surgical resection, with DFS benefit observed regardless of histology or PD-L1 expression level. By extending this to development of a single IHC/IF assay, demonstrated the future feasibility for patient stratification or selection. As the use of immune checkpoint inhibitors becomes standard of care in eNSCLC, it is increasingly relevant to delineate potential biomarkers of clinical benefit for such inhibitors.

### Methods

The design of the IMpower010 clinical study and bulk RNA-sequencing was performed as described in Example 1.

### Immunohistochemistry/Immunofluorescence Assay

The assay was performed on 5 μm thick paraffin-embedded tissue sections using a Ventana Discovery Ultra staining platform (Roche Tissue Diagnostics (RTD), Tucson, AZ). The three markers were detected sequentially using the following primary antibodies at the indicated concentrations: LRRC15, clone EPR8188 (2), 2.5 μg/ml (AbCam Inc., Waltham, MA); CD8, clone SP16, 1:200, (AbCam Inc.); panCytokeratin (CK), clone AE1/AE3, 2 μg/ml (M351501-2, Agilent, Santa Clara, CA). Heat-induced epitope retrieval was performed using Cell Conditioning Solution 1 (Cat #950-500; RTD) for 64 minutes. Specifically bound primary antibodies were detected using Ventana Rabbit OmniMap (cat #760-4311; RTD) for LRRC15 and CD8 and Ventana Mouse OmniMap (cat #760-4310, RTD) for CK. The targets were visualized with Discovery Purple (cat #760-229; RTD) for LRRC15, ChromoMap DAB (cat #760-159; RTD) for CD8 and Discovery Cy5-TSA (cat #760-238, RTD) for CK.

Brightfield (RGB) and fluorescent (Cy5/CFP) images were acquired on the Nanozoomer S60. Pathologist annotations were made to define tumor-containing regions and analysis was restricted to tissue within these tumor regions-of-interest (ROIs). Tissue area, Tumor ROI area, PanCK+ area, and PanCK-stroma area were reported for each sample. Percent LRRC15 area was calculated for both PanCK+ and PanCK-stroma areas. CD8+ cell density (cells/mm2) was calculated for both PanCK+ and PanCK-stroma regions.

### Single Cell RNA-Sequencing

The single-cell RNA-seq data from Maynard et al. (Cell. 182 (5): 1232-1251, 2020) were downloaded as provided by the authors and analyzed in R. Only cells from the lung were retained. Raw counts were normalized to log (CP100K+1). Main cell types were identified based on clusters at a resolution of 0.1 and 30 principal components (PCs). Fibroblast and pericyte subsets were identified with a clustering resolution of 1 and 30 PCs. To identify CAF/CAP specific markers, differential expression analysis was performed comparing one CAF/CAP subset to all other CAF/CAPs only retaining genes fulfilling the following criteria for each subset: (i) The gene is expressed in >30% of cells in the subset under consideration, but in less than 25% of cells in the other subset; (ii) The average log 2FC is >0.5; and (iii) the adjusted p-value is <1e-40. Genes that might be expressed by non-CAF/CAPs were excluded by removing genes that were expressed by a cumulative cell fraction>0.35 summing over the fraction of positive cells for T cells (proliferating and non-proliferating), TAMs, Endothelial cells, Plasma cells, B cells, Mast cells, Neuronal cells, Low Quality cells, Epithelial cells and Pericytes for CAFs or Fibroblasts for CAPs, respectively.

### Statistics

DFS and OS data were based on the clinical cutoff date of Jan. 26, 2024. Median DFS and OS were estimated using Kaplan-Meier curves. Unstratified hazard ratios (HR) were estimated by Cox-proportional hazard models.

### OTHER EMBODIMENTS

Although the foregoing invention has been described in some detail by way of illustration and example for purposes of clarity of understanding, the descriptions and examples should not be construed as limiting the scope of the invention.

What is claimed is:

1. A method for treating a non-small cell lung cancer (NSCLC) in an individual, the method comprising:
(a) determining the expression level of a transforming growth factor-beta (TGFβ) cancer-associated fibroblast (CAF) gene signature in a tumor tissue sample obtained from the individual, wherein an expression level of the TGFβ CAF gene signature has been determined to be at or above a reference TGFβ CAF gene signature expression level, thereby indicating that the individual is likely to have an increased clinical benefit from an adjuvant treatment comprising an anti-PD-L1 antibody compared to treatment with best supportive care (BSC); and
(b) administering an effective amount of the anti-PD-L1 antibody to the individual,
wherein the anti-PD-L1 antibody comprises the following hypervariable regions (HVRs):
(i) an HVR-H1 sequence of GFTFSDSWIH (SEQ ID NO: 3);
(ii) an HVR-H2 sequence of AWISPYGGSTYYADSVKG (SEQ ID NO: 4);
(iii) an HVR-H3 sequence of RHWPGGFDY (SEQ ID NO: 5);
(iv) an HVR-L1 sequence of RASQDVSTAVA (SEQ ID NO: 6);
(v) an HVR-L2 sequence of SASFLYS (SEQ ID NO: 7); and
(vi) an HVR-L3 sequence of QQYLYHPAT (SEQ ID NO: 8).

2. A method of treating an individual having an NSCLC, the method comprising administering to the individual an effective amount of an anti-PD-L1 antibody, wherein a tumor tissue sample from the individual has been determined to have an expression level of a TGFβ CAF gene signature that is at or above a reference TGFβ CAF gene signature expression level, thereby indicating that the individual is likely to have an increased clinical benefit from an adjuvant treatment comprising the anti-PD-L1 antibody compared to treatment with BSC, and
wherein the anti-PD-L1 antibody comprises the following HVRs:
(i) an HVR-H1 sequence of GFTFSDSWIH (SEQ ID NO: 3);
(ii) an HVR-H2 sequence of AWISPYGGSTYYADSVKG (SEQ ID NO: 4);
(iii) an HVR-H3 sequence of RHWPGGFDY (SEQ ID NO: 5);
(iv) an HVR-L1 sequence of RASQDVSTAVA (SEQ ID NO: 6);
(v) an HVR-L2 sequence of SASFLYS (SEQ ID NO: 7); and
(vi) an HVR-L3 sequence of QQYLYHPAT (SEQ ID NO: 8).

3. A method of identifying an individual having an NSCLC who is likely to have an increased clinical benefit from an adjuvant treatment comprising an anti-PD-L1 antibody compared to treatment with BSC, the method comprising determining the expression level of a TGFβ CAF gene signature in a tumor tissue sample obtained from the individual, wherein an expression level of the TGFβ CAF gene signature has been determined to be at or above a reference TGFβ CAF gene signature expression level, thereby indicating that the individual is likely to have an increased clinical benefit from the adjuvant treatment comprising the anti-PD-L1 antibody compared to treatment with BSC,
wherein the anti-PD-L1 antibody comprises the following HVRs:
(i) an HVR-H1 sequence of GFTFSDSWIH (SEQ ID NO: 3);
(ii) an HVR-H2 sequence of AWISPYGGSTYYADSVKG (SEQ ID NO: 4);
(iii) an HVR-H3 sequence of RHWPGGFDY (SEQ ID NO: 5);
(iv) an HVR-L1 sequence of RASQDVSTAVA (SEQ ID NO: 6);
(v) an HVR-L2 sequence of SASFLYS (SEQ ID NO: 7); and
(vi) an HVR-L3 sequence of QQYLYHPAT (SEQ ID NO: 8).

4. A method for selecting a therapy for an individual having an NSCLC, the method comprising determining the expression level of a TGFβ CAF gene signature in a tumor tissue sample obtained from the individual, wherein an expression level of the TGFβ CAF gene signature has been determined to be at or above a reference TGFβ CAF gene signature expression level, thereby indicating that the individual is likely to have an increased clinical benefit from an adjuvant treatment comprising an anti-PD-L1 antibody compared to treatment with BSC,
wherein the anti-PD-L1 antibody comprises the following HVRs:
(i) an HVR-H1 sequence of GFTFSDSWIH (SEQ ID NO: 3);
(ii) an HVR-H2 sequence of AWISPYGGSTYYADSVKG (SEQ ID NO: 4);
(iii) an HVR-H3 sequence of RHWPGGFDY (SEQ ID NO: 5);
(iv) an HVR-L1 sequence of RASQDVSTAVA (SEQ ID NO: 6);
(v) an HVR-L2 sequence of SASFLYS (SEQ ID NO: 7); and
(vi) an HVR-L3 sequence of QQYLYHPAT (SEQ ID NO: 8).

5. The method of claim 3 or 4, wherein the method further comprises administering to the individual an effective amount of the anti-PD-L1 antibody.

6. A kit for identifying an individual having an NSCLC who is likely to have an increased clinical benefit from an adjuvant treatment comprising an anti-PD-L1 antibody compared to treatment with BSC, the kit comprising reagents for determining the expression level of a TGFβ CAF gene signature in a tumor tissue sample obtained from the individual, wherein an expression level of a TGFβ CAF gene signature that is at or above a reference TGFβ CAF gene signature expression level identifies that the individual as one who is likely to have an increased clinical benefit from the adjuvant treatment comprising the anti-PD-L1 antibody compared to treatment with BSC,
wherein the anti-PD-L1 antibody comprises the following HVRs:
(i) an HVR-H1 sequence of GFTFSDSWIH (SEQ ID NO: 3);
(ii) an HVR-H2 sequence of AWISPYGGSTYYADSVKG (SEQ ID NO: 4);
(iii) an HVR-H3 sequence of RHWPGGFDY (SEQ ID NO: 5);
(iv) an HVR-L1 sequence of RASQDVSTAVA (SEQ ID NO: 6);
(v) an HVR-L2 sequence of SASFLYS (SEQ ID NO: 7); and
(vi) an HVR-L3 sequence of QQYLYHPAT (SEQ ID NO: 8).

7. An assay for identifying an individual having an NSCLC who is a candidate for an adjuvant treatment comprising an anti-PD-L1 antibody, the assay comprising determining the expression level of a TGFβ CAF gene signature in a tumor tissue sample obtained from the individual, wherein an expression level of the TGFβ CAF gene signature has been determined to be at or above a reference TGFβ CAF gene signature expression level, thereby indicating that the individual is likely to have an increased clinical benefit from the adjuvant treatment comprising the anti-PD-L1 antibody compared to treatment with BSC,
wherein the anti-PD-L1 antibody comprises the following HVRs:
(i) an HVR-H1 sequence of GFTFSDSWIH (SEQ ID NO: 3);
(ii) an HVR-H2 sequence of AWISPYGGSTYYADSVKG (SEQ ID NO: 4);
(iii) an HVR-H3 sequence of RHWPGGFDY (SEQ ID NO: 5);
(iv) an HVR-L1 sequence of RASQDVSTAVA (SEQ ID NO: 6);
(v) an HVR-L2 sequence of SASFLYS (SEQ ID NO: 7); and
(vi) an HVR-L3 sequence of QQYLYHPAT (SEQ ID NO: 8).

8. A method for treating an NSCLC in an individual, the method comprising:
(a) determining the expression level of LRRC15 in a tumor tissue sample obtained from the individual, wherein an expression level of LRRC15 has been determined to be at or above a reference LRRC15 expression level, thereby indicating that the individual is likely to have an increased clinical benefit from an adjuvant treatment comprising an anti-PD-L1 antibody compared to treatment with BSC; and
(b) administering an effective amount of the anti-PD-L1 antibody to the individual,
wherein the anti-PD-L1 antibody comprises the following hypervariable regions (HVRs):
(i) an HVR-H1 sequence of GFTFSDSWIH (SEQ ID NO: 3);
(ii) an HVR-H2 sequence of AWISPYGGSTYYADSVKG (SEQ ID NO: 4);
(iii) an HVR-H3 sequence of RHWPGGFDY (SEQ ID NO: 5);
(iv) an HVR-L1 sequence of RASQDVSTAVA (SEQ ID NO: 6);
(v) an HVR-L2 sequence of SASFLYS (SEQ ID NO: 7); and
(vi) an HVR-L3 sequence of QQYLYHPAT (SEQ ID NO: 8).

9. A method of treating an individual having an NSCLC, the method comprising administering to the individual an effective amount of an anti-PD-L1 antibody, wherein a tumor tissue sample from the individual has been determined to have an expression level of LRRC15 that is at or above a reference LRRC15 expression level, thereby indicating that the individual is likely to have an increased clinical benefit from an adjuvant treatment comprising the anti-PD-L1 antibody compared to treatment with BSC, and
wherein the anti-PD-L1 antibody comprises the following HVRs:
(i) an HVR-H1 sequence of GFTFSDSWIH (SEQ ID NO: 3);
(ii) an HVR-H2 sequence of AWISPYGGSTYYADSVKG (SEQ ID NO: 4);
(iii) an HVR-H3 sequence of RHWPGGFDY (SEQ ID NO: 5);
(iv) an HVR-L1 sequence of RASQDVSTAVA (SEQ ID NO: 6);
(v) an HVR-L2 sequence of SASFLYS (SEQ ID NO: 7); and
(vi) an HVR-L3 sequence of QQYLYHPAT (SEQ ID NO: 8).

10. A method of identifying an individual having an NSCLC who is likely to have an increased clinical benefit from an adjuvant treatment comprising an anti-PD-L1 antibody compared to treatment with BSC, the method comprising determining the expression level of LRRC15 in a tumor tissue sample obtained from the individual, wherein an expression level of LRRC15 has been determined to be at or above a reference LRRC15 expression level, thereby indicating that the individual is likely to have an increased clinical benefit from the adjuvant treatment comprising the anti-PD-L1 antibody compared to treatment with BSC,
wherein the anti-PD-L1 antibody comprises the following HVRs:
(i) an HVR-H1 sequence of GFTFSDSWIH (SEQ ID NO: 3);
(ii) an HVR-H2 sequence of AWISPYGGSTYYADSVKG (SEQ ID NO: 4);
(iii) an HVR-H3 sequence of RHWPGGFDY (SEQ ID NO: 5);
(iv) an HVR-L1 sequence of RASQDVSTAVA (SEQ ID NO: 6);
(v) an HVR-L2 sequence of SASFLYS (SEQ ID NO: 7); and
(vi) an HVR-L3 sequence of QQYLYHPAT (SEQ ID NO: 8).

11. A method for selecting a therapy for an individual having an NSCLC, the method comprising determining the expression level of LRRC15 in a tumor tissue sample obtained from the individual, wherein an expression level of LRRC15 has been determined to be at or above a reference LRRC15 expression level, thereby indicating that the individual is likely to have an increased clinical benefit from an adjuvant treatment comprising an anti-PD-L1 antibody compared to treatment with BSC,
wherein the anti-PD-L1 antibody comprises the following HVRs:
(i) an HVR-H1 sequence of GFTFSDSWIH (SEQ ID NO: 3);
(ii) an HVR-H2 sequence of AWISPYGGSTYYADSVKG (SEQ ID NO: 4);
(iii) an HVR-H3 sequence of RHWPGGFDY (SEQ ID NO: 5);
(iv) an HVR-L1 sequence of RASQDVSTAVA (SEQ ID NO: 6);
(v) an HVR-L2 sequence of SASFLYS (SEQ ID NO: 7); and
(vi) an HVR-L3 sequence of QQYLYHPAT (SEQ ID NO: 8).

12. The method of claim 10 or 11, wherein the method further comprises administering to the individual an effective amount of the anti-PD-L1 antibody.

13. A kit for identifying an individual having an NSCLC who is likely to have an increased clinical benefit from an adjuvant treatment comprising an anti-PD-L1 antibody compared to treatment with BSC, the kit comprising reagents for determining the expression level of LRRC15 in a tumor tissue sample obtained from the individual, wherein an expression level of LRRC15 that is at or above a reference LRRC15 expression level identifies that the individual as one who is likely to have an increased clinical benefit from the adjuvant treatment comprising the anti-PD-L1 antibody compared to treatment with BSC,
wherein the anti-PD-L1 antibody comprises the following HVRs:
(i) an HVR-H1 sequence of GFTFSDSWIH (SEQ ID NO: 3);
(ii) an HVR-H2 sequence of AWISPYGGSTYYADSVKG (SEQ ID NO: 4);
(iii) an HVR-H3 sequence of RHWPGGFDY (SEQ ID NO: 5);
(iv) an HVR-L1 sequence of RASQDVSTAVA (SEQ ID NO: 6);
(v) an HVR-L2 sequence of SASFLYS (SEQ ID NO: 7); and
(vi) an HVR-L3 sequence of QQYLYHPAT (SEQ ID NO: 8).

14. An assay for identifying an individual having an NSCLC who is a candidate for an adjuvant treatment comprising an anti-PD-L1 antibody, the assay comprising determining the expression level of LRRC15 in a tumor tissue sample obtained from the individual, wherein an expression level of LRRC15 has been determined to be at or above a reference LRRC15 expression level, thereby indicating that the individual is likely to have an increased clinical benefit from the adjuvant treatment comprising the anti-PD-L1 antibody compared to treatment with BSC,
wherein the anti-PD-L1 antibody comprises the following HVRs:
(i) an HVR-H1 sequence of GFTFSDSWIH (SEQ ID NO: 3);
(ii) an HVR-H2 sequence of AWISPYGGSTYYADSVKG (SEQ ID NO: 4);
(iii) an HVR-H3 sequence of RHWPGGFDY (SEQ ID NO: 5);
(iv) an HVR-L1 sequence of RASQDVSTAVA (SEQ ID NO: 6);
(v) an HVR-L2 sequence of SASFLYS (SEQ ID NO: 7); and
(vi) an HVR-L3 sequence of QQYLYHPAT (SEQ ID NO: 8).

15. The method, kit, or assay of any one of claims 1-14, wherein the anti-PD-L1 antibody comprises:
(a) a VH comprising the amino acid sequence: EVOLVESGGGLVQPGGSLRLSCAASGFTFSDSWIHWVRQAPGKGLEWVAWISPYGGSTYYADSVKGRF TISADTSKNTAYLQMNSLRAEDTAVYYCARRHWPGGFDYWGQGTLVTVSS (SEQ ID NO: 9), and
(b) a VL comprising the amino acid sequence:
(SEQ ID NO: 10)
DIQMTQSPSSLSASVGDRVTITCRASQDVSTAVAWYQQKPGKAPKLLIYSA
SFLYSGVPSRFSGSGSGTDFTLTISSLOPEDFATYYCQQYLYHPATFGQGT
KVEIKR.

16. The method, kit, or assay of any one of claims 1-15, wherein the anti-PD-L1 antibody comprises:
(a) a heavy chain comprising amino acid sequence: EVOLVESGGGLVQPGGSLRLSCAASGFTFSDSWIHWVRQAPGKGLEWVAWISPYGGSTYYADSVKGRF TISADTSKNTAYLQMNSLRAEDTAVYYCARRHWPGGFDYWGQGTLVTVSSASTKGPSVFPLAPSSKSTS GGTAALGCLVKDYFPEPVTVSWNSGALTSGVHTFPAVLQSSGLYSLSSVVTVPSSSLGTQTYICNVNHKP SNTKVDKKVEPKSCDKTHTCPPCPAPELLGGPSVFLFPPKPKDTLMISRTPEVTCVVVDVSHEDPEVKFN WYVDGVEVHNAKTKPREEQYASTYRVVSVLTVLHQDWLNGKEYKCKVSNKALPAPIEKTISKAKGQPRE PQVYTLPPSREEMTKNQVSLTCLVKGFYPSDIAVEWESNGQPENNYKTTPPVLDSDGSFFLYSKLTVDKS RWQQGNVFSCSVMHEALHNHYTQKSLSLSPG (SEQ ID NO: 1), and
(b) a light chain comprising the amino acid sequence:
(SEQ ID NO: 2)
DIQMTQSPSSLSASVGDRVTITCRASQDVSTAVAWYQQKPGKAPKLLIYSA
SFLYSGVPSRFSGSGSGTDFTLTISSLQPEDFATYYCQQYLYHPATFGQGT
KVEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSP
VTKSFNRGEC.

17. The method, kit, or assay of any one of claims 1-16, wherein the anti-PD-L1 antibody is atezolizumab.

18. The method, kit, or assay of any one of claims 1-17, wherein the adjuvant treatment comprises intravenous or subcutaneous administration of the anti-PD-L1 antibody to the individual.

19. The method, kit, or assay of any one of claims 1-18, wherein the adjuvant treatment comprises administration of the anti-PD-L1 antibody as a monotherapy.

20. The method, kit, or assay of any one of claims 1-19, wherein the adjuvant treatment comprises intravenous administration of the anti-PD-L1 antibody to the individual at a dose of 1200 mg every 3 weeks.

21. The method, kit, or assay of any one of claims 1-19, wherein the adjuvant treatment comprises subcutaneous administration of the anti-PD-L1 antibody to the individual at a dose of 1875 mg every 3 weeks.

22. The method, kit, or assay of claim 21, wherein the adjuvant treatment comprises administration of the anti-PD-L1 antibody for up to 1 year.

23. The method, kit, or assay of any one of claims 1-22, wherein the individual has previously received a platinum-based adjuvant therapy.

24. The method, kit, or assay of claim 23, wherein the platinum-based adjuvant therapy comprises cisplatin.

25. The method, kit, or assay of claim 23 or 24, wherein the platinum-based adjuvant therapy further comprises one or more additional chemotherapeutic agents.

26. The method, kit, or assay of claim 25, wherein the one or more additional chemotherapeutic agents comprises a vinca alkaloid, a taxane, an anti-metabolite, or a combination thereof.

27. The method, kit, or assay of claim 26, wherein the vinca alkaloid is vinorelbine.

28. The method, kit, or assay of claim 26, wherein the taxane is docetaxel.

29. The method, kit, or assay of claim 26, wherein the anti-metabolite is gemcitabine or pemetrexed.

30. The method, kit, or assay of claim 29, wherein the anti-metabolite is gemcitabine.

31. The method, kit, or assay of claim 29, wherein the anti-metabolite is pemetrexed.

32. The method, kit, or assay of any one of claims 23-31, wherein the platinum-based adjuvant therapy comprises up to four cycles.

33. The method, kit, or assay of any one of claims 23-32, wherein the platinum-based adjuvant therapy comprises up to four 21-day cycles.

34. The method, kit, or assay of claim 33, wherein the platinum-based adjuvant therapy comprises cisplatin, wherein cisplatin was administered at a dose of about 75 mg/m² intravenously on Day 1 of each 21-day cycle.

35. The method, kit, or assay of claim 33, wherein the platinum-based adjuvant therapy comprises cisplatin and vinorelbine, wherein cisplatin was administered at a dose of about 75 mg/m² intravenously on Day 1 of each 21 day-cycle, and vinorelbine was administered at a dose of about 30 mg/m² by intravenous push on Days 1 and 8 of each 21-day cycle.

36. The method, kit, or assay of claim 33, wherein the platinum-based adjuvant therapy comprises cisplatin and docetaxel, wherein cisplatin was administered at a dose of about 75 mg/m² intravenously on Day 1 of each 21 day-cycle, and docetaxel was administered at a dose of about 75 mg/m² intravenously on Day 1 of each 21-day cycle.

37. The method, kit, or assay of claim 33, wherein the platinum-based adjuvant therapy comprises cisplatin and gemcitabine, wherein cisplatin was administered at a dose of about 75 mg/m² intravenously on Day 1 of each 21 day-cycle, and gemcitabine was administered at a dose of about 1250 mg/m² intravenously on Days 1 and 8 of each 21-day cycle.

38. The method, kit, or assay of claim 33, wherein the platinum-based adjuvant therapy comprises cisplatin and pemetrexed, wherein cisplatin was administered at a dose of about 75 mg/m² intravenously on Day 1 of each 21 day-cycle, and pemetrexed was administered at a dose of about 500 mg/m² intravenously on Day 1 of each 21-day cycle.

39. The method, kit, or assay of any one of claims 1-38, wherein the NSCLC is stage IB, IIA, IIB, or IIIA.

40. The method, kit, or assay of claim 39, wherein the NSCLC staging is per the Union Internationale Contre le Cancer/American Joint Committee on Cancer (UICC/AJCC) staging system, 7^{th} edition.

41. The method, kit, or assay of any one of claims 1-40, wherein the NSCLC is not an EGFR/ALK+ NSCLC.

42. The method, kit, or assay of any one of claims 1-41, wherein the NSCLC is squamous or non-squamous NSCLC.

43. The method, kit, or assay of claim 42, wherein the NSCLC is squamous NSCLC.

44. The method, kit, or assay of claim 42, wherein the NSCLC is non-squamous NSCLC.

45. The method, kit, or assay of any one of claims 23-44, wherein the individual had a complete resection of the NSCLC prior to the platinum-based adjuvant therapy.

46. The method, kit, or assay of claim 45, wherein the complete resection comprises lobectomy, sleeve lobectomy, bilobectomy, or pneumonectomy.

47. The method, kit, or assay of claim 45 or 46, wherein the individual had complete resection of the NSCLC about 4 to about 12 weeks prior to the platinum-based adjuvant therapy.

48. The method, kit, or assay of any one of claims 23-47, wherein the individual has not had treatment with systemic chemotherapy prior to the platinum-based adjuvant therapy.

49. The method, kit, or assay of any one of claims 1-48, wherein the increased clinical benefit is an increase in disease-free survival (DFS).

50. The method, kit, or assay of any one of claims 1-49, wherein the increased clinical benefit is an increase in overall survival (OS).

51. The method, kit, or assay of any one of claims 1-50, wherein the tumor tissue sample is obtained from the individual prior to administration of the anti-PD-L1 antibody.

52. The method, kit, or assay of any one of claims 1-51, wherein the tumor tissue sample is obtained from the individual prior to administration of a platinum-based adjuvant therapy.

53. The method, kit, or assay of any one of claims 1-7 and 15-52, wherein the reference TGFβ CAF gene signature expression level is the median expression level of the TGFβ CAF gene signature in a reference population.

54. The method, kit, or assay of any one of claims 1-7 and 15-53, wherein the expression level of the TGFβ CAF gene signature is the median of the expression levels of a set of genes comprising the TGFβ CAF gene signature.

55. The method, kit, or assay of claim 54, wherein the set of genes comprising the TGFβ CAF gene signature comprises the following genes: LRRC15, FAP, COL11A1, COL10A1, FNDC1, GREM1, ITGA11, WISP1, PLPP4, PODNL1, TWIST1, COL8A2, SUGCT, NOX4, ZNF469, TENM3, P4HA3, ADAMTS12, and CPZ.

56. The method, kit, or assay of claim 54 or 55, wherein the set of genes comprising the TGFβ CAF gene signature consists of LRRC15, FAP, COL11A1, COL10A1, FNDC1, GREM1, ITGA11, WISP1, PLPP4, PODNL1, TWIST1, COL8A2, SUGCT, NOX4, ZNF469, TENM3, P4HA3, ADAMTS12, and CPZ.

57. The method, kit, or assay of any one of claims 8-52, wherein the reference expression level of LRRC15 is the median expression level of LRRC15 in a reference population.

58. The method, kit, or assay of any one of claims 53-57, wherein the reference population comprises a set of individuals having the NSCLC.

59. The method, kit, or assay of any one of claims 1-58, wherein the expression level is an RNA expression level.

60. The method, kit, or assay of claim 59, wherein the RNA expression level is determined using RNA sequencing.

61. The method, kit, or assay of claim 60, wherein the RNA sequencing is performed using an Illumina sequencing-by-synthesis platform.

62. The method, kit, or assay of claim 61, wherein the Illumina sequencing-by-synthesis platform is a NOVASEQ® 6000.

63. The method, kit, or assay of any one of claims 60-62, wherein the RNA sequencing is performed on a library generated using a RIBO-ZERO® Magnetic Gold Kit.

64. The method, kit, or assay of any one of claims 8-52, 57, and 58, wherein the expression level of LRRC15 is a protein expression level.

65. The method, kit, or assay of claim 64, wherein the protein expression level is determined by immunohistochemistry.

66. The method, kit, or assay of claim 65, wherein the immunohistochemistry is performed using a Ventana staining platform.

67. A pharmaceutical composition comprising an anti-PD-L1 antibody for use in an adjuvant treatment of an individual having an NSCLC, wherein the adjuvant treatment comprises administration of an effective amount of the anti-PD-L1 antibody, wherein a tumor tissue sample obtained from the individual has been determined to have an expression level of an TGFβ CAF gene signature that is at or above a reference TGFβ CAF gene signature expression level, thereby indicating that the individual is likely to have an increased clinical benefit from treatment with the anti-PD-L1 antibody compared to treatment with BSC, and
wherein the anti-PD-L1 antibody comprises the following HVRs:
(i) an HVR-H1 sequence of GFTFSDSWIH (SEQ ID NO: 3);
(ii) an HVR-H2 sequence of AWISPYGGSTYYADSVKG (SEQ ID NO: 4);
(iii) an HVR-H3 sequence of RHWPGGFDY (SEQ ID NO: 5);
(iv) an HVR-L1 sequence of RASQDVSTAVA (SEQ ID NO: 6);
(v) an HVR-L2 sequence of SASFLYS (SEQ ID NO: 7); and
(vi) an HVR-L3 sequence of QQYLYHPAT (SEQ ID NO: 8).

68. Use of an anti-PD-L1 antibody in the manufacture of a medicament for adjuvant treatment of an individual having an NSCLC, wherein the adjuvant treatment comprises administration of an effective amount of the anti-PD-L1 antibody, wherein a tumor tissue sample obtained from the individual has been determined to have an expression level of an TGFβ CAF gene signature that is at or above a reference TGFβ CAF gene signature expression level, thereby indicating that the individual is likely to have an increased clinical benefit from treatment with the anti-PD-L1 antibody compared to treatment with BSC, and
wherein the anti-PD-L1 antibody comprises the following HVRs:
(i) an HVR-H1 sequence of GFTFSDSWIH (SEQ ID NO: 3);
(ii) an HVR-H2 sequence of AWISPYGGSTYYADSVKG (SEQ ID NO: 4);
(iii) an HVR-H3 sequence of RHWPGGFDY (SEQ ID NO: 5);
(iv) an HVR-L1 sequence of RASQDVSTAVA (SEQ ID NO: 6);
(v) an HVR-L2 sequence of SASFLYS (SEQ ID NO: 7); and
(vi) an HVR-L3 sequence of QQYLYHPAT (SEQ ID NO: 8).

69. An article of manufacture comprising an anti-PD-L1 antibody and instructions to administer the anti-PD-L1 antibody for adjuvant treatment of an individual having an NSCLC, wherein the adjuvant treatment comprises administration of an effective amount of the anti-PD-L1 antibody, wherein a tumor tissue sample obtained from the individual has been determined to have an expression level of an TGFβ CAF gene signature that is at or above a reference TGFβ CAF gene signature expression level, thereby indicating that the individual is likely to have an increased clinical benefit from the adjuvant treatment comprising the anti-PD-L1 antibody compared to treatment with BSC, and
wherein the anti-PD-L1 antibody comprises the following HVRs:
(i) an HVR-H1 sequence of GFTFSDSWIH (SEQ ID NO: 3);
(ii) an HVR-H2 sequence of AWISPYGGSTYYADSVKG (SEQ ID NO: 4);
(iii) an HVR-H3 sequence of RHWPGGFDY (SEQ ID NO: 5);
(iv) an HVR-L1 sequence of RASQDVSTAVA (SEQ ID NO: 6);
(v) an HVR-L2 sequence of SASFLYS (SEQ ID NO: 7); and
(vi) an HVR-L3 sequence of QQYLYHPAT (SEQ ID NO: 8).

70. A method of treating an individual comprising administering to the individual an adjuvant treatment comprising an effective amount of atezolizumab following complete resection and no progression after platinum-based chemotherapy for an individual with stage IB to IIIA NSCLC, wherein a tumor tissue sample obtained from the individual has been determined to have an expression level of an TGFβ CAF gene signature that is at or above a reference TGFβ CAF gene signature expression level, thereby indicating that the individual is likely to have an increased DFS and/or OS after the adjuvant treatment comprising the atezolizumab compared to treatment with BSC,
wherein the expression level of the TGFβ CAF gene signature is the median RNA expression level of a set of genes comprising LRRC15, FAP, COL11A1, COL10A1, FNDC1, GREM1, ITGA11, WISP1, PLPP4, PODNL1, TWIST1, COL8A2, SUGCT, NOX4, ZNF469, TENM3, P4HA3, ADAMTS12, and CPZ, and
wherein the reference TGFβ CAF gene signature expression level is the median expression level of the TGFβ CAF gene signature in a reference population of individuals having stage IB to IIIA NSCLC.

71. A method of treating an individual comprising administering to the individual an adjuvant treatment comprising an effective amount of atezolizumab following complete resection and no progression after platinum-based chemotherapy for an individual with stage IB to IIIA NSCLC, wherein the tumor tissue sample from the individual has been determined to have an RNA expression level of LRRC15 that is at or above a median RNA expression level of LRRC15 in a reference population of individuals having stage IB to IIIA NSCLC, thereby indicating that the individual is likely to have an increased DFS and/or OS after adjuvant treatment comprising the atezolizumab compared to treatment with BSC.


SEQUENCE LISTING (10 sequences)

SEQ ID NO: 1 - protein, 447 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 214 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 10 aa, synthetic construct
GFTFSDSWIH

SEQ ID NO: 4 - protein, 18 aa, synthetic construct
AWISPYGGSTYYADSVKG

SEQ ID NO: 5 - protein, 9 aa, synthetic construct
RHWPGGFDY

SEQ ID NO: 6 - protein, 11 aa, synthetic construct
RASQDVSTAVA

SEQ ID NO: 7 - protein, 7 aa, synthetic construct
SASFLYS

SEQ ID NO: 8 - protein, 9 aa, synthetic construct
QQYLYHPAT

SEQ ID NO: 9 - protein, 118 aa, synthetic construct [sequence omitted]

SEQ ID NO: 10 - protein, 108 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR